(19) United States
(12) Patent Application Publication
(10) Pub. No.: US 2022/0096574 A1
(43) Pub. Date: Mar. 31, 2022

(54) METHODS AND COMPOSITIONS FOR TREATING AND PREVENTING CENTRAL NERVOUS SYSTEM DISORDERS AND OTHER CONDITIONS CAUSED BY GUT MICROBIAL DYSBIOSIS

(71) Applicant: HOLOBIOME, INC., Cambridge, MA (US)

(72) Inventors: STEPHEN SKOLNICK, SOMERVILLE, MA (US); PHILIP STRANDWITZ, MEDFORD, MA (US)

(73) Assignee: HOLOBIOME, INC., Cambridge, MA (US)

(21) Appl. No.: 17/426,178

(22) PCT Filed: Jan. 29, 2020
(86) PCT No.: PCT/US20/15728

§ 371 Date: Jul. 28, 2021

Related U.S. Application Data
(60) Provisional application No. 62/798,296, filed on Jan. 29, 2019.

(51) Int. Cl. A61K 35/747; A23L 33/135; A23L 33/14; A61K 31/49; A61K 31/714; A61K 35/742

(52) U.S. Cl. CPC A61K 35/747; A23L 33/135; A61K 35/742; A61K 31/49; A61K 31/714; A23L 33/14

(57) ABSTRACT

The technology described herein is directed to compositions and methods for treating CNS diseases or disorders associated with microbiome dysbiosis. In one aspect, described herein are compositions and methods for treating CNS diseases or disorders associated with a microbiome deficient in queuine biosynthesis. In another aspect, described herein are compositions and methods for treating CNS diseases or disorders associated with a microbiome deficient in endozepine biosynthesis. In another aspect, described herein are compositions and methods for treating CNS diseases or disorders associated with a microbiome deficient in heavy metal sequestration.

### CROSS-REFERENCE TO RELATED APPLICATIONS

This application claims benefit under 35 U.S.C. § 119(e) of U.S. Provisional Application No. 62/798,296 filed Jan. 29, 2019, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Jan. 29, 2020, is 251,702,365 total bytes in size and is submitted via EFS-Web in three pieces on Jan. 29, 2020: the first, submitted herewith, is named 083103-096850WOPT_SL_1.txt and is 77,747,143 bytes in size; the second, submitted subsequently associated with the PCT application number, is named 083103-096850WOPT_SL_2.txt and is 85,369,051 bytes in size; and the third, submitted subsequently associated with the PCT application number, is named 083103-096850WOPT_SL_3.txt and is 88,586,171 bytes in size.

### TECHNICAL FIELD

The technology described herein relates to prevention and treatment of dysbiosis caused by deficiencies or adverse changes in mammalian gut microbiology. More specifically, the technology described herein relates to methods and compositions for diagnosing and treating adverse health conditions in mammals caused by changes in gut microbial species composition, health, biology, activity and/or environment.

### BACKGROUND

Humans and other mammals depend on enteric microbes for their health and survival. These gut microbes initially colonize the mammalian gut from the environment, often directly or indirectly transmitted from other mammals of the same or different species. Essential enteric microbes can even be transmitted directly from parent to child. For example, recent studies reveal that mammal's milk contains live microorganisms from the GI tract of the mother selected by dendritic cells of the immune system. This vertical transmittal of critical microbes from parent to child to colonize the offspring's gut affords the offspring access to critical microbial processes and products. In evolutionary terms this constitutes a form of genetic “inheritance”—involving transfer of an adaptive complement of microbes and an associated library of microbial “ex-genes”, reliably and reproducibly from one generation to the next.

To maintain health, survival and adaptive fitness in evolution, mammals require certain microbes (and their ex-genes) to accompany individuals throughout life, to be practically re-acquirable if lost, and in the case of vertically transmitted species to be passed on with strict fidelity to subsequent generations. The extraordinary and complex capacity for vertical transmission of gut microbial species in breast milk indicates that symbioses between mammals and enteric microbes is far more critical and fundamental than previously suspected. Indeed, certain symbiotic associations between mammals and gut microbes have become so codependent, that mammals have lost the capacity to synthesize some essential products encoded by microbial genes. These critical “ex-genes”, and the microbial symbionts that carry them as quasi-heritable vectors, are linked in the mammal's life, reproduction and evolution by high fidelity transmittal, acquisition and colonization of microbes within the host gut and depend on reliable maintenance of a hospitable gut environment for long-term survival, reproduction and healthy function of essential microbes therein.

Enteric microbes that provide critical or essential contributions to mammalian host biology, whose genetics and life-cycles have co-evolved with their hosts over the course of millennia, can be referred to as “heirloom” taxa. These heirloom microbes often survive by a co-dependent relationship with their hosts, where host and microbe populations each afford essential survival advantages to one another.

For much of human evolutionary history, humans have been equipped from infancy onward with beneficial and even essential microbes, along with their “ex-genes” and encoded gene products, many of which are passed down reliably through generations via direct transmission. Vertically transmitted microbes in particular have evolved traits that directly improve human survival, and in this regard, the ex-genes of symbiotic microbes interact with human genes to form a “holobiont” unit of selection in evolution (see e.g., Rosenberg, E. & Zilber-Rosenberg, I. Symbiosis and development: the hologenome concept. Birth Defects Res C Embryo Today 93, 56-66). A clear manifestation of this co-evolutionary genetic model is provided by the immune system, which functions to coordinate transmission, expression, and silencing of microbial genes.

Where essential gene products are reliably afforded by a symbiotic gut microbe, fitness does not depend on having endogenous capacity to make those products. Where such endogenous capacities once existed but have become redundant due to exogenous substitution by microbes and their ex-genes, they will inevitably be lost over evolutionary time. Through normal mechanisms of mutation genes that no longer contribute significantly to fitness lose function over evolutionary time and become vestigial pseudogenes or are lost from the genome altogether.

Reliable intergenerational transmission of gut microbes and their ex-genes has allowed the mammalian microbiome to evolve as a community, contributing an integral, quasi-hereditary component to a “holobiont” evolutionary unit comprised of key heirloom microbe species and their co-evolved mammalian hosts. By virtue of this co-evolution, humans and other mammals exhibit complex interdependencies with their symbiotic gut microbes, involving genetic, metabolic, environmental and even host behavioral systems and processes, comparable to those of other ancient symbiotic partnerships (e.g., corals hosting endosymbiotic algae, hot-vent marine species hosting sulfur-fixing bacteria).

The most important result of mammalian co-evolution with symbiotic gut microbes is that many of the relationships humans share with these microbes have become obligate rather than facultative. Any substantial imbalance or impairment in the composition or function of the gut microbiome results in a “dysbiosis”, which can be accompanied by profound adverse health effects on the host. Only recently has western clinical medicine focused on the important health roles and functions of the natural gut microflora. Practical awareness is only now emerging that gut microbial dysbiosis is associated with a wide range of human diseases and adverse health conditions.

Extreme dysbiotic shifts in gut microbiome structure (including species composition, diversity, relative abundance and competition) and function (including structural, biochemical and metabolic activities) can be harmful and in severe cases devastating to host health. Such changes may be attributable to pronounced changes in the gut environment, for example caused by disease or external factors such as introduction of antibiotics, and may be exacerbated by competitive interactions between gut microfloral species (e.g., outgrowth, resource depletion, toxicity or other inhibitory effects mediated by other “pathogenic” bacterial or fungal species).

The most perilous conditions of human dysbiosis likely involve impairment or depletion of highly specialized microbes closely associated evolutionarily with human populations, particularly those heirloom species that are transmitted vertically and are difficult or impossible to acquire from the environment. While redundancies are expected to exist for many gut microfloral species that provide important ex-genes, metabolic pathways and products, certain microbial taxa may uniquely serve critical symbiotic roles, while others may be particularly susceptible to dysbiotic change (e.g., more vulnerable to elimination or competitive suppression in the wake of perturbations to the gut ecosystem). Most notably among these perturbations, antibiotic use, especially early in life or on the part of the mother before or during lactation, often results in profound and protracted changes in microbiome community structure and function. Differential antibiotic resistance among microbial taxa can further bias the microbiome toward dysbiosis, for example involving domination by a select few taxa, much like an invasive species in ecology. These and other dysbiotic influences can impose major limitations on the overall health and function of the gut microbiome, and when essential heirloom gut microbial species are negatively impacted the adverse impacts on their co-evolved, co-dependent mammalian host may be particularly dire.

In the emergent holistic medical field of gut microbiome ecology and related host health impacts, only general advances have been made to date. It is widely appreciated that dysbiosis impairing the gut microbiome can result in disruption or impairment of key microbial metabolic pathways and products important for host health and/or survival. Some of these pathways and products are implicated in diverse functions of the gut, including normal digestion and possibly regulation of gut-neural pathways. A related role for gut microbes in metabolic toxin clearance processes may exist. Other roles are proposed for gut microbes in supporting a healthy immune system, for cardiac disease prevention, to support healthy pulmonary and kidney function, and even for cancer prevention and elimination. Still other microbial genes, metabolic processes and products are suspected to support normal nervous system function, whereby deficits in these systems may be involved in impaired cognition, mood disorders and certain adverse mental health conditions.

Despite growing recognition in the medical community that the gut microbiome plays critical and diverse roles in human and veterinary health, little progress has been made to identify dietary and other factors affecting gut microfloral ecology, or to identify key microbial species, ex-genes, metabolic pathways and products at particular risk for dysbiosis that might impair or disrupt host health. Among the limited advances made to date, the medical community has recognized that widespread overuse of antibiotics should be curtailed, and adverse impacts of antibiotics on gut microfloral health should typically be mitigated by co-prescription of probiotics (ostensibly to re-colonize the gut with beneficial microbes following antibiotic-mediated crash of the gut microbiome). Additional focus has been afforded by medical researchers on gut microbiology prompted by increased prevalence of gut diseases such as Crohn's disease, inflammatory bowel syndrome and ulcerative colitis, leading to studies of dietary and other impacts on gut microbial ecology that may impact these diseases. But few answers have emerged from these studies, beyond a consensus that diets high in dairy, sugar, and processed foods contribute to higher incidence of these diseases, possibly based in part on adverse impacts on gut microfloral ecology.

In view of the foregoing, there remains a critical need in the medical arts for new tools and methods to diagnose and treat gut microbial dysbiosis in all its forms and clinical manifestations. A related need exists for more specific tools and methods to treat gut dysbiosis involving loss or impairment of key gut microbial species that contribute critical ex-genes, metabolic pathways and/or products essential for normal host health. More distinct and compelling needs exist to diagnose and treat specific dysbiosis-related health conditions, for example central nervous system (CNS) disorders caused or exacerbated by gut microbial dysbiosis affecting gut microbial taxa whose metabolism and/or metabolic products contribute to healthy CNS function.

### SUMMARY

The technology described herein fulfills the described needs and satisfies additional objects and advantages by providing compositions and methods for diagnosing, preventing and treating gut microbial dysbiosis, and/or directly treating or preventing clinical conditions arising from gut dysbiosis in mammals.

The technology described herein further provides compositions and methods for treating gut dysbiosis that features loss or impairment of microbial species expressing important ex-genes, metabolic pathways and products essential for normal host health, including normal central nervous system (CNS) function.

More detailed embodiments of the technology described herein provide active gut microbes, and probiotic compositions comprising these microbes, for administration to mammalian subjects, wherein the microbes are capable of stably colonizing the mammalian gastrointestinal (GI) tract and expressing ex-genes, metabolic pathways and products therein to correct dysbiosis and related adverse health impacts.

In exemplary embodiments, viable microbes (including natural or engineered bacterial strains) are delivered to a mammalian subject in dysbiosis, presenting a deficiency of one or more important gut bacteria, and/or of its expressed ex-genes, metabolic pathways or beneficial products involved in healthy host CNS function. Described herein is administration of such microbes to subjects presenting with an observed CNS disorder, such as a mood disorder, anxiety, autism or a mental health disorder like schizophrenia, to treat or prevent one or more symptoms of the CNS disorder.

In related embodiments, microbial metabolic precursors, enzymes, cofactors and/or metabolic products are delivered to a dysbiotic mammalian subject presenting with a CNS disorder, to replace or augment the function of a viable gut microbe expressing the subject precursors, enzymes, cofactors and/or products.

The technology described herein focuses in principal aspects on mammalian enteric microbes that are “heirloom” species strongly conserved across generations, on which the host relies symbiotically for metabolic pathways and products essential to normal host development and function. In certain aspects, the products of heirloom gut microfloral taxa of interest support healthy psychiatric and cognitive development and function.

In one exemplary embodiment, live microbes or their products employed within the methods and compositions of the technology described herein support the clearance of environmental neurotoxins, for example mercury or other heavy metals. These methods and compositions are effective to increase elimination rates of targeted compounds (e.g., toxins), and to effectively treat CNS disorders and other symptoms in subjects with dysbiosis involving loss or functional impairment of these detoxifying microbial strains. In illustrative embodiments, the methods and compositions of the technology described herein are used to substantially improve toxin clearance, including mercury clearance, within the gut and throughout the body, and to alleviate associated CNS disorders or symptoms.

Additional compositions and methods of the technology described herein employ an heirloom gut microbe, or compositions comprising products of these microbes, to treat dysbiosis affecting production and/or function of a neurotransmitter or neurotransmitter system in a mammalian host. These methods and compositions are effective to alleviate a diagnosed CNS disorder, for example a mood, attention, memory, or anxiety disorder, in subjects determined to be dysbiotic for one or more gut microbial species that synthesize products involved in neurotransmitter synthesis, or otherwise contribute to normal synthesis and/or function of neurotransmitters and/or neurotransmitter systems in their hosts. According to these aspects of the technology described herein, disruption of the microbiome, e.g., by disease or antibiotic use, leads to functional suppression or elimination of key microbial taxa that provide these functions, which leads to dysbiosis including abnormalities in a variety of psychiatric, neurodevelopmental, and neurodegenerative conditions of heretofore uncertain etiology. The methods and compositions of the technology described herein substantially improve neurotransmitter synthesis and/or function, and effectively prevent, treat or alleviate symptoms of the associated CNS disorder(s).

Other embodiments of the technology described herein employ heirloom gut microbes or compositions comprising products of these microbes to treat dysbiosis affecting queuine production and/or function in mammalian hosts, and to treat CNS disorders associated with loss or impairment of host queuine compounds and/or their precursors. Queuine is a modified nucleobase utilized by all eukaryotic organisms but produced exclusively by bacteria. Among the important activities described here for queuine, in regulating CNS function and mediating cognitive and mental health disorders in cases of queuine deficiency, queuine is involved in regenerating tetrahydrobiopterin (BH4) from its oxidation product dihydrobiopterin (BH2). BH4 is essential for the synthesis of the monoamine neurotransmitters serotonin, norepinephrine, dopamine, melatonin, and nitric oxide. Bacteria producing queuine or analogs thereof, or compositions comprising queuine or analogs thereof, can thus be used to treat dysbiosis affecting production and/or function of a neurotransmitter or neurotransmitter system in a mammalian host.

Other embodiments of the technology described herein employ heirloom gut microbes or compositions comprising products of these microbes to treat dysbiosis affecting endozepine production and/or function in mammalian hosts, and to treat CNS disorders associated with loss or impairment of host endozepine compounds and/or their synthetic precursors. Endozepines are naturally present in mammalian subjects, and are important CNS functional modulators mimicked by the anxiolytic drugs, benzodiazepines. Both groups of compounds act as positive allosteric modulators of gamma aminobutyric acid (GABA) receptor function to prevent or alleviate symptoms of anxiety or depression, and can thus be used to treat dysbiosis affecting production and/or function of a neurotransmitter or neurotransmitter system in a mammalian host.

Within exemplary embodiments of the technology described herein, methods and compositions are provided that alleviate a diagnosed CNS disorder, for example an anxiety disorder or depression, in subjects determined to be dysbiotic for one or more gut microbial species that synthesize products involved in queuine or endozepine synthesis, or otherwise contribute to normal synthesis and/or function of queuine or endozepines in their hosts. According to these aspects of the technology described herein, disruption of the microbiome leads to functional suppression or elimination of key microbial taxa that provide these functions, causing dysbiosis marked by loss or impairment of normal queuine or endozepine synthesis and/or function, and associated psychiatric, neurodevelopmental, and neurodegenerative conditions of heretofore uncertain etiology. The methods and compositions of the technology described herein substantially improve queuine or endozepine synthesis and/or function, and effectively prevent, treat or alleviate symptoms of the associated CNS disorder(s).

Within more detailed aspects of the technology described herein, selected heirloom bacterial species are identified, isolated, prepared and/or formulated for improved delivery, and administered to a dysbiotic mammalian subject to treat or prevent one or more CNS conditions or other symptom(s) attributable to the dysbiosis. The bacterial species are typically viable in a gut environment of treated subjects, however in other aspects non-viable (e.g., heat-killed) bacteria, or their isolated cellular contents, purified metabolic precursors, intermediates or products, can be effectively administered directly to address the dysbiosis and achieve the desired clinical benefits. Thus, in exemplary embodiments the bacterial species may be in the form of a live bacterial population, a lyophilized (e.g., viable) bacterial population, a non-viable bacterial preparation, or cellular components thereof (which may include metabolic precursors, intermediates or products of the subject bacteria, as well as synthetically derived replicates or chemically modified analogs thereof). Preferably, where the bacterial species is provided as a non-viable bacterial preparation, it is selected from heat-killed bacteria, irradiated bacteria and lysed bacteria.

In more detailed embodiments, the compositions and methods of the technology described herein may employ admixing of useful bacterial species, live or non-viable, or their isolated components, metabolic precursors, intermediates or products, with a pharmaceutically acceptable excipient, carrier, diluent or other agent that enhances delivery or activity of the administered composition. In some embodiments, the composition further comprises an enteric coating or similar composition to promote survival of or avoid the acidity of the stomach and permit delivery into the small or large intestines.

In related aspects, the technology described herein provides bacterial species and compositions comprising them in the form of “probiotics”, which are effective to improve intestinal microbial ecology, alleviate symptoms of microbial dysbiosis, and/or treat or prevent a CNS disorder in a mammalian subject.

Other aspects of the technology described herein relate to the use of gut bacterial species (live or non-viable), or metabolic precursors, intermediates or products thereof, in the preparation of medicaments for treating dysbiosis and associated CNS disorders and other adverse symptoms in mammalian subjects.

Other aspects of the technology described herein relate to the use of gut bacterial species for preparation of nutritional supplements or foodstuffs comprising the subject bacteria and optionally other ingredients that are generally accepted (or recognized) as safe for human consumption (“GRAS”), useful to support healthy gut ecology and normal CNS function in mammalian subjects.

Accordingly, in one aspect described herein is a composition comprising one or more isolated, non-pathogenic queuine-producing bacterial strains or an isolated product derived therefrom.

In some embodiments of any of the aspects, the one or more isolated, non-pathogenic queuine-producing bacterial strains comprise live bacteria or dead bacteria, or wherein the isolated product derived therefrom comprises culture medium in which said one or more isolated, non-pathogenic bacterial strains have been cultured.

In some embodiments of any of the aspects, the isolated product derived therefrom comprises a purified polypeptide produced by the one or more bacterial strains.

In some embodiments of any of the aspects, the composition further comprises a pharmaceutically acceptable carrier, wherein the one or more isolated non-pathogenic queuine-producing bacterial strains or an isolated product derived therefrom is present in an amount effective to alter queuine levels in a subject in need thereof.

In another aspect described herein is a pharmaceutical composition comprising queuine, an analog, derivative or precursor thereof, or a combination of any of these, in an amount effective to alter queuine levels in a subject in need thereof, and a pharmaceutically acceptable carrier.

In some embodiments of any of the aspects, the queuine, analog, derivative or precursor is isolated from a queuine-producing bacterial strain or culture medium in which a queuine-producing bacterial strain has been cultured.

In some embodiments of any of the aspects, the at least one isolated non-pathogenic queuine producing bacteria is a human gut bacteria.

In some embodiments of any of the aspects, the at least one isolated non-pathogenic queuine-producing bacteria belongs to a species selected from Acetobacter pasteurianus, Achromobacter xylosoxidans, Acidaminococcus fermentans, Acidaminococcus intestini, Acinetobacter baumannii, Acinetobacter calcoaceticus, Acinetobacter junii, Acinetobacter lwoffii, Acinetobacter pittii, Acinetobacter radioresistens, Acinetobacter schindleri, Acinetobacter towneri, Acinetobacter ursingii, Acinetobacter variabilis, Adlercreutzia equolifaciens, Aeribacillus pallidus, Aeromonas caviae, Aeromonas enteropelogenes, Aeromonas hydrophila, Aeromonas jandaei, Aeromonas salmonicida, Aeromonas schubertii, Aeromonas veronii, Aggregatibacter aphrophilus, Akkermansia muciniphila, Alistipes onderdonkii, Alistipes putredinis, Allisonella histaminiformans, Anaeroglobus geminatus, Anaerostipes caccae, Anaerostipes hadrus, Aneurinibacillus aneurinilyticus, Aneurinibacillus migulanus, Anoxybacillus flavithermus, Asaccharobacter celatus, Bacillus altitudinis, Bacillus amyloliquefaciens, Bacillus aquimaris, Bacillus atrophaeus, Bacillus badius, Bacillus bataviensis, Bacillus cereus, Bacillus circulans, Bacillus clausii, Bacillus coagulans, Bacillus cohnii, Bacillus endophyticus, Bacillus firmus, Bacillus flexus, Bacillus fordii, Bacillus galactosidilyticus, Bacillus halodurans, Bacillus infantis, Bacillus koreensis, Bacillus kyonggiensis, Bacillus lentus, Bacillus licheniformis, Bacillus litoralis, Bacillus marisflavi, Bacillus megaterium, Bacillus mojavensis, Bacillus mycoides, Bacillus nealsonii, Bacillus okuhidensis, Bacillus pseudofirmus, Bacillus pseudomycoides, Bacillus pumilus, Bacillus simplex, Bacillus sonorensis, Bacillus subterraneus, Bacillus subtilis, Bacillus thuringiensis, Bacillus timonensis, Bacillus vallismortis, Bacillus vietnamensis, Bacillus weihenstephanensis, Bacteroides caccae, Bacteroides cellulosilyticus, Bacteroides clarus, Bacteroides coprocola, Bacteroides dorei, Bacteroides eggerthii, Bacteroides faecis, Bacteroides fragilis, Bacteroides intestinalis, Bacteroides massiliensis, Bacteroides nordii, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bacteroides xylanolyticus, Barnesiella intestinihominis, Barnesiella viscericola, Bilophila wadsworthia, Blautia luti, Bordetella bronchiseptica, Bordetella trematum, Brenneria alni, Brevibacillus agri, Brevibacillus brevis, Brevibacillus choshinensis, Brevibacillus formosus, Brevibacillus laterosporus, Brevibacillus parabrevis, Brevundimonas diminuta, Butyricimonas virosa, Campylobacter coli, Campylobacter concisus, Campylobacter curvus, Campylobacter gracilis, Campylobacter jejuni, Campylobacter showae, Campylobacter ureolyticus, Cedecea lapagei, Cedecea neteri, Chromohalobacter japonicus, Citrobacter amalonaticus, Citrobacter braakii, Citrobacter farmeri, Citrobacter freundii, Citrobacter gillenii, Citrobacter koseri, Citrobacter murliniae, Citrobacter youngae, Clostridium acetireducens, Clostridium bartlettii, Clostridium beijerinckii, Clostridium botulinum, Clostridium butyricum, Clostridium carboxidivorans, Clostridium colicanis, Clostridium diolis, Clostridium disporicum, Clostridium novyi, Clostridium ramosum, Clostridium sporogenes, Clostridium thermocellum, Coprococcus catus, Coprococcus eutactus, Cronobacter sakazakii, Delftia tsuruhatensis, Desulfovibrio desulfuricans, Desulfovibrio fairfieldensis, Desulfovibrio piger, Dialister invisus, Dialister pneumosintes, Enterobacter aerogenes, Enterobacter asburiae, Enterobacter cloacae, Enterobacter hormaechei, Enterobacter kobei, Enterobacter ludwigii, Enterorhabdus caecimuris, Erysipelatoclostridium ramosum, Escherichia coli, Escherichiafergusonii, Escherichia hermannii, Escherichia marmotae, Geobacillus stearothermophilus, Haemophilus influenzae, Haemophilus pittmaniae, Hafnia alvei, Halobacillus dabanensis, Halobacillus karajensis, Halobacillus salinus, Halobacillus trueperi, Helicobacter pylori, Intestinibacter bartlettii, Klebsiella oxytoca, Klebsiella pneumoniae, Klebsiella variicola, Kluyvera cryocrescens, Kluyvera georgiana, Kosakonia cowanii, Kushneria sinocarnis, Lachnospira pectinoschiza, Lachnotalea glycerini, Lactobacillus mali, Leclercia adecarboxylata, Lelliottia amnigena, Litorilituus sediminis, Lysinibacillus boronitolerans, Lysinibacillus fusiformis, Lysinibacillus massiliensis, Lysinibacillus sphaericus, Lysinibacillus xylanilyticus, Lysobacter soli, Megasphaera elsdenii, Megasphaera micronuciformis, Micrococcus lylae, Mitsuokella jalaludinii, Moellerella wisconsensis, Monoglobus pectinilyticus, Moraxella osloensis, Morganella morganii, Neisseria canis, Neisseria cinerea, Neisseria elongata, Neisseria flavescens, Neisseria gonorrhoeae, Neisseria macacae, Neisseria meningitidis, Neisseria mucosa, Neisseria perflava, Neisseria subflava, Nosocomiicoccus massiliensis, Noviherbaspirillum denitrificans, Oceanobacillus iheyensis, Oceanobacillus oncorhynchi, Oceanobacillus sojae, Ochrobactrum anthropi, Odoribacter splanchnicus, Oxalobacter formigenes, Paenibacillus alvei, Paenibacillus amylolyticus, Paenibacillus barcinonensis, Paenibacillus barengoltzii, Paenibacillus daejeonensis, Paenibacillus dendritiformis, Paenibacillus glucanolyticus, Paenibacillus illinoisensis, Paenibacillus lactis, Paenibacillus larvae, Paenibacillus lautus, Paenibacillus macerans, Paenibacillus naphthalenovorans, Paenibacillus odorifer, Paenibacillus pabuli, Paenibacillus pasadenensis, Paenibacillus polymyxa, Paenibacillus rhizosphaerae, Paenibacillus stellifer, Paenibacillus thiaminolyticus, Paenibacillus typhae, Pantoea agglomerans, Parabacteroides distasonis, Parabacteroides goldsteinii, Parabacteroides gordonii, Parabacteroides johnsonii, Parabacteroides merdae, Paraprevotella clara, Parasutterella excrementihominis, Peptoniphilus asaccharolyticus, Peptoniphilus indolicus, Planococcus rifietoensis, Porphyromonas asaccharolytica, Porphyromonas bennonis, Porphyromonas somerae, Prevotella bivia, Prevotella buccae, Prevotella buccalis, Prevotella copri, Prevotella timonensis, Proteus mirabilis, Proteus penneri, Proteus vulgaris, Providencia alcalifaciens, Providencia heimbachae, Providencia rettgeri, Providencia stuartii, Pseudomonas aeruginosa, Pseudomonas alcaligenes, Pseudomonas bauzanensis, Pseudomonas caricapapayae, Pseudomonas chlororaphis, Pseudomonas fluorescens, Pseudomonas fragi, Pseudomonas fulva, Pseudomonas gessardii, Pseudomonas japonica, Pseudomonas libanensis, Pseudomonas lundensis, Pseudomonas luteola, Pseudomonas migulae, Pseudomonas monteilii, Pseudomonas mosselii, Pseudomonas oleovorans, Pseudomonas oryzihabitans, Pseudomonas putida, Pseudomonas rhodesiae, Pseudomonas saudiphocaensis, Pseudomonas stutzeri, Pseudomonas taetrolens, Pseudomonas tolaasii, Pseudomonas xanthomarina, Psychrobacter phenylpyruvicus, Raoultella ornithinolytica, Raoultella planticola, Roseomonas gilardii, Roseomonas mucosa, Ruminococcus albus, Ruminococcus callidus, Ruminococcus flavefaciens, Ruminococcus lactaris, Ruminococcus torques, Salinisphaera halophila, Salinivibrio costicola, Salmonella enterica, Salmonella enteritidis, Salmonella typhi, Selenomonas ruminantium, Selenomonas sputigena, Senegalimassilia anaerobia, Serratia marcescens, Serratia ureilytica, Shewanella xiamenensis, Shigella boydii, Shigella dysenteriae, Shigella flexneri, Shigella sonnei, Sphingomonas aerolata, Staphylococcus arlettae, Staphylococcus aureus, Staphylococcus auricularis, Staphylococcus capitis, Staphylococcus caprae, Staphylococcus carnosus, Staphylococcus cohnii, Staphylococcus condimenti, Staphylococcus devriesei, Staphylococcus epidermidis, Staphylococcus equorum, Staphylococcus gallinarum, Staphylococcus haemolyticus, Staphylococcus hominis, Staphylococcus hyicus, Staphylococcus intermedius, Staphylococcus kloosii, Staphylococcus lentus, Staphylococcus lugdunensis, Staphylococcus nepalensis, Staphylococcus pasteuri, Staphylococcus petrasii, Staphylococcus pettenkoferi, Staphylococcus saccharolyticus, Staphylococcus saprophyticus, Staphylococcus schleiferi, Staphylococcus sciuri, Staphylococcus simiae, Staphylococcus simulans, Staphylococcus succinus, Staphylococcus vitulinus, Staphylococcus warneri, Staphylococcus xylosus, Stenotrophomonas acidaminiphila, Stenotrophomonas maltophilia, Stenotrophomonas rhizophila, Streptococcus australis, Streptococcus bovis, Streptococcus equinus, Streptococcus gallolyticus, Streptococcus infantarius, Streptococcus infantis, Streptococcus lutetiensis, Streptococcus mitis, Streptococcus mutans, Streptococcus oralis, Streptococcus peroris, Streptococcus pseudopneumoniae, Streptococcus salivarius, Streptococcus sobrinus, Streptococcus thermophilus, Streptococcus tigurinus, Streptococcus vestibularis, Succiniclasticum ruminis, Terribacillus aidingensis, Terribacillus halophilus, Thermotalea metallivorans, Turicibacter sanguinis, Veillonella atypica, Veillonella denticariosi, Veillonella dispar, Veillonella parvula, Vibrio cholerae, Victivallis vadensis, Virgibacillus massiliensis, Yersinia bercovieri, Yersinia enterocolitica, Yersinia intermedia, Yersinia kristensenii, Yersinia mollaretii, and combinations thereof.

In some embodiments of any of the aspects, the one or more non-pathogenic queuine producing bacteria is a human gut bacteria, and comprises a 16S rRNA sequence at least about 97% identical to a 16S rRNA sequence selected from SEQ ID NOs 1-406.

In some embodiments of any of the aspects, the at least one isolated non-pathogenic queuine producing bacteria is a human gut bacteria that encodes within its genome and expresses in the human gastrointestinal tract at least one queuine biosynthesis enzyme selected from folE (GTP cyclohydrolase), QueD (6-carboxy-5,6,7,8-tetrahydrobiopterin synthase), QueE (7-carboxy-7-deazaguanine synthase), QueC (7-cyano-7-deazaguanine synthase, PreQ0 synthase), QueF (7-cyano-7-deazaguanine reductase, PreQ0 reductase), tgt or btgt (tRNA guanine transglycosylase, bacterial tRNA guanine transglycosylase), QueA (S-adenosylmethionine:tRNA ribosyltransferase-isomerase), and QueG or QueH (epoxyqueuosine reductase).

In some embodiments of any of the aspects, the at least one isolated non-pathogenic queuine producing bacteria is a human gut bacteria that encodes within its genome and expresses in the human gastrointestinal tract at least one queuine biosynthesis enzyme, wherein the amino acid sequence encoded by the at least one queuine biosynthesis gene is at least 90% similar to a sequence selected from SEQ ID NOs 3660-82283.

In some embodiments of any of the aspects, the at least one isolated non-pathogenic queuine producing bacteria is a human gut bacteria belongs to species selected from Acidaminococcus fermentans, Adlercreutzia equolifaciens, Akkermansia muciniphila, Alloprevotella tannerae, Anaerostipes caccae, Anaerostipes hadrus, Arcobacter butzleri, Bacteroides caccae, Bacteroides cellulosilyticus, Bacteroides clarus, Bacteroides coprophilus, Bacteroides dorei, Bacteroides eggerthii, Bacteroides faecis, Bacteroides fragilis, Bacteroides massiliensis, Bacteroides nordii, Bacteroides oleiciplenus, Bacteroides ovatus, Bacteroides plebeius, Bacteroides salanitronis, Bacteroides salyersiae, Bacteroides stercoris, Bacteroides thetaiotaomicron, Bacteroides uniformis, Bacteroides vulgatus, Bacteroides xylanisolvens, Barnesiella intestinihominis, Bilophila wadsworthia, Butyrivibrio crossotus, Campylobacter curvus, Citrobacter freundii, Citrobacter koseri, Clostridium bartelettii, Clostridium ramosum, Coprobacter fastidiosus, Coprococcus catus, Coprococcus eutactus, Desulfovibrio piger, Dialister invisus, Dialister succinatiphilus, Enterobacter aerogenes, Enterobacter cancerogenus, Enterobacter cloacae, Enterorhabdus caecimuris, Escherichia coli, Eubacterium hallii, Fusobacterium mortiferum, Haemophilus pittmaniae, Haemophilus sputorum, Hafnia alvei, Klebsiella oxytoca, Klebsiella pneumoniae, Klebsiella variicola, Megamonas funiformis, Megamonas rupellensis, Megasphaera elsdenii, Megasphaera micronuciformis, Mitsuokella multacida, Odoribacter laneus, Odoribacter splanchnicus, Oxalobacter formigenes, Parabacteroides distasonis, Porphyromonas asaccharolytica, Porphyromonas uenonis, Ruminococcus callidus, Ruminococcus torques, Shigella sonnei, Streptococcus infantis, Streptococcus mitis, Streptococcus oralis, Streptococcus pneumoniae, Streptococcus tigurinus, Turicibacter sanguinis, Veillonella atypica, Veillonella dispar, Veillonella parvula, Dysgonomonas mossii, Proteus mirabilis, Veillonella ratti, and combinations thereof, and wherein the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.

In some embodiments of any of the aspects, the at least one isolated non-pathogenic queuine producing bacteria is a human gut bacteria with a 16S rRNA sequence at least about 97% identical to a 16S rRNA sequence selected from SEQ ID NOs 1-78, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.

In some embodiments of any of the aspects, the queuine precursor is epoxyqueuine and/or cobalamin.

In some embodiments of any of the aspects, the queuine analogs are selected from queuosine, a mannosyl queuosine, galactosyl queuosine, glutamyl queuosine, mannosylqueuine, galactosylqueuine, and aminoacylated derivatives such as glutamylqueuine.

In some embodiments of any of the aspects, the composition is formulated in a capsule, a tablet, a caplet, a pill, a troche, a lozenge, a powder, a granule, a nutraceutical, a medical food, or a combination thereof.

In some embodiments of any of the aspects, the composition is formulated for delivery to the gut.

In some embodiments of any of the aspects, the composition further comprises a prebiotic.

In some embodiments of any of the aspects, the composition further comprises a different composition in an amount effective to treat a CNS disease or disorder.

In some embodiments of any of the aspects, the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or combinations thereof, and formulated for delivery with a pharmaceutically acceptable excipient, carrier or diluent.

In another aspect described herein is a method of increasing queuine levels in a subject in need thereof, the method comprising administering to the subject a composition as described herein in an amount effective to increase queuine levels in the subject.

In some embodiments of any of the aspects, the subject is a mammalian subject.

In some embodiments of any of the aspects, the subject is a human subject.

In another aspect described herein is a method for treating or preventing a gut microbiome dysbiosis-mediated central nervous system (CNS) disorder associated with queuine deficiency in a mammalian subject in need thereof, comprising administering to a subject dysbiotic for queuine producing gut microbes or low in queuine one or more isolated queuine-producing bacterial strains or an isolated product derived therefrom in an amount sufficient to increase queuine or to establish a queuine level within the range of normal in the subject, whereby one or more symptoms of the CNS disorder associated with queuine deficiency in the subject is improved.

In another aspect described herein is a method for treating or preventing a central nervous system (CNS) disorder associated with queuine deficiency in a mammalian subject in need thereof, comprising administering to the subject a composition comprising an agent selected from queuine, a queuine precursor, or a queuine analog, in an amount sufficient to increase queuine or to establish a queuine level within the range of normal in the subject, whereby one or more symptoms of the CNS disorder associated with queuine deficiency in the subject is improved.

In some embodiments of any of the aspects, the CNS disorder is selected from a cognitive disorder, a mood disorder, an anxiety disorder, and a psychiatric disorder.

In some embodiments of any of the aspects, the CNS disorder is selected from autism, bipolar disorder, major depression, anxiety and schizophrenia.

In some embodiments of any of the aspects, the method further comprises identifying a subject in need of treatment by determining whether the subject would benefit from an increase in endogenous queuine.

In some embodiments of any of the aspects, the amount of queuine in the subject's blood, liver, brain, serum, or stool is below 50 ng/mL.

In some embodiments of any of the aspects, the amount of queuosine-modified Histidyl tRNA in a sample of the subject's blood, liver, brain, serum, or stool is less than 80% that of the total Histidyl tRNA in the sample.

In some embodiments of any of the aspects, the amount of queuine-producing bacteria in the subject's stool is less than about 10% of total bacteria as measured by 16S sequence or shotgun sequencing.

In some embodiments of any of the aspects, the amount of queuine, queuine-incorporated RNA, or BH4 in the subject's blood, liver, brain, serum, or stool is increased relative to the initial amount after administering the composition.

In some embodiments of any of the aspects, the amount of queuine producing bacteria is increased in the subject's stool relative to the initial amount after administering the composition.

In some embodiments of any of the aspects, the amount of queuine producing genes are increased in the subject's stool relative to the initial amount after administering the composition.

In some embodiments of any of the aspects, the at least one isolated non-pathogenic queuine producing bacteria is a human gut bacteria, and belongs to the species selected from Acetobacter pasteurianus, Achromobacter xylosoxidans, Acidaminococcus fermentans, Acidaminococcus intestini, Acinetobacter baumannii, Acinetobacter calcoaceticus, Acinetobacter junii, Acinetobacter lwoffii, Acinetobacter pittii, Acinetobacter radioresistens, Acinetobacter schindleri, Acinetobacter towneri, Acinetobacter ursingii, Acinetobacter variabilis, Adlercreutzia equolifaciens, Aeribacillus pallidus, Aeromonas caviae, Aeromonas enteropelogenes, Aeromonas hydrophila, Aeromonas jandaei, Aeromonas salmonicida, Aeromonas schubertii, Aeromonas veronii, Aggregatibacter aphrophilus, Akkermansia muciniphila, Alistipes onderdonkii, Alistipes putredinis, Allisonella histaminiformans, Anaeroglobus geminatus, Anaerostipes caccae, Anaerostipes hadrus, Aneurinibacillus aneurinilyticus, Aneurinibacillus migulanus, Anoxybacillus flavithermus, Asaccharobacter celatus, Bacillus altitudinis, Bacillus amyloliquefaciens, Bacillus aquimaris, Bacillus atrophaeus, Bacillus badius, Bacillus bataviensis, Bacillus cereus, Bacillus circulans, Bacillus clausii, Bacillus coagulans, Bacillus cohnii, Bacillus endophyticus, Bacillus firmus, Bacillus flexus, Bacillus fordii, Bacillus galactosidilyticus, Bacillus halodurans, Bacillus infantis, Bacillus koreensis, Bacillus kyonggiensis, Bacillus lentus, Bacillus licheniformis, Bacillus litoralis, Bacillus marisflavi, Bacillus megaterium, Bacillus mojavensis, Bacillus mycoides, Bacillus nealsonii, Bacillus okuhidensis, Bacillus pseudofirmus, Bacillus pseudomycoides, Bacillus pumilus, Bacillus simplex, Bacillus sonorensis, Bacillus subterraneus, Bacillus subtilis, Bacillus thuringiensis, Bacillus timonensis, Bacillus vallismortis, Bacillus vietnamensis, Bacillus weihenstephanensis, Bacteroides caccae, Bacteroides cellulosilyticus, Bacteroides clarus, Bacteroides coprocola, Bacteroides dorei, Bacteroides eggerthii, Bacteroides faecis, Bacteroides fragilis, Bacteroides intestinalis, Bacteroides massiliensis, Bacteroides nordii, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bacteroides xylanolyticus, Barnesiella intestinihominis, Barnesiella viscericola, Bilophila wadsworthia, Blautia luti, Bordetella bronchiseptica, Bordetella trematum, Brenneria alni, Brevibacillus agri, Brevibacillus brevis, Brevibacillus choshinensis, Brevibacillus formosus, Brevibacillus laterosporus, Brevibacillus parabrevis, Brevundimonas diminuta, Butyricimonas virosa, Campylobacter coli, Campylobacter concisus, Campylobacter curvus, Campylobacter gracilis, Campylobacter jejuni, Campylobacter showae, Campylobacter ureolyticus, Cedecea lapagei, Cedecea neteri, Chromohalobacter japonicus, Citrobacter amalonaticus, Citrobacter braakii, Citrobacter farmeri, Citrobacter freundii, Citrobacter gillenii, Citrobacter koseri, Citrobacter murliniae, Citrobacter youngae, Clostridium acetireducens, Clostridium bartlettii, Clostridium beijerinckii, Clostridium botulinum, Clostridium butyricum, Clostridium carboxidivorans, Clostridium colicanis, Clostridium diolis, Clostridium disporicum, Clostridium novyi, Clostridium ramosum, Clostridium sporogenes, Clostridium thermocellum, Coprococcus catus, Coprococcus eutactus, Cronobacter sakazakii, Delftia tsuruhatensis, Desulfovibrio desulfuricans, Desulfovibrio fairfieldensis, Desulfovibrio piger, Dialister invisus, Dialister pneumosintes, Enterobacter aerogenes, Enterobacter asburiae, Enterobacter cloacae, Enterobacter hormaechei, Enterobacter kobei, Enterobacter ludwigii, Enterorhabdus caecimuris, Erysipelatoclostridium ramosum, Escherichia coli, Escherichia fergusonii, Escherichia hermannii, Escherichia marmotae, Geobacillus stearothermophilus, Haemophilus influenzae, Haemophilus pittmaniae, Hafnia alvei, Halobacillus dabanensis, Halobacillus karajensis, Halobacillus salinus, Halobacillus trueperi, Helicobacter pylori, Intestinibacter bartlettii, Klebsiella oxytoca, Klebsiella pneumoniae, Klebsiella variicola, Kluyvera cryocrescens, Kluyvera georgiana, Kosakonia cowanii, Kushneria sinocarnis, Lachnospira pectinoschiza, Lachnotalea glycerini, Lactobacillus mali, Leclercia adecarboxylata, Lelliottia amnigena, Litorilituus sediminis, Lysinibacillus boronitolerans, Lysinibacillus fusiformis, Lysinibacillus massiliensis, Lysinibacillus sphaericus, Lysinibacillus xylanilyticus, Lysobacter soli, Megasphaera elsdenii, Megasphaera micronuciformis, Micrococcus lylae, Mitsuokella jalaludinii, Moellerella wisconsensis, Monoglobus pectinilyticus, Moraxella osloensis, Morganella morganii, Neisseria canis, Neisseria cinerea, Neisseria elongata, Neisseria flavescens, Neisseria gonorrhoeae, Neisseria macacae, Neisseria meningitidis, Neisseria mucosa, Neisseria perflava, Neisseria subflava, Nosocomiicoccus massiliensis, Noviherbaspirillum denitrificans, Oceanobacillus iheyensis, Oceanobacillus oncorhynchi, Oceanobacillus sojae, Ochrobactrum anthropi, Odoribacter splanchnicus, Oxalobacter formigenes, Paenibacillus alvei, Paenibacillus amylolyticus, Paenibacillus barcinonensis, Paenibacillus barengoltzii, Paenibacillus daejeonensis, Paenibacillus dendritiformis, Paenibacillus glucanolyticus, Paenibacillus illinoisensis, Paenibacillus lactis, Paenibacillus larvae, Paenibacillus lautus, Paenibacillus macerans, Paenibacillus naphthalenovorans, Paenibacillus odorifer, Paenibacillus pabuli, Paenibacillus pasadenensis, Paenibacillus polymyxa, Paenibacillus rhizosphaerae, Paenibacillus stellifer, Paenibacillus thiaminolyticus, Paenibacillus typhae, Pantoea agglomerans, Parabacteroides distasonis, Parabacteroides goldsteinii, Parabacteroides gordonii, Parabacteroides johnsonii, Parabacteroides merdae, Paraprevotella clara, Parasutterella excrementihominis, Peptoniphilus asaccharolyticus, Peptoniphilus indolicus, Planococcus rifetoensis, Porphyromonas asaccharolytica, Porphyromonas bennonis, Porphyromonas somerae, Prevotella bivia, Prevotella buccae, Prevotella buccalis, Prevotella copri, Prevotella timonensis, Proteus mirabilis, Proteus penneri, Proteus vulgaris, Providencia alcalifaciens, Providencia heimbachae, Providencia rettgeri, Providencia stuartii, Pseudomonas aeruginosa, Pseudomonas alcaligenes, Pseudomonas bauzanensis, Pseudomonas caricapapayae, Pseudomonas chlororaphis, Pseudomonas fluorescens, Pseudomonas fragi, Pseudomonas fulva, Pseudomonas gessardii, Pseudomonas japonica, Pseudomonas libanensis, Pseudomonas lundensis, Pseudomonas luteola, Pseudomonas migulae, Pseudomonas monteilii, Pseudomonas mosselii, Pseudomonas oleovorans, Pseudomonas oryzihabitans, Pseudomonas putida, Pseudomonas rhodesiae, Pseudomonas saudiphocaensis, Pseudomonas stutzeri, Pseudomonas taetrolens, Pseudomonas tolaasii, Pseudomonas xanthomarina, Psychrobacter phenylpyruvicus, Raoultella ornithinolytica, Raoultella planticola, Roseomonas gilardii, Roseomonas mucosa, Ruminococcus albus, Ruminococcus callidus, Ruminococcus flavefaciens, Ruminococcus lactaris, Ruminococcus torques, Salinisphaera halophila, Salinivibrio costicola, Salmonella enterica, Salmonella enteritidis, Salmonella typhi, Selenomonas ruminantium, Selenomonas sputigena, Senegalimassilia anaerobia, Serratia marcescens, Serratia ureilytica, Shewanella xiamenensis, Shigella boydii, Shigella dysenteriae, Shigella flexneri, Shigella sonnei, Sphingomonas aerolata, Staphylococcus arlettae, Staphylococcus aureus, Staphylococcus auricularis, Staphylococcus capitis, Staphylococcus caprae, Staphylococcus carnosus, Staphylococcus cohnii, Staphylococcus condimenti, Staphylococcus devriesei, Staphylococcus epidermidis, Staphylococcus equorum, Staphylococcus gallinarum, Staphylococcus haemolyticus, Staphylococcus hominis, Staphylococcus hyicus, Staphylococcus intermedius, Staphylococcus kloosii, Staphylococcus lentus, Staphylococcus lugdunensis, Staphylococcus nepalensis, Staphylococcus pasteuri, Staphylococcus petrasii, Staphylococcus pettenkoferi, Staphylococcus saccharolyticus, Staphylococcus saprophyticus, Staphylococcus schleiferi, Staphylococcus sciuri, Staphylococcus simiae, Staphylococcus simulans, Staphylococcus succinus, Staphylococcus vitulinus, Staphylococcus warneri, Staphylococcus xylosus, Stenotrophomonas acidaminiphila, Stenotrophomonas maltophilia, Stenotrophomonas rhizophila, Streptococcus australis, Streptococcus bovis, Streptococcus equinus, Streptococcus gallolyticus, Streptococcus infantarius, Streptococcus infantis, Streptococcus lutetiensis, Streptococcus mitis, Streptococcus mutans, Streptococcus oralis, Streptococcus peroris, Streptococcus pseudopneumoniae, Streptococcus salivarius, Streptococcus sobrinus, Streptococcus thermophilus, Streptococcus tigurinus, Streptococcus vestibularis, Succiniclasticum ruminis, Terribacillus aidingensis, Terribacillus halophilus, Thermotalea metallivorans, Turicibacter sanguinis, Veillonella atypica, Veillonella denticariosi, Veillonella dispar, Veillonella parvula, Vibrio cholerae, Victivallis vadensis, Virgibacillus massiliensis, Yersinia bercovieri, Yersinia enterocolitica, Yersinia intermedia, Yersinia kristensenii, Yersinia mollaretii, and combinations thereof.

In some embodiments of any of the aspects, the one or more non-pathogenic queuine producing bacteria is a human gut bacteria, and consists of one or more bacteria comprising a 16S rRNA sequence at least about 97% identical to a 16S rRNA sequence selected from SEQ ID NOs 1-406.

In some embodiments of any of the aspects, the at least one isolated non-pathogenic queuine producing bacteria is a human gut bacteria that encodes within its genome and expresses in the human gastrointestinal tract at least one queuine biosynthesis selected from folE (GTP cyclohydrolase), QueD (6-carboxy-5,6,7,8-tetrahydrobiopterin synthase), QueE (7-carboxy-7-deazaguanine synthase), QueC (7-cyano-7-deazaguanine synthase, PreQ0 synthase), QueF (7-cyano-7-deazaguanine reductase, PreQ0 reductase), tgt or btgt (tRNA guanine transglycosylase, bacterial tRNA guanine transglycosylase), QueA (S-adenosylmethionine:tRNA ribosyltransferase-isomerase), and QueG or QueH (epoxyqueuosine reductase).

In some embodiments of any of the aspects, the at least one isolated non-pathogenic queuine producing bacteria is a human gut bacteria that encodes within its genome and expresses in the human gastrointestinal tract at least one queuine biosynthesis enzyme, wherein the amino acid sequence encoded by the at least one queuine biosynthesis gene is at least 90% similar to a sequence selected from SEQ ID NOs 3660-82283.

In some embodiments of any of the aspects, the at least one isolated non-pathogenic queuine producing bacteria is a human gut bacteria belongs to the species selected from Acidaminococcus fermentans, Adlercreutzia equolifaciens, Akkermansia muciniphila, Alloprevotella tannerae, Anaerostipes caccae, Anaerostipes hadrus, Arcobacter butzleri, Bacteroides caccae, Bacteroides cellulosilyticus, Bacteroides clarus, Bacteroides coprophilus, Bacteroides dorei, Bacteroides eggerthii, Bacteroides faecis, Bacteroides fragilis, Bacteroides massiliensis, Bacteroides nordii, Bacteroides oleiciplenus, Bacteroides ovatus, Bacteroides plebeius, Bacteroides salanitronis, Bacteroides salyersiae, Bacteroides stercoris, Bacteroides thetaiotaomicron, Bacteroides uniformis, Bacteroides vulgatus, Bacteroides xylanisolvens, Barnesiella intestinihominis, Bilophila wadsworthia, Butyrivibrio crossotus, Campylobacter curvus, Citrobacter freundii, Citrobacter koseri, Clostridium bartelettii, Clostridium ramosum, Coprobacter fastidiosus, Coprococcus catus, Coprococcus eutactus, Desulfovibrio piger, Dialister invisus, Dialister succinatiphilus, Enterobacter aerogenes, Enterobacter cancerogenus, Enterobacter cloacae, Enterorhabdus caecimuris, Escherichia coli, Eubacterium hallii, Fusobacterium mortiferum, Haemophilus pittmaniae, Haemophilus sputorum, Hafnia alvei, Klebsiella oxytoca, Klebsiella pneumoniae, Klebsiella variicola, Megamonas funiformis, Megamonas rupellensis, Megasphaera elsdenii, Megasphaera micronuciformis, Mitsuokella multacida, Odoribacter laneus, Odoribacter splanchnicus, Oxalobacter formigenes, Parabacteroides distasonis, Porphyromonas asaccharolytica, Porphyromonas uenonis, Ruminococcus callidus, Ruminococcus torques, Shigella sonnei, Streptococcus infantis, Streptococcus mitis, Streptococcus oralis, Streptococcus pneumoniae, Streptococcus tigurinus, Turicibacter sanguinis, Veillonella atypica, Veillonella dispar, Veillonella parvula, Dysgonomonas mossii, Proteus mirabilis, or Veillonella ratti, and combinations thereof, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.

In some embodiments of any of the aspects, the at least one isolated non-pathogenic queuine producing bacteria is a human gut bacteria with a 16S rRNA sequence at least about 97% identical to a 16S rRNA sequence selected from SEQ ID NOs 1-78, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.

In some embodiments of any of the aspects, the queuine precursors are selected from epoxyqueuine and/or cobalamin.

In some embodiments of any of the aspects, the queuine analogs are selected from queuosine, a mannosyl queuosine, galactosyl queuosine, glutamyl queuosine, mannosylqueuine, galactosylqueuine, and aminoacylated derivatives such as glutamylqueuine.

In some embodiments of any of the aspects, the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or combinations thereof, and formulated for delivery with a pharmaceutically acceptable excipient, carrier or diluent.

In some embodiments of any of the aspects, the administered composition is formulated in a capsule, a tablet, a caplet, a pill, a troche, a lozenge, a powder, a granule, nutraceutical, a medical food, or a combination thereof.

In some embodiments of any of the aspects, the composition is formulated for delivery to the gut.

In some embodiments of any of the aspects, the composition further comprises a prebiotic.

In some embodiments of any of the aspects, the composition further comprises a different composition in an amount effective to treat a CNS disease or disorder.

In another aspect, described herein is a composition comprising one or more isolated non-pathogenic endozepine-producing bacterial or yeast strains or an isolated product derived therefrom.

In some embodiments of any of the aspects, the one or more isolated, non-pathogenic endozepine-producing bacterial or yeast strains comprise live bacteria or yeast, or dead bacteria or yeast, or wherein the isolated product derived therefrom comprises culture medium in which said one or more isolated, non-pathogenic bacterial or yeast strains have been cultured.

In some embodiments of any of the aspects, the isolated product derived therefrom comprises a purified polypeptide produced by the one or more bacterial or yeast strains.

In some embodiments of any of the aspects, the composition further comprises a pharmaceutically acceptable carrier, wherein the one or more isolated non-pathogenic queuine-producing bacterial or yeast strains or an isolated product derived therefrom is present in an amount effective to alter endozepine levels in a subject in need thereof.

In another aspect described herein is a pharmaceutical composition comprising endozepine, an analog, derivative or precursor thereof, or a combination of any of these, in an amount effective to alter endozepine levels in a subject in need thereof, and a pharmaceutically acceptable carrier.

In some embodiments of any of the aspects, the endozepine analog, derivative or precursor is isolated from an endozepine-producing bacterial or yeast strain or culture medium in which an endozepine-producing bacterial or yeast strain has been cultured.

In another aspect described herein is a method of increasing endozepine levels in a subject in need thereof, the method comprising administering to the subject a composition as described herein in an amount effective to increase endozepine levels in the subject.

In some embodiments of any of the aspects, the subject is a mammalian subject.

In some embodiments of any of the aspects, the subject is a human subject.

In another aspect described herein is a method for treating or preventing a gut microbiome dysbiosis-mediated central nervous system (CNS) disorder associated with an endozepine deficiency in a mammalian subject in need thereof, comprising administering to a subject dysbiotic for endozepine producing gut microbes or low in endozepines one or more isolated non-pathogenic endozepine producing bacterial or yeast strains, an isolated product derived therefrom, endozepines, prebiotics, or combinations thereof, which alter endozepine levels in a subject in need thereof, wherein the composition is formulated for oral or intravenous delivery with a pharmaceutically acceptable excipient, carrier or diluent.

In some embodiments of any of the aspects, the one or more isolated non-pathogenic endozepine producing bacterial or yeast strains comprises live bacteria or yeast, dead bacteria or yeast, spent medium(s) derived from a bacteria or yeast, cell pellet(s) of a bacteria or yeast, purified metabolite(s) produced by bacteria or yeast, purified protein(s) produced by a bacteria or yeast, and combinations thereof.

In another aspect described herein is a composition comprising one or more isolated non-pathogenic heavy metal sequestering bacterial strains, their derivatives, siderophores, prebiotics, or combinations thereof, which alter heavy metal levels in a subject in need thereof, wherein the composition is formulated for oral or intravenous delivery with a pharmaceutically acceptable excipient, carrier or diluent.

In some embodiments of any of the aspects, the one or more isolated non-pathogenic heavy metal sequestering bacterial strains is a purified strain.

In some embodiments of any of the aspects, the one or more isolated non-pathogenic heavy metal sequestering bacterial strains comprises live bacteria, dead bacteria, spent medium(s) derived from a bacteria, cell pellet(s) of a bacteria, purified metabolite(s) produced by bacteria, purified protein(s) produced by a bacteria, and combinations thereof.

In another aspect described herein is a method for treating or preventing a gut microbiome dysbiosis-mediated central nervous system (CNS) disorder associated with a heavy metal toxicity in a mammalian subject in need thereof, comprising administering to subjects dysbiotic for heavy metal sequestering gut microbes or high in toxic heavy metals one or more isolated non-pathogenic heavy metal sequestering bacterial strains (e.g., purified strains), their derivatives (e.g. live bacteria, dead bacteria, spent medium(s) derived from a bacteria, cell pellet(s) of a bacteria, purified metabolite(s) produced by bacteria, purified protein(s) produced by a bacteria, or combinations thereof), siderophores, prebiotics, or combinations thereof, which alter endozepine levels in a subject in need thereof, wherein the composition is formulated for oral or intravenous delivery with a pharmaceutically acceptable excipient, carrier or diluent.

In some embodiments of any of the aspects, the one or more isolated non-pathogenic heavy metal sequestering bacterial strains is a purified strain.

In some embodiments of any of the aspects, the one or more isolated non-pathogenic heavy metal sequestering bacterial strains comprises live bacteria, dead bacteria, spent medium(s) derived from a bacteria, cell pellet(s) of a bacteria, purified metabolite(s) produced by bacteria, purified protein(s) produced by a bacteria, and combinations thereof.

In another aspect described herein is a method of increasing BH4 levels in a subject in need thereof, the method comprising administering to the subject a composition of any one of claims 1-20 in an amount effective to increase BH4 levels in the subject.

In some embodiments of any of the aspects, the subject is a mammalian subject.

In some embodiments of any of the aspects, the subject is a human subject.

In another aspect described herein is a composition as described herein, for use in treating a queuine-related CNS disease or disorder.

In some embodiments of any of the aspects, the CNS disease or disorder is selected from a cognitive disorder, a mood disorder, an anxiety disorder, and a psychiatric disorder.

In some embodiments of any of the aspects, the CNS disorder is selected from autism, bipolar disorder, major depression, anxiety and schizophrenia.

In some embodiments of any of the aspects, treating comprises administering the composition to an individual diagnosed as having a queuine-related CNS disease or disorder.

In some embodiments of any of the aspects, treating comprises, prior to administering the composition for use, identifying a subject in need of treatment by determining whether the subject would benefit from an increase in endogenous queuine.

In some embodiments of any of the aspects, identifying a subject in need comprises measurement of the amount of queuine in the subject's blood, liver, brain, serum or stool.

In some embodiments of any of the aspects, identifying a subject in need comprises measurement of queuosine-modified Histidyl-tRNA in a sample of the subject's blood, liver, brain, serum or stool.

In some embodiments of any of the aspects, identifying a subject in need comprises measurement of queuine-producing bacteria in the subject's stool by 16S rRNA sequencing.

In some embodiments of any of the aspects, the amount of queuine-producing bacteria in the subject's stool is less than about 10% of total bacteria as measured by 16S rRNA sequencing.

In another aspect described herein is use of a composition as described herein for the treatment of a queuine-related CNS disease or disorder.

In some embodiments of any of the aspects, the CNS disease or disorder is selected from a cognitive disorder, a mood disorder, an anxiety disorder, and a psychiatric disorder.

In some embodiments of any of the aspects, the CNS disorder is selected from autism, bipolar disorder, major depression, anxiety and schizophrenia.

In another aspect described herein is a composition as described herein, for use in treating a gut microbial dysbiosis.

In some embodiments of any of the aspects, the gut microbial dysbiosis comprises a deficiency in queuine-producing gut bacteria.

In some embodiments of any of the aspects, treating comprises administering the composition to an individual diagnosed as having a deficiency in queuine-producing gut bacteria.

In some embodiments of any of the aspects, treating comprises, prior to administering the composition for use, identifying a subject in need of treatment by determining that the subject has a deficiency in queuine-producing gut bacteria.

In some embodiments of any of the aspects, identifying a subject in need comprises measurement of the amount of queuine in the subject's blood, liver, brain, serum or stool.

In some embodiments of any of the aspects, identifying a subject in need comprises measurement of queuosine-modified Histidyl-tRNA in a sample of the subject's blood, liver, brain, serum or stool.

In some embodiments of any of the aspects, identifying a subject in need comprises measurement of queuine-producing bacteria in the subject's stool by 16S rRNA sequencing.

In some embodiments of any of the aspects, the amount of queuine-producing bacteria in the subject's stool is less than about 10% of total bacteria as measured by 16S rRNA sequencing.

In another aspect described herein is use of a composition as described herein for treating a gut microbial dysbiosis.

In some embodiments of any of the aspects, the gut microbial dysbiosis comprises a deficiency in queuine-producing gut bacteria.

In some embodiments of any of the aspects, treating comprises administering the composition to an individual diagnosed as having a deficiency in queuine-producing gut bacteria.

In some embodiments of any of the aspects, treating comprises, prior to administering the composition for use, identifying a subject in need of treatment by determining that the subject has a deficiency in queuine-producing gut bacteria.

In some embodiments of any of the aspects, identifying a subject in need comprises measurement of the amount of queuine in the subject's blood, liver, brain, serum or stool.

In some embodiments of any of the aspects, identifying a subject in need comprises measurement of queuosine-modified Histidyl-tRNA in a sample of the subject's blood, liver, brain, serum or stool.

In some embodiments of any of the aspects, identifying a subject in need comprises measurement of queuine-producing bacteria in the subject's stool by 16S rRNA sequencing.

In some embodiments of any of the aspects, the amount of queuine-producing bacteria in the subject's stool is less than about 10% of total bacteria as measured by 16S rRNA sequencing.

In another aspect described herein is a composition as described herein, for use in treating a BH4 deficiency or increasing the level of BH4 in a subject in need thereof.

In another aspect described herein is use of a composition as described herein, for treating a BH4 deficiency or increasing the level of BH4 in a subject in need thereof.

In another aspect described herein is a composition as described herein, for use in treating or preventing a gut microbiome dysbiosis-mediated central nervous system (CNS) disorder associated with an endozepine deficiency in a mammalian subject in need thereof.

In some embodiments of any of the aspects, the CNS disease or disorder is selected from a cognitive disorder, a mood disorder, an anxiety disorder, and a psychiatric disorder.

In some embodiments of any of the aspects, the CNS disorder is selected from autism, bipolar disorder, major depression, anxiety and schizophrenia.

In some embodiments of any of the aspects, treating comprises administering the composition to an individual diagnosed as having a gut microbiome dysbiosis-mediated central nervous system (CNS) disorder associated with an endozepine deficiency.

In some embodiments of any of the aspects, treating comprises, prior to administering the composition for use, identifying a subject in need of treatment by determining whether the subject would benefit from an increase in endogenous endozepine.

In some embodiments of any of the aspects, identifying a subject in need comprises measurement of the amount of endozepine in the subject's blood, liver, brain, serum or stool.

In another aspect described herein is use of a composition as described herein, for use in treating or preventing a gut microbiome dysbiosis-mediated central nervous system (CNS) disorder associated with an endozepine deficiency in a mammalian subject in need thereof.

In another aspect described herein is a composition as described herein, for use in treating or preventing a gut microbiome dysbiosis-mediated central nervous system (CNS) disorder associated with a heavy metal toxicity in a mammalian subject in need thereof.

In some embodiments of any of the aspects, treating comprises administering the composition to an individual diagnosed as having a gut microbiome dysbiosis-mediated central nervous system (CNS) disorder associated with a heavy metal toxicity.

In some embodiments of any of the aspects, treating comprises, prior to administering the composition for use, identifying a subject in need of treatment by determining whether the subject would benefit from a reduction in a heavy metal level.

In another aspect described herein use of a composition as described herein for the treatment or prevention of a gut microbiome dysbiosis-mediated central nervous system (CNS) disorder associated with a heavy metal toxicity.

### BRIEF DESCRIPTION OF THE DRAWINGS

FIG. 1A-1C is a schematic flow diagram illustrating the various metabolic pathways targeted within the technology described herein involved in central nervous system (CNS) function and mental health, to which critical species within the mammalian gut microbiome contribute important metabolic processes, products and functions. FIG. 1 is adapted from C. Fergus, et al. Nutrients 2015 April; 7(4): 2897-2929.

FIG. 2A-2B are schematic flow diagrams illustrating various metabolic pathways and components involved in neurotransmitter biology and related CNS function(s) targeted within the technology described herein.

FIG. 3 is a summary of one bacterial pathway involved in queuine biosynthesis.

FIG. 4 is a summary of some bacterial siderophore compounds with relevance to the technology described herein. FIG. 4 is based on a schematic from Kanehisa Laboratories© (01053, 3/11/26).

FIG. 5 details intraperitoneal administration of queuine to deficient animals; in FIG. 5, each queuine injection was 325 micrograms, administered to mice intraperitoneally 24 h apart and 24 h before tissues were harvested for analysis. FIG. 5 is based on Reyniers J P, et al., (1981), J. Biol. Chem. 256; 22, 11591-11594.

FIG. 6 details intraperitoneal administration of queuine to deficient humans. In FIG. 6, assuming a 32.5 g mouse (typical of adult CF1 mice) and a 70 kg human, the equivalent dose for a human would be 700 mg in each injection. FIG. 6 shows a dot plot of the predicted effect of intraperitoneally injected queuine on liver Q(+)tRNA concentration (e.g., histidyl tRNA, asparaginyl tRNA) in a moderately depleted 70 kg human.

FIG. 7 details oral administration of queuine to deficient animals; the indicated quantities of free queuine in FIG. 7 were fed to germ-free mice that had been completely depleted of (Q+)tRNA and the tissues were then harvested for analysis of hepatic Q(+)tRNAHis/Asp concentrations. FIG. 7 is based on Reyniers J P, et al., (1981), J. Biol. Chem. 256; 22, 11591-11594.

FIG. 8 details oral administration of queuine to deficient animals. Scaling the per-gram of body weight dosages yields a rough estimate of the results that would be expected from a short, high-dose regimen of oral supplemental queuine to restore proper BH4 redox cycling and associated neurotransmitter synthesis. FIG. 8 shows a dot plot of the predicted effect of orally administered queuine on liver Q(+)tRNA concentration (e.g., histidyl tRNA, asparaginyl tRNA) in a totally depleted 70 kg human.

### DETAILED DESCRIPTION

The gut microbiome of mammals is critical for many basic functions relating to gut health, including metabolizing fermentation substrates ingested or produced by the host to generate short chain fatty acids used by the host. Gut microbes are also capable of detoxifying undesired compounds, training the immune system, stimulating/regulating intestinal cell growth and development, inhibiting gut colonization by harmful bacteria, fungi and other pathogens, and producing certain vitamins for the host, such as biotin and vitamin K. Other important functions of gut microbes include production of hormones that mediate fat storage, modulating colonic pH, and regulating water and sodium absorption in the gut. Interactions between the gut and other organs in the body, including the liver, adipose tissue and brain, further explain the critical impacts of gut microbial ecology on host health.

Gut microbiome composition varies between individuals depending on such factors as diet, genetics, age and exposure to antibiotics (see e.g., Salonen, A. & de Vos, W. M. Impact of diet on human intestinal microbiota and health. Annu Rev Food Sci Technol 5, 239-262). Variations in gut microbiome constitution and health can allow pathogenic species to colonize, multiply and subsequently outnumber beneficial bacterial species. Causes of dysbiosis can range from changes or deficiencies in host diet, immune function, disease and other host-related factors, colonization of the gut by competing microbes (e.g., fungi) and other pathogens (that may toxify, deplete or otherwise adversely change the gut environment), adverse exposure of the gut to antibiotics, toxins and even “nutrients” that alter the gut environment or microbiome composition/balance, and other exogenous factors. The particular form and manifestation of dysbiosis can range from a global microbiome “crash” (e.g., attributable to heavy antibiotic use, or severe host disease), to a critical shift in population makeup that diminishes or eliminates key taxa, to more discrete impacts that selectively alter viability, reproduction, metabolism, or biosynthesis of critical products by heirloom taxa or other critical microbiome community members.

Among the best known, critical metabolic functions of gut microbes involves the production of short chain fatty acid butyrate by butyrogenic bacteria. Butyrate plays a regulatory role in transepithelial fluid transport in the gut, limits mucosal inflammation and oxidative status, reinforces the gut epithelial defense barrier, and helps regulate visceral sensitivity and intestinal motility. Recent studies implicate butyrate as a potential factor in prevention of colorectal cancer. Systemically, butyrate may also help limit metabolic diseases such as hypercholesterolemia, insulin resistance, and ischemic stroke. Only a limited number of anaerobic intestinal bacteria are known to produce butyrate, and these bacteria are notably depleted in the gastrointestinal (GI) tracts of patients with metabolic diseases.

An excess of pathogenic bacterial species in the gut is also associated with reduction of butyrogenic bacteria in the GI tract, as observed in association with several immune-related, inflammation-related and other disease conditions, including cancer (e.g., colorectal cancer), inflammatory bowel disease (IBD) (e.g., Crohn's disease, ulcerative colitis), irritable bowel syndrome (IBS), type 2 diabetes mellitus, obesity, bacterial or viral diarrhea, constipation, bloating, allergies, urinary tract infections, and others.

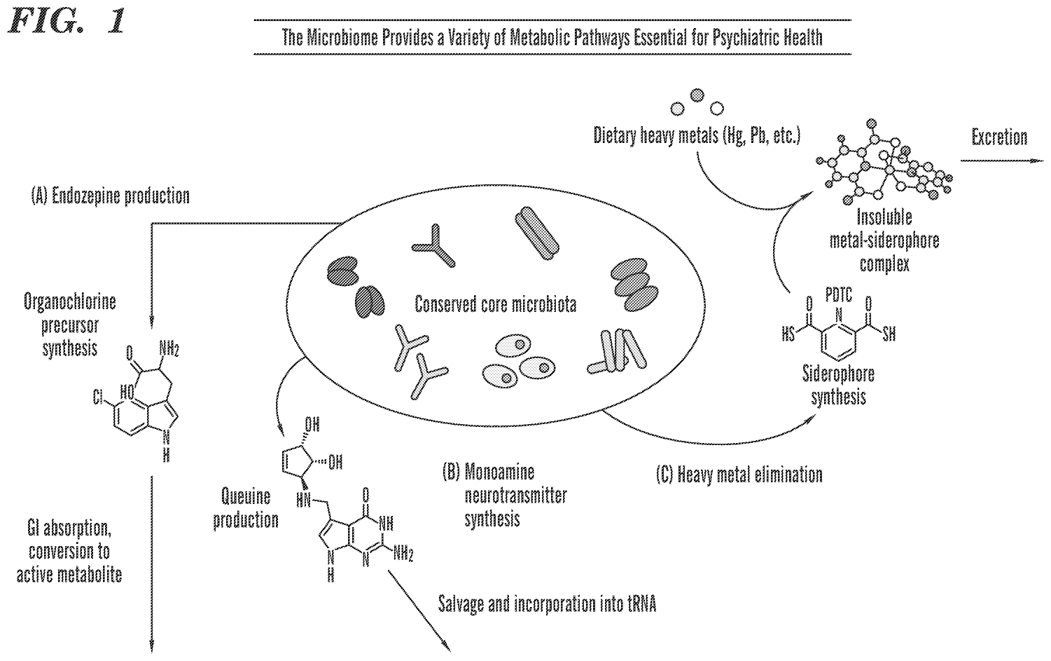

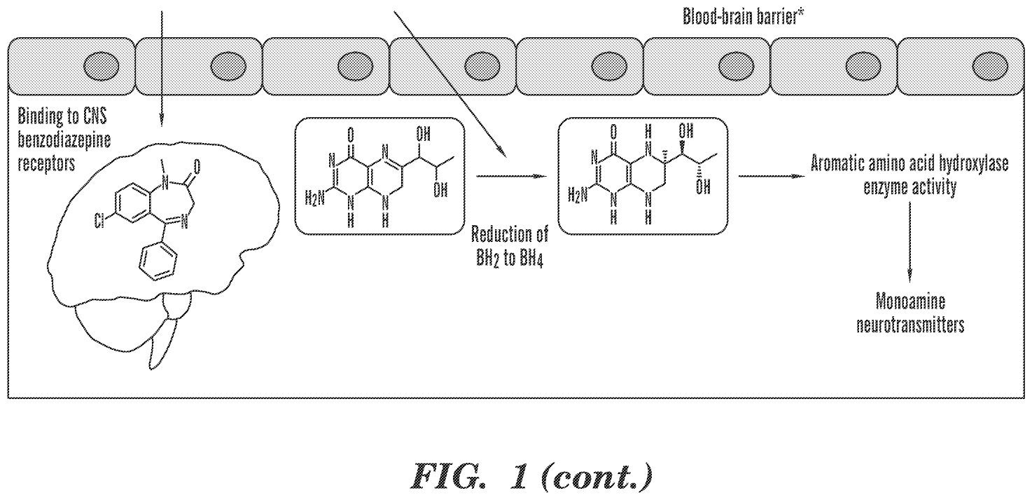

The technology described herein relates to more complex, and less well known, gut bacterial products and processes that impact central nervous system (CNS) functions of hosts. Within principal aspects of the technology described herein, selected heirloom bacterial species are identified that possess ex-genes directing metabolic pathways yielding important products utilized in development, maintenance and/or normal functioning of the mammalian CNS. The subject bacteria are isolated, prepared and optionally formulated for improved delivery or function a dysbiotic mammalian subject, to effectively treat or prevent one or more CNS conditions or other symptom(s) attributable to dysbiosis. The technology described herein focuses in principal aspects on clinical cases of gut dysbiosis associated with adverse impacts on CNS function. As illustrated in FIG. 1, the mammalian CNS is constructed in part and otherwise regulated by a diversity of metabolic pathways and products, many of which are targeted within the technology described herein as having their normal function influenced by, or even dependent on, a healthy and complete gut microbiome. The following describes various aspects of the technology and considerations to permit one of ordinary skill in the art to prepare and use the disclosed compositions and methods, e.g., to treat or prevent a CNS disease or disorder as described.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “administering,” refers to the placement of a compound as disclosed herein into a subject by a method or route which results in at least partial delivery of the agent at a desired site. Compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

Specifically, as used herein “administer” and “administration” encompasses embodiments in which one person directs another to consume live bacteria, dead bacteria, spent medium(s) derived from a bacteria, cell pellet(s) of a bacteria, purified metabolite(s) produced by bacteria, purified protein(s) produced by a bacteria, prebiotics, small molecules, queuine, queuine analogs, queuine precursors, prebiotics, endozepines, siderophores, or combinations thereof in a certain manner and/or for a certain purpose, and also situations in which a user uses any of the above in a certain manner and/or for a certain purpose independently of or in variance to any instructions received from a second person. Non-limiting examples of embodiments include the situation in which one person directs another to consume live bacteria, dead bacteria, spent medium(s) derived from a bacteria, cell pellet(s) of a bacteria, purified metabolite(s) produced by bacteria, purified protein(s) produced by a bacteria, prebiotics, small molecules, queuine, queuine analogs, precursors of queuine, endozepines, siderophores, or combinations thereof in a certain manner and/or for a certain purpose include when a physician prescribes a course of conduct and/or treatment to a patient, when a parent commands a minor user (such as a child) to consume such a product, when a trainer advises a user (such as an athlete) to follow a particular course of conduct and/or treatment, or when a manufacturer, distributer, or marketer recommends conditions of use to an end user, for example through advertisements or labeling on packaging or on other materials provided in association with the sale or marketing of a product. In some embodiments, the disclosed compositions can be administered orally, intravenously, intramuscularly, intrathecally, subcutaneously, sublingually, buccally, rectally, vaginally, by the ocular route, by the otic route, nasally, via inhalation, by nebulization, cutaneously, transdermally, or combinations thereof, and formulated for delivery with a pharmaceutically acceptable excipient, carrier or diluent. Of note, although the disclosed compositions encompass multiple formulations and modes of delivery for treatments to ameliorate dysbiosis and its sequelae, it should be noted that live biotherapeutic products such as probiotics are not typically administered intravenously, intramuscularly, or intraperitoneally. These modes of delivery would likely be reserved for small-molecule products of bacterial metabolism including but not limited to queuine or queuosine, endozepine precursors, heavy metal-chelating small molecules or peptides, or other such compounds as described herein.

As used herein, the term “isolated” encompasses a bacterium or other entity or substance that has been (1) separated from at least some of the components with which it was associated when initially produced (whether in nature, such as human stool, or in an experimental setting, such as a Petri plate consisting of artificial growth medium), and/or (2) produced, prepared, purified, and/or manufactured by the hand of man. Isolated bacteria, proteins, metabolites, or combinations thereof may be separated from at least about 10%, about 20%, about 30%, about 40%, about 50%, about 60%, about 70%, about 80%, about 90%, or more of the other components with which they were initially associated. In some embodiments, isolated bacteria, proteins, metabolites, or combinations thereof are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components (such as other bacterial species). The terms “purify,” “purifying” and “purified” refer to a bacterium or other material that has been separated from at least some of the components with which it was associated either when initially produced or generated (e.g., whether in nature or in an experimental setting), or during any time after its initial production, as recognized by those skilled in the art of bacterial cultivation or of relevant skill (e.g. chemistry). A bacterium or a bacterial population can be considered purified if it is isolated at or after production, such as from a material or environment containing the bacterium or bacterial population, and a purified bacterium or bacterial population can contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. In some embodiments, a bacterium or population of bacteria is “isolated” if it comprises a single strain of bacteria. In some embodiments, such isolated bacteria can be admixed or administered with other isolated bacteria, e.g., in a defined consortium of isolated bacteria. Bacterial compositions and the bacterial components thereof are generally purified from residual habitat products.

As used herein, “probiotic” is understood to mean “live microorganisms which when administered in adequate amounts confer a health benefit on the host”, as currently defined by the World Health Organization.

As used herein, “prebiotic” is understood to mean an ingredient that allows specific changes, both in the composition and/or activity in the gastrointestinal microbiota that may (or may not) confer benefits upon the host. Favored prebiotics will be those which encourage growth of probiotic compositions or their beneficial functions, but not growth of pathogens nor genes associated with pathogenicity (e.g. toxins).

As used herein, “medical food” is understood to mean “a food which is formulated to be consumed or administered enterally under the supervision of a physician and which is intended for the specific dietary management of a disease or condition for which distinctive nutritional requirements, based on recognized scientific principles, are established by medical evaluation”, as defined by 5(b) of the Orphan Drug Act (21 U.S.C. 360ee (b) (3)).

As used herein “initial amount” is understood to mean the amount of a substance, e.g., queuine, endozepines, siderophores, or levels of a given bacteria or function in an aliquot or sample prior to administration of the disclosed compositions to the subject. Initial amount can be measured in terms of concentration. As a non-limiting example, an initial amount can be measured in terms of nanograms of substance per milliliter of sample, e.g., nanograms of queuine per milliliter of blood or serum (ng queuine/mL blood or serum). The initial amount can also be measured, for instance, as the amount of queuine in regions of the brain, liver, whole or fractionated blood, or other relevant tissues prior to administration of the disclosed compositions. The amount of queuine can be represented in terms of millimoles of queuine per kg tissue. The initial amount can also be represented as a percentage of tRNAHis/Asp/Tyr/Asn which contains queuosine or a glycosylated queuosine derivative in the “wobble” position (position 34) of the anticodon, rather than guanosine. This percentage can depend on tissue type and tRNA type, and can be as low as 10-20% in skin, and as high as 100% in tissues such as brain. The initial amount can also be measured, for instance, as the amount of queuine or queuosine in a subject's stool sample prior to administration of queuine-producing bacteria to the subject. The amount of queuine can be represented in terms of nanograms of queuine per gram of stool (μg queuine/g stool). The initial amount can also be the level of expression of queuine producing enzymes or bacteria in the stool (log change of reads), as measured by qPCR, next-generation DNA or RNA sequencing, or other appropriate method. Unless otherwise defined herein, stool is weighed when wet or dry.

As used herein, a “host genetic response” means the response of a given organ and/or tissue (e.g., the brain, liver, or vagus nerve) on the gene expression level after exposure to disclosed compositions.

As used herein, “queuine producing bacteria” is understood to mean bacteria that can produce measurable quantities of queuine, as detected by LC/MS, ELISA, or other appropriate analytical assays. In some embodiments, queuine producing bacteria can produce queuine or its precursors under the physiological conditions in a human, e.g., under the pH, and temperature of the human gut. In some embodiments, queuine-producing bacteria express at least one gene involved in queuine biosynthesis once delivered to the human gastrointestinal tract.

As used herein, “human gut queuine producing bacteria” is understood to mean queuine producing bacteria that have been found in or isolated from the human gastrointestinal tract, or samples derived therefrom (e.g. fecal samples, colonic washes, or biopsies). In some embodiments, human gut queuine producing bacteria are identified by sequencing methods (e.g. qPCR or metagenomics) or by cultivation.

As used herein, “keystone human gut queuine producing bacteria” is understood to mean queuine producing bacteria that have been found or isolated from in the human gastrointestinal tract, or samples derived therefrom (e.g. fecal samples, colonic washes, or biopsies), that also express one or more genes involved in queuine biosynthesis in the mammalian gut or under physiologically relevant conditions. In some embodiments, keystone human gut queuine producing bacteria are identified by combining RNA and DNA based sequencing methods (e.g. transcriptomics, qPCR or metagenomics).

As used herein, “physiologically relevant conditions” of the human intestinal tract is understood to mean conditions that bacteria are exposed to in the human intestinal tract. In some embodiments this means a pH range that exists in the body. For instance, a pH range that is physiologically relevant to the human gut can be in the range of about 4.5 to about 7.5. In other embodiments it means exposure to other human gut bacteria, or carbon, nitrogen, nutrients or other compositions, e.g., mucin or phosphatidylcholine, in concentrations and combinations found in the intestinal tract.

As used herein, the term “gut” is understood to refer to the human gastrointestinal tract, also known as the alimentary canal. The gut includes the mouth, pharynx, oesophagus, stomach, small intestine (duodenum, jejunum, ileum), large intestines (cecum and colon) and rectum. While the entire alimentary canal can be colonized by varying species of microbes, the majority of the gut microbiome, in terms of both numbers of species of biomass, resides in the intestines (small and large).

As used herein, “bacteria” or “bacterial strain” is understood to mean a species or related taxonomic group of bacteria. A “bacterium” is understood as a single bacterial cell of a given species or related taxonomic group of bacteria.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a queuine, endozepine and/or heavy metal-related disease or disorder. The term “treating” includes reducing or alleviating at least one adverse effect or symptom of a condition, disease or disorder associated with a queuine, endozepine and/or heavy metal-related disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). A treatment need not cure a disorder (i.e., complete reversal or absence of disease) to be considered effective.

As used herein, the term “Uniprot ID” refers to an accession number, which when used as in input for the publicly available database Uniprot, permits access to information, such as nucleotide or amino acid sequence of a gene, and the bacterium or yeast encoding that sequence in its genome. For a given Uniprot ID, the relevant information can be accessed on the world wide web (see e.g., “uniprot.org/uniprot/XXXX”, where “XXXX” is the Uniprot ID). Additionally, SEQ ID NOs that are amino acid sequences (e.g., SEQ ID NOs: 3660-90760, 91407-95263, 95292-95321) list additional information in the sequence information, which corresponds to the following: database|Unique Identifier|Entry Name Protein Name OS=Organism Name OX=Organism Identifier GN=Gene Name PE=Protein Existence SV=Sequence Version.

As used herein, “dysbiosis” refers to any structural or functional imbalance or disruption within a normal, healthy gut microfloral community. This can include large or small changes in gut microbiome community composition, for example a decline in numbers of one or more key species (e.g., due to decreased viability and/or proliferative capacity), crashing of a wide diversity of species (e.g., due to radical changes in the gut environment, such as may be caused by host disease or antibiotic use) changes in relative abundance between multiple microbial species (e.g., outgrowth or inhibition by one or more competing fungal or bacterial species, depriving other community members of resources or otherwise impairing their growth, viability, and/or function (e.g., metabolic activity and output of specific products)), or any other circumstance that significantly alters the gut microfloral community structure, health, viability, stability, regenerative capacity, dominance/abundance of species, and/or specific biological and metabolic function of individual species and/or the community as a whole.

As used herein, “genes involved in queuine biosynthesis” and “queuine synthesis genes” refer to the genes themselves and/or the enzymes or proteins that are their gene products (denoted in parentheses), including, but not limited to: folE (GTP cyclohydrolase), QueD (6-carboxy-5,6,7,8-tetrahydrobiopterin synthase), QueE (7-carboxy-7-deazaguanine synthase), QueC (7-cyano-7-deazaguanine synthase, PreQ0 synthase), QueF (7-cyano-7-deazaguanine reductase, PreQ0 reductase), tgt or btgt (tRNA guanine transglycosylase, bacterial tRNA guanine transglycosylase), QueA (S-adenosylmethionine:tRNA ribosyltransferase-isomerase), QueG or QueH (both are epoxyqueuosine reductases), the PreQ1_1_Riboswitch, the PreQ1_2_Riboswitch, the PreQ1_3_Riboswitch, or combinations thereof.

As used herein, “queuine related metabolite” refers to any metabolite directly or indirectly influenced by queuine levels in the host. Non limiting examples include biopterins (particularly BH2 and BH4); monoamine neurotransmitters; imidazoleamines, e.g. histamine; catecholamines, e.g. adrenaline (epinephrine), dopamine, noradrenaline (norepinephrine); indolamines, e.g., serotonin (5-HT), melatonin; and other metabolites influenced by queuine, e.g. nitric oxide, phenylalanine, tyrosine, tryptophan, and kynurenine, or combinations thereof.

As used herein, “queuine analogs” refers to structural variants of queuine or a molecule or macromolecule comprising queuine or structural variants thereof that retains one or more activities of queuine (e.g., tRNA-queuosine translation, regeneration of BH4, synthesis of monoamine neurotransmitters, etc.). Non-limiting examples of queuine structural variants are described further herein. In some embodiments, the queuine analogs are in a form adapted for oral use or administration. In particular, a queuine analog can be part of a covalent or ionic complex, such as queuosine, a mannosyl queuosine, galactosyl queuosine, or a glutamyl queuosine. It can also be considered in the form of tRNA-queuosine or an oligonucleotide comprising queuosine. Among these derivatives are the glycosylated derivatives of queuine and queuosine, such as mannosylqueuine, galactosylqueuine, and aminoacylated derivatives such as glutamylqueuine.

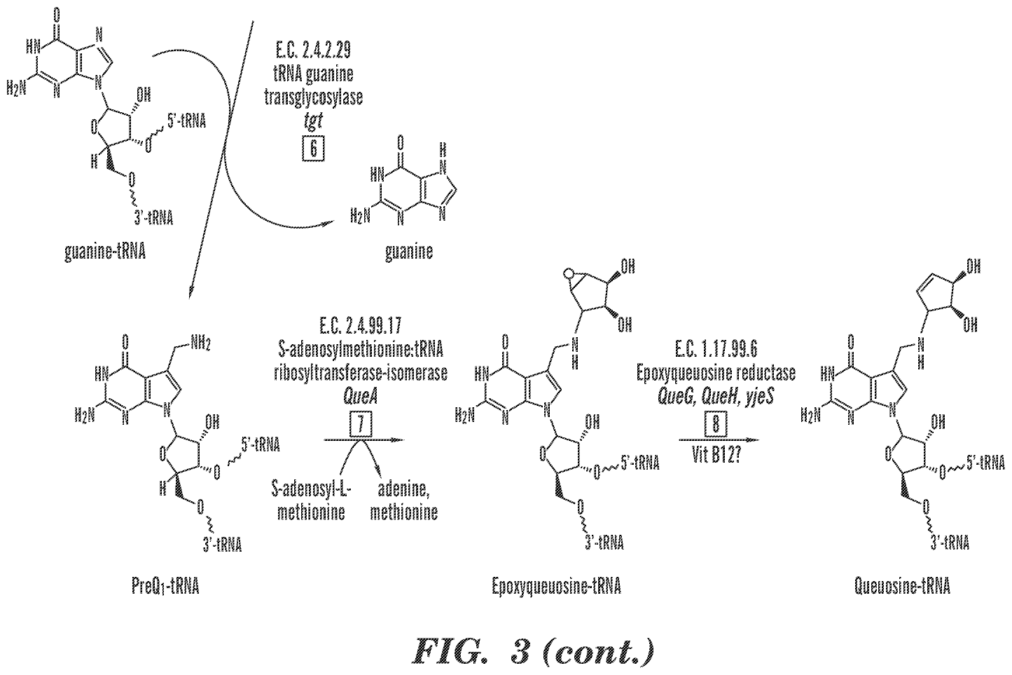

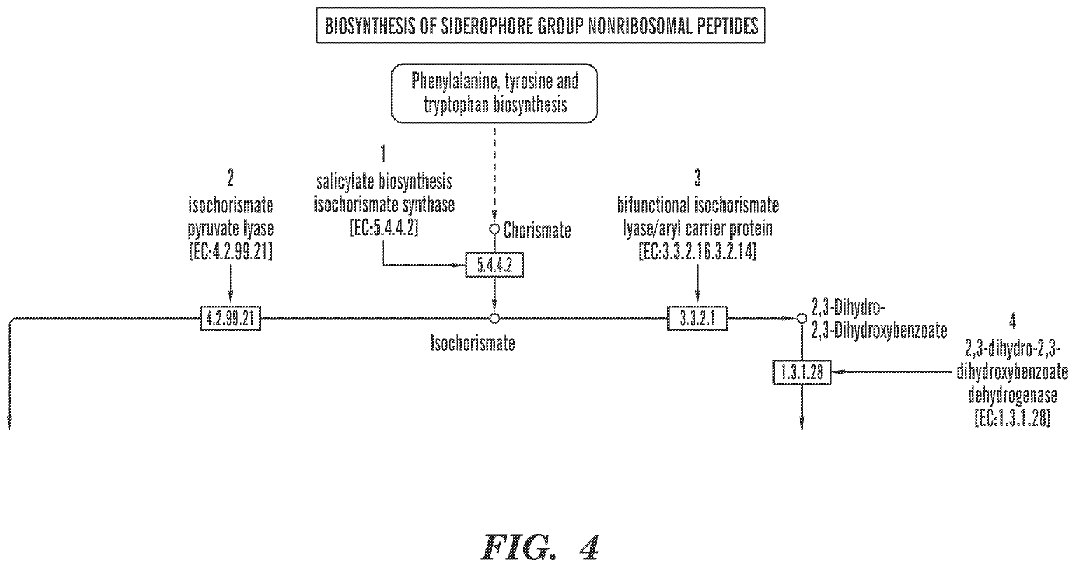

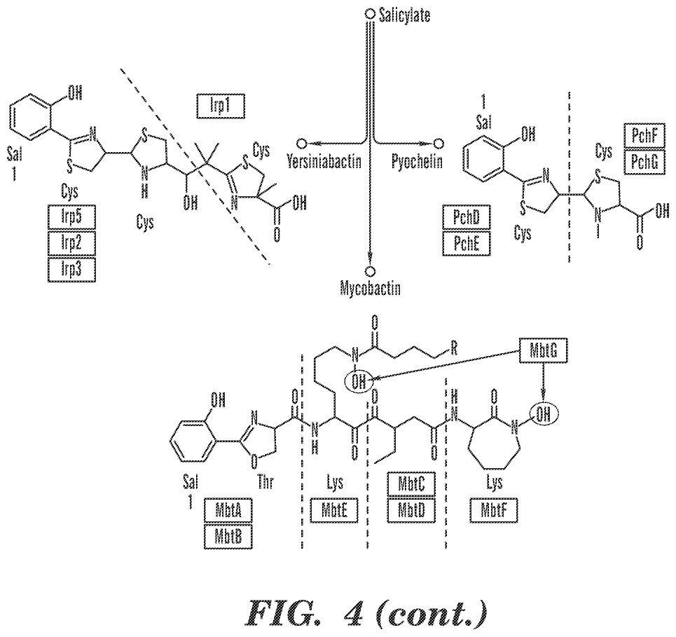

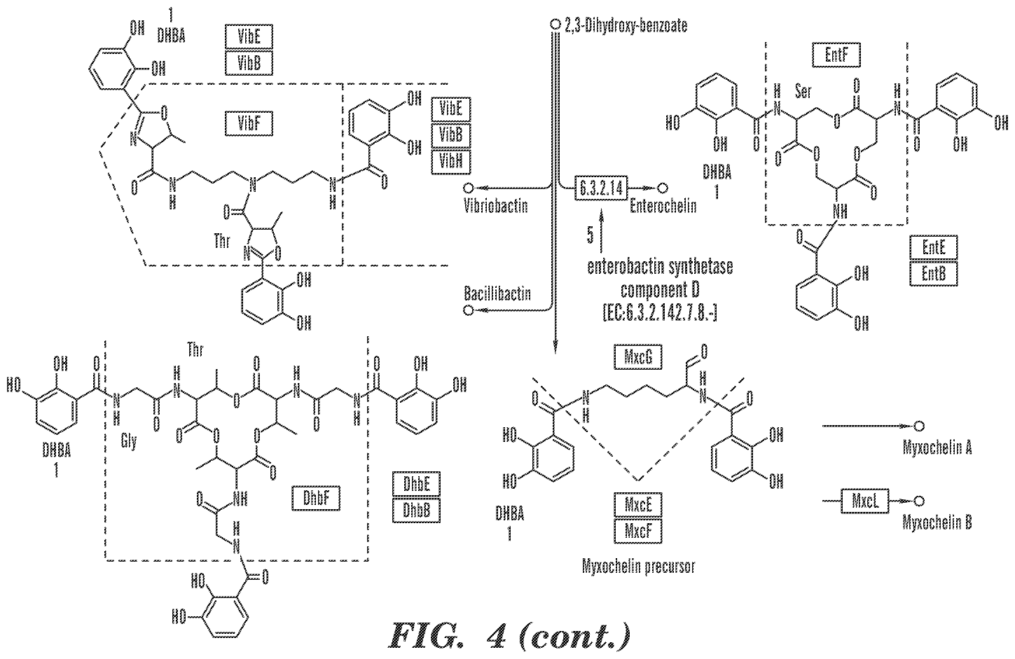

As used herein, “queuine precursor” refers to any molecule listed in FIG. 3. Non limiting examples of a queuine precursor are its intermediate precursor, epoxyqueuine, whether in free form or in the form of a covalent complex with molecules or macromolecules. In another non-limiting example, a queuine precursor refers to cofactors required for queuine biosynthesis in bacteria, such as Vitamin B-12, generally referred to as cobalamin, which is required for a functional QueG.

As used herein, “endozepine producing bacteria” refers to any bacterium with the capability to produce an endozepine or a precursor of an endozepine, which is then converted to an endozepine by the host or its native microbiome.

As used herein, “endozepine producing yeast” refers to any yeast with the capability to produce an endozepine or a precursor of an endozepine, which is then converted to an endozepine by the host or its native microbiome

As used herein, “heavy metal sequestering bacteria” refers to any bacterium that can produce a substance that binds or complexes with a heavy metal thereby reducing bioavailability of the heavy metal or a bacterium that can actively import toxic heavy metals such as mercury and lead. In particular, a heavy metal sequestering bacteria can do this through production of a siderophore with an affinity to mercury, lead, or another toxic heavy metal. As used herein, “siderophore” refers to small peptidic molecules, readily assembled by short, dedicated metabolic pathways, which contain side chains and functional groups that can provide a high-affinity set of ligands for coordination of metals. Alternatively, a heavy metal sequestering bacteria can do this through production of extracellular polymeric substances that bind to the heavy metals, or by actively or passively transporting the heavy metals and sequestering internally in a vesicle.

As used herein, the term “clinical improvement” encompasses improvement in a measure of disease or symptom severity. Such improvement can include an increase, as that term is used herein, in the level of queuine or a queuine metabolite, endozepine or an endozepine metabolite, or a decrease, as that term is used herein, in bioavailable heavy metal. Clinical improvement can also be indicated by a change for the better in a clinically-accepted rating or scale of a CNS disease or disorder, e.g., a change of at least one level in such a clinically-accepted rating or scale of a CNS disease or disorder. In a non-limiting example, clinical improvement would refer to a change for the better by at least one level or by at least 10% or greater improvement in: HAM-D (Hamilton depression rating scale) score of a patient with depression (e.g., after eight weeks of treatment with a composition as described herein), PANSS (Positive and Negative Syndrome Scale) of a patient with schizophrenia (e.g., after eight weeks of treatment with a composition as described herein), BAI (Beck Anxiety Index) of a patient with anxiety or related disorders (e.g., after eight weeks of treatment with the composition). In another non-limiting example, “clinical improvement” would refer to a 50% or greater reduction in the rate of progression of global cortical atrophy (Pasquier scale score) in a patient with a neurodegenerative disease, as measured by neuroimaging or similar techniques (e.g., within eight weeks of commencement of treatment with a composition as described herein). In another non-limiting example, “clinical improvement” would refer to a 50% or greater reduction in the rate of increase of UPDRS (Unified Parkinson's Disease Rating Scale) score or similar metric (e.g., within eight weeks of commencement of treatment with a composition as described herein). In another non-limiting example, “clinical improvement” would refer to a 25% or greater reduction in the severity of symptoms of autism spectrum disorder as measured by the CARS (Childhood Autism Rating Scale) assessment as administered by a qualified psychiatric professional (e.g. within twenty four weeks of commencement of treatment with a composition as described herein).

As used herein the terms “derivative” or “product derived therefrom” when used in reference to a bacterial or yeast strain refers to one or more modified live bacteria or yeast, dead bacteria or yeast, spent medium(s) derived from a bacteria or yeast, cell pellet(s) of a bacteria or yeast, purified metabolite(s) produced by bacteria or yeast, purified protein(s) produced by a bacteria or yeast, or combinations thereof.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a queuine, endozepine and/or heavy metal-related disease or disorder. A subject can be male or female.

A subject can be one who has been previously diagnosed with or identified as suffering from or having a condition in need of treatment (e.g. a queuine, endozepine and/or heavy metal-related disease or disorder) or one or more complications related to such a condition, and optionally, has already undergone treatment for a queuine, endozepine and/or heavy metal-related disease or disorder or the one or more complications related to a queuine, endozepine and/or heavy metal-related disease or disorder. Alternatively, a subject can also be one who has not been previously diagnosed as having a queuine, endozepine and/or heavy metal-related disease or disorder or one or more complications related to a queuine, endozepine and/or heavy metal-related disease or disorder. For example, a subject can be one who exhibits one or more risk factors for a queuine, endozepine and/or heavy metal-related disease or disorder or one or more complications related to a queuine, endozepine and/or heavy metal-related disease or disorder or a subject who does not exhibit risk factors.

A “subject in need” of treatment for a particular condition can be a subject having that condition, diagnosed as having that condition, or at risk of developing that condition.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as descried herein. Accordingly, the percentage of “sequence similarity” is the percentage of amino acids which is either identical or conservatively changed; e.g., “sequence similarity”=(% sequence identity)+(% conservative changes). The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, a nucleic acid as described herein can be detected using PCR. In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR or quantitative RT-PCR (QRT-PCR) or real-time PCR methods. Methods of RT-PCR and QRT-PCR are well known in the art.

In some embodiments of any of the aspects, the level of a nucleic acid described herein can be measured by a quantitative sequencing technology, e.g. a quantitative next-generation sequencing technology. In some embodiments of any of the aspects, the sequence of a nucleic acid described herein can be determined using a next-generation sequencing technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing (i.e., dideoxy chain termination), high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

In some embodiments, sequencing comprises 16S rRNA gene sequencing, which can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes with a next-generation sequencing technology as described herein (see also e.g., U.S. Pat. Nos. 5,654,418; 6,344,316; and 8,889,358; and US Patent Application Numbers US 2013/0157265 and US 2018/0195111, which are incorporated by reference in their entireties).

In some embodiments, sequencing comprises 18S rRNA gene sequencing, which can also be referred to as “18S ribosomal RNA sequencing”, “18S rDNA sequencing” or “18S rRNA sequencing”. 18S rRNA is the eukaryotic cytosolic homologue of 16S ribosomal RNA in prokaryotes and mitochondria. Sequencing of the 18S rRNA gene can be used for genetic studies as it is highly conserved between different eukaryotic species, but it is not present in bacteria and archaea species. In addition to highly conserved regions, the 18S rRNA gene also comprises nine hypervariable regions (V1-V9) that vary by species. 18S rRNA gene sequencing typically comprises using a plurality of universal primers that bind to conserved regions of the 18S rRNA gene (e.g., conserved among fungi-specific 18S rRNA), PCR amplifying the eukaryotic 18S rRNA gene regions (including hypervariable regions), and sequencing the amplified 18S rRNA genes with a next-generation sequencing technology as described herein. In some embodiments, human 18S rRNA sequences can be excluded from any analysis, or primers specific for fungal 18S rRNA can be used (see also e.g., Banos et al., BMC Microbiol. 2018 Nov. 20; 18(1):190; U.S. Pat. Nos. 6,180,339 and 9,434,986, which are incorporated by reference in their entireties).

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within in nature.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### Queuine

“Queuine” is a hypermodified nucleobase found in the first (or wobble) position of the anticodon of tRNAs specific for Asn, Asp, His, and Tyr, in most eukaryotes and prokaryotes. Queuine is also known as “Q base” or 2-amino-5-((((1S,4S,5R)-4,5-dihydroxy-2-cyclopenten-1-yl)amino)methyl)-1,7-dihydro-4H-pyrrolo(2,3-d)pyrimidin-4-one. Queuine has the chemical structure of formula (I) below.

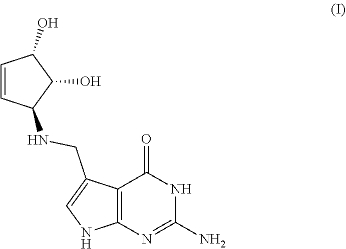 [SMILES: Nc1nc2[nH]cc(CN[C@H]3C=C[C@H](O)[C@@H]3O)c2c(=O)[nH]1]

Analogs and derivatives of queuine are discussed further herein in the section titled “Queuine Analogs, Queuine Precursors, and Queuine-Related Metabolites,” below.

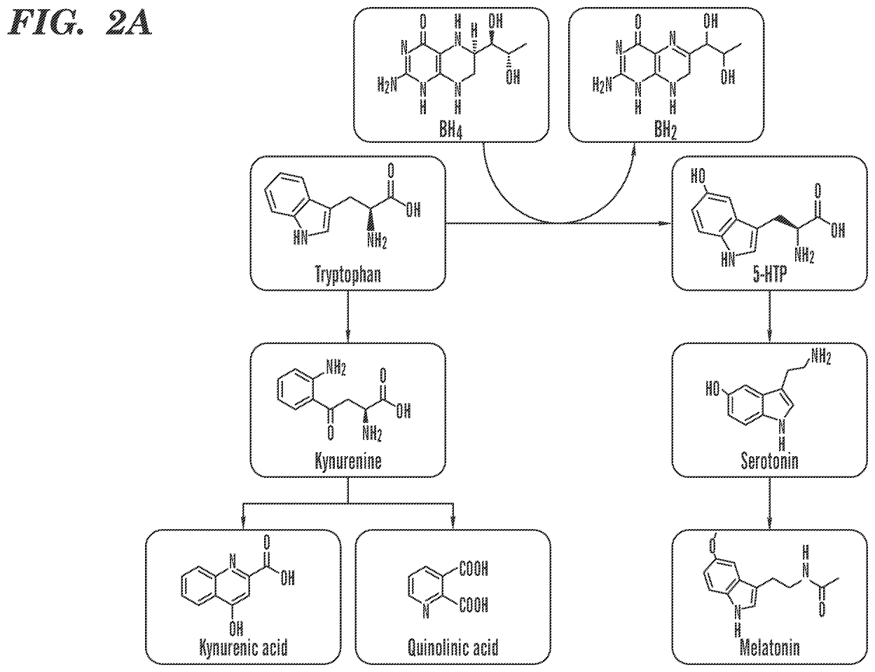

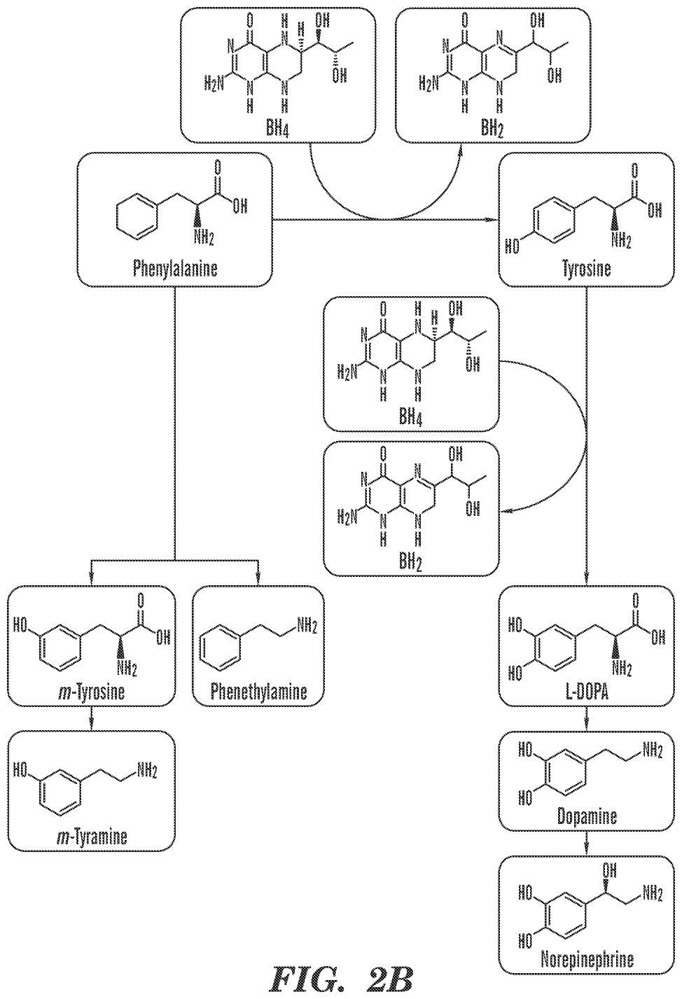

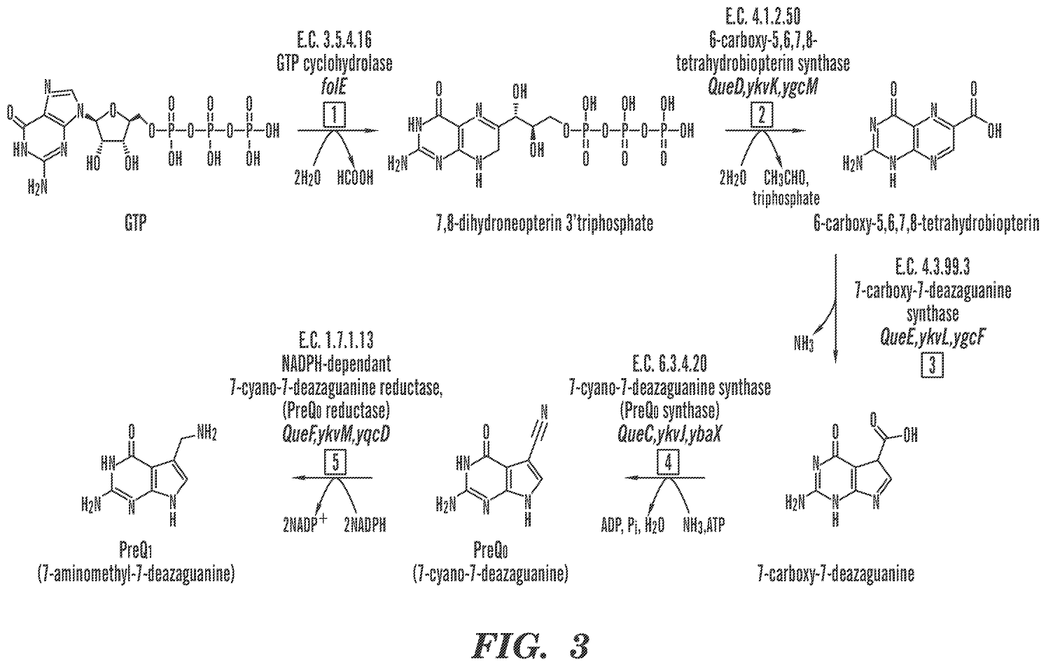

FIG. 1, Panel B illustrates an exemplary CNS metabolic pathway targeted within the technology described herein, defined herein as a “queuine-dependent monoamine neurotransmitter synthesis pathway”. Queuine is a modified nucleobase utilized by all eukaryotic organisms but produced exclusively by bacteria. While it is possible for queuine to be acquired through the diet, most, if not all, foods exhibit a low level of bioavailable queuine (see e.g., Example 1). Accordingly, bacteria in the gut microbiome produce the majority, if not all, of queuine that enters the bloodstream and crosses the blood brain barrier. Among the important activities described here for queuine, in regulating CNS function and mediating cognitive and mental health disorders in cases of queuine deficiency, queuine is involved in regenerating tetrahydrobiopterin (BH₄) from its oxidation product dihydrobiopterin (BH₂). BH₄ is essential for the synthesis of the monoamine neurotransmitters serotonin, norepinephrine, dopamine, melatonin, and nitric oxide (see e.g., FIG. 2A-2B).

Queuine is a modified nucleobase that richly illustrates the nature of symbiotic interdependence between microbes and their hosts. Queuine is synthesized exclusively by bacteria, but is utilized by nearly all eukaryotic organisms. Queuine promotes accurate translation of mRNA into peptides, enzymes, and proteins, ordinarily by hosts salvaging the compound from the GI tract and incorporating it as the nucleoside form (queuosine) into the anticodon of certain tRNAs (see e.g., Fergus, C., Barnes, D., Algasem, M. A. & Kelly, V. P. The queuine micronutrient: charting a course from microbe to man. Nutrients 7, 2897-2929).

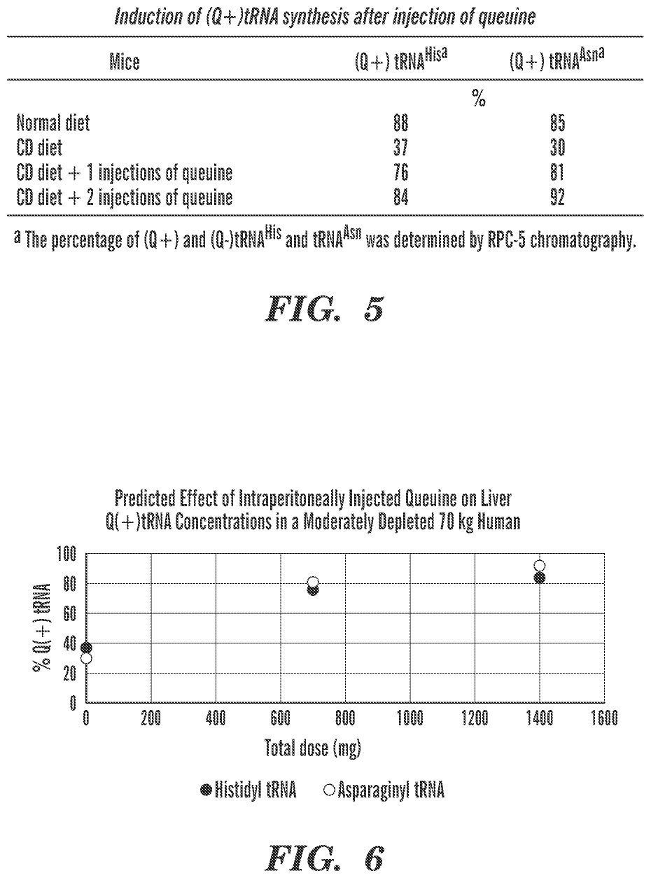

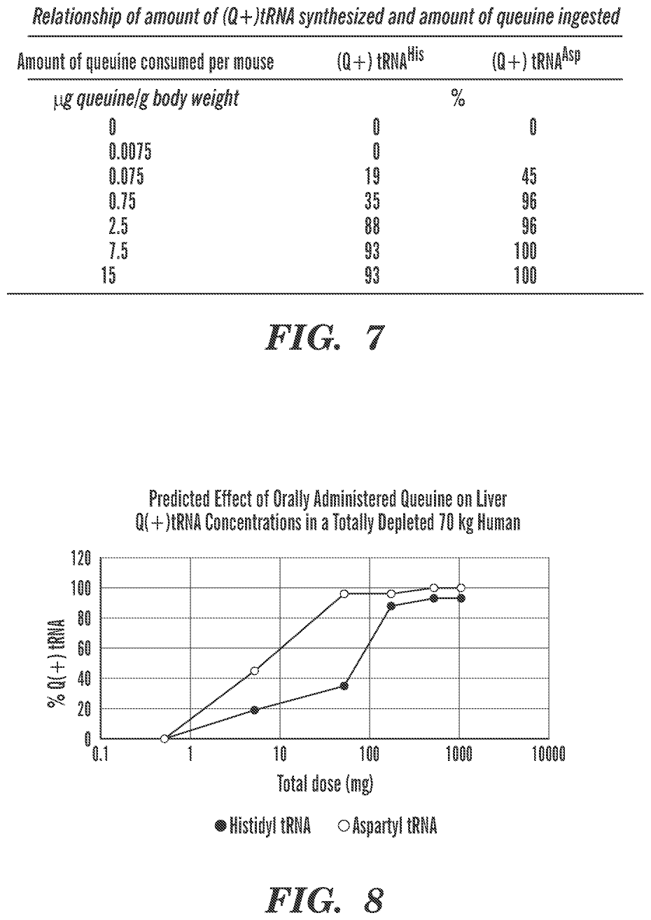

It has been demonstrated in mice that administration of exogenous queuine is essential for the biosynthesis of the queuosine-tRNAs (see e.g., Reyniers J P, et al., (1981), J. Biol. Chem. 256; 22, 11591-11594). Intraperitoneal administration of queuine to deficient mice resulted in a corresponding increase in (Q+)tRNA^{His} and (Q+)tRNA^{Asn} percentages of total (Q−)tRNA^{His} and (Q−)tRNA^{Asn}, respectively, in the liver (see e.g., FIG. 5). Similar results are predicted for humans (see e.g., FIG. 6). Oral administration of queuine produced a similar result in mice (see e.g., FIG. 7) and is also expected to produce a similar result in humans (see e.g., FIG. 8).

In bacteria, queuine conversion is affected by metabolic conversions of a 7-(aminomethyl)-7-deazaguanine, which is substituted for a guanine at position 34 (the “wobble” nucleotide) by a guanine transglycosylase. While this pathway is not known to occur in eukaryotes, the product of this pathway is critically significant in the metabolism of higher organisms.

Because queuine is essential for normal mammalian homeostasis but is not produced by the body, it is generally characterized as a “vitamin”. While queuine can be obtained from the diet, concentrations are generally small and highly variable across food sources. As a result, it has been previously undetermined whether the average diet can provide enough queuine to compensate for a deficiency of queuine production by the microbiome. To the extent that queuine is a nutrient sometimes classified as a vitamin, compositions as described herein that promote an increase in queuine production, e.g., in the gut, can be considered nutritional supplements.

The present disclosure provides for delivering a composition of one or more live bacteria, dead bacteria, spent medium(s) derived from a bacteria, cell pellet(s) of a bacteria, purified protein(s) produced by a bacteria, prebiotics, queuine, queuine analogs, queuine precursors, or combinations thereof, to increase queuine levels of the mammalian host. In some embodiments, a composition comprising a purified queuine-associated metabolite(s) produced by bacteria is administered to treat a queuine-associated disease or disorder as described herein; although such a metabolite does not necessarily increase the level of queuine, the metabolite can still be effective in treating a queuine-associated disease or disorder.

The present disclosure also provides methods for identifying mammalian subjects in need of the composition of one or more live bacteria, dead bacteria, spent medium(s) derived from bacteria, cell pellet(s) of bacteria, purified protein(s) produced by bacteria, prebiotics, queuine, queuine analogs, queuine precursors, or combinations thereof, to increase queuine levels of the mammalian host. In some embodiments, a method is described for identifying mammalian subjects in need of a composition comprising a purified queuine-associated metabolite(s) produced by bacteria to treat a queuine-associated disease or disorder as described herein

### Queuine-Producing Bacteria

In some embodiments the present disclosure provides one or more non-pathogenic queuine-producing bacterial strains (e.g., purified strains) and/or their derivatives (e.g. live bacteria, dead bacteria, spent medium(s) derived from a bacteria, cell pellet(s) of a bacteria, purified metabolite(s) produced by bacteria, purified protein(s) produced by a bacteria, or combinations thereof) and compositions comprising the same for administration to a subject in need thereof. The bacteria can be naturally occurring, or can be engineered (e.g., through strain engineering or selection) to produce queuine. In some embodiments, one strain of queuine-producing bacteria can be administered to a subject in need thereof. In some embodiments, multiple strains of queuine-producing bacteria can be administered to a subject in need thereof. In some embodiments, the one or more bacteria (e.g., purified bacteria) can act synergistically. For instance, the multiple bacteria can act synergistically to produce high levels of queuine, via, including but not limited to, cross feeding of nutrients or metabolites (including one or more queuine precursors) important for queuine production or via supporting growth or survival of queuine-producing bacteria. Accordingly, any one, or any combination of the queuine-producing bacteria described herein can be administered to a subject in need thereof. In one embodiment, a rationally designed consortium of bacteria can be assembled that in total encodes and expresses the enzymes sufficient to produce queuine.

In some embodiments, the bacteria described herein can produce queuine at or under physiologically relevant conditions, such as under the conditions of the human gut. In some embodiments, a pH relevant to the human gut is between about 4.5 and about 7.5. For instance, the pH can be about 4.5, 5.0, 5.5., 6.0, 6.5, 7.0 7.5, or any value between about 4.5 and 7.5. In some embodiments, the physiologically relevant conditions of the human gut include being exposed to carbon sources, nitrogen sources, or micronutrients found in the human gut (such as host-derived glycoproteins like mucin) or those in a typical human diet (e.g. complex or simple glycans).

In some embodiments, queuine producing bacteria are identified by the presence of genes involved in queuine biosynthesis (see e.g., FIG. 3), using genome sequencing, qPCR, or other related methods. In some embodiments, the queuine biosynthesis genes include but are not limited to folE, QueD, QueE, QueC, QueF, tgt, QueA, and QueG or QueH. In some embodiments, a queuine producing bacteria is classified as such by having 1, 2, 3, 4, 5, 6, 7, 8, or 9 of the genes involved in queuine biosynthesis (see e.g., FIG. 3, Table 1). In some embodiments, preference is given to bacteria that possess QueD, QueE, QueC, QueF, and tgt. In some embodiments, the genes encoding an enzyme involved in queuine biosynthesis are of at least 50% amino acid sequence similarity with the representative sequences SEQ ID NOs: 3660-82283 (e.g., at least 60% similarity, at least 70% similarity, at least 80% similarity, at least 90% similarity, at least 91% similarity, at least 92% similarity, at least 93% similarity, at least 94% similarity, at least 95% similarity, at least 96% similarity, at least 97% similarity, at least 98% similarity, at least 99% similarity, at least 99.5% similarity, at least 99.9% similarity, or 100% similarity). Enzymes produced by queuine biosynthesis genes from other species of bacteria will catalyze the same reactions as those of the reference or representative enzymes.

Table 1 highlights different categorizations of bacteria, based on the presence of genes involved in queuine biosynthesis and/or presence in the human gastrointestinal tract.

TABLE 1

| Gene | folE | QueD | QueE | QueC | QueF | tgt | QueA | QueG/H | Classification |
|---|---|---|---|---|---|---|---|---|---|
| Organism is assigned | + | + | + | + | + | + | + | + | Definite |
| (+) status for a given | | | | | | | | | Producer |
| gene if gene is present | +/− | + | + | + | + | + | +/− | +/− | Probable |
| in ≥50% of species | | | | | | | | | Producer |
| isolates. (−) otherwise. | +/− | + (any 3-4) | | | | + | +/− | +/− | Potential |
| | + (any 5 or more) | | | | | | | | Producer |
| (+/−) indicates that the | +/− | +/− | − | | | + | +/− | +/− | PreQ₁ |
| gene may be present or | | | | | | | | | Scavenger |
| absent in a microbe of | | | | | | | | | |
| that classification, as | | | | | | | | | |
| long as all other | | | | | | | | | |
| conditions of | | | | | | | | | |
| classification are met. | | | | | | | | | |

Table 2 lists Sequence IDs corresponding to bacteria 16S rRNA sequences identified to be queuine producing bacteria, human gut queuine producing bacteria, keystone human gut queuine producing bacteria, amino acid sequences for representative enzymes involved in the queuine biosynthesis pathway, bacterial genes involved in endozepine biosynthesis, 16S rRNA sequences of examples of endozepine producing bacteria or yeast, bacterial genes involved in siderophore biosynthesis, and 16S or 18S rRNA sequences of examples of endozepine producing bacteria or yeast.

TABLE 2
SEQ ID NO Key

| | | SEQ ID |
|---|---|---|
| Target | Description | NO |
| Queuine | Keystone human gut queuine producing bacteria (16S rRNA sequence) | 0001-0078 |
| | Human gut queuine producing bacteria (16S rRNA sequence) | 0001-0406 |
| | Queuine producing bacteria (16S rRNA sequence) | 0001-3659 |
| | GTP cyclohydrolase (folE; EC 3.5.4.16) | 3660- |
| | | 16762 |
| | 6-carboxy-5,6,7,8-tetrahydrobiopterin synthase (QueD; EC 4.1.2.50) | 16763- |
| | | 27170 |
| | 7-carboxy-7-deazaguanine synthase (QueE; EC 4.3.99.3) | 27171- |
| | | 35392 |
| | 7-cyano-7-deazaguanine synthase, PreQ0 synthase (QueC; EC 6.3.4.20) | 35393- |
| | | 43473 |
| | 7-cyano-7-deazaguanine reductase, PreQ0 reductase (QueF; EC 1.7.1.13) | 43474- |
| | | 50906 |
| | tRNA guanine transglycosylase, bacterial tRNA guanine transglycosylase | 50907- |
| | (TGT; EC 2.4.2.29) | 62499 |
| | S-adenosylmethionine:tRNA ribosyltransferase-isomerase (QueA; EC | 62500- |
| | 2.4.99.17) | 73907 |
| | epoxyqueuosine reductase (QueG or QueH; EC 1.17.99.6) | 73908- |
| | | 82283 |
| | PreQ1_1_Riboswitch | 90761- |
| | | 91292 |
| | PreQ1_2_Riboswitch | 91293- |
| | | 91361 |
| | PreQ1_3_Riboswitch | 91362- |
| | | 91398 |
| Endozepine | Endozepine producing bacteria or yeast (16S/18S rRNA sequence) | 91404- |
| | | 91406 |
| | Tryptophan_Halogenases | 82284- |
| | | 90702 |
| | Tomaymycin biosynthetic gene cluster | 90703- |
| | | 90719 |
| | Viridicatin biosynthetic gene cluster | 90720- |
| | | 90734 |
| | Sibiromycin biosynthetic gene cluster | 90735- |
| | | 90760 |
| | Pyrroloquinoline precursor producing bacteria (16S rRNA sequence) | 95264- |
| | | 95291 |
| | Pyrroloquinoline precursor gene | 95292- |
| | | 95321 |
| Heavy | Siderophore producing bacteria (16S rRNA sequence) | 91399- |
| Metals | | 91403 |
| | 2,3-dihydro-2,3-dihydroxybenzoate dehydrogenase (entA; EC: 1.3.1.28) | 91407- |
| | | 92334 |
| | isochorismatase (entB; EC: 3.3.2.1) | 92335- |
| | | 93030 |
| | enterobactin synthetase component D (entD; EC: 6.3.2.14) | 93031- |
| | | 93128 |
| | salicylate biosynthesis/isochorismate synthase (pchA; EC 5.4.4.2) | 93129- |
| | | 95220 |
| | Isochorismate pyruvate lyase (pchB; EC: 4.2.99.21) | 95221- |
| | | 95263 |

In some embodiments, the queuine producing bacteria can be identified by having a 16S nucleic acid sequence with a substantial percent identity to the 16S sequences of SEQ ID NOs: 0001-3659, which have been found to possess queuine producing genes encoded in their genomes. In some embodiments, the queuine-producing bacteria can have at least 90% 16S sequence identity to a 16S sequence given in Table 2 (e.g., at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity).

In some embodiments, the queuine-producing bacteria have been exemplified to include members of the human gut microbiome (henceforth known as “human gut queuine producing bacteria”). These bacteria include bacteria identified to be found in human fecal samples and/or cecal samples by metagenomics or cultivation based methods. In some embodiments, the human gut queuine producing bacteria are non-pathogenic bacteria belonging to a genus selected from the group consisting of: Acetobacter, Achromobacter, Acidaminococcus, Acinetobacter, Adlercreutzia, Aeribacillus, Aeromonas, Aggregatibacter, Akkermansia, Alistipes, Allisonella, Anaeroglobus, Anaerostipes, Aneurinibacillus, Anoxybacillus, Asaccharobacter, Bacillus, Bacteroides, Barnesiella, Bilophila, Blautia, Bordetella, Brenneria, Brevibacillus, Brevundimonas, Butyricimonas, Campylobacter, Cedecea, Chromohalobacter, Citrobacter, Clostridium, Coprococcus, Cronobacter, Delftia, Desulfovibrio, Dialister, Enterobacter, Enterorhabdus, Erysipelatoclostridium, Escherichia, Geobacillus, Haemophilus, Hafnia, Halobacillus, Helicobacter, Intestinibacter, Klebsiella, Kluyvera, Kosakonia, Kushneria, Lachnospira, Lachnotalea, Lactobacillus, Leclercia, Lelliottia, Litorilituus, Lysinibacillus, Lysobacter, Megasphaera, Micrococcus, Mitsuokella, Moellerella, Monoglobus, Moraxella, Morganella, Neisseria, Nosocomiicoccus, Noviherbaspirillum, Oceanobacillus, Ochrobactrum, Odoribacter, Oxalobacter, Paenibacillus, Pantoea, Parabacteroides, Paraprevotella, Parasutterella, Peptoniphilus, Planococcus, Porphyromonas, Prevotella, Proteus, Providencia, Pseudomonas, Psychrobacter, Raoultella, Roseomonas, Ruminococcus, Salinisphaera, Salinivibrio, Salmonella, Selenomonas, Senegalimassilia, Serratia, Shewanella, Shigella, Sphingomonas, Staphylococcus, Stenotrophomonas, Streptococcus, Succiniclasticum, Terribacillus, Thermotalea, Turicibacter, Veillonella, Vibrio, Victivallis, Virgibacillus, and Yersinia.

In some embodiments, the human gut queuine producing bacteria are non-pathogenic bacteria belonging to a species selected from the group consisting of: Acetobacter pasteurianus, Achromobacter xylosoxidans, Acidaminococcus fermentans, Acidaminococcus intestini, Acinetobacter baumannii, Acinetobacter calcoaceticus, Acinetobacter junii, Acinetobacter lwoffii, Acinetobacter pittii, Acinetobacter radioresistens, Acinetobacter schindleri, Acinetobacter towneri, Acinetobacter ursingii, Acinetobacter variabilis, Adlercreutzia equolifaciens, Aeribacillus pallidus, Aeromonas caviae, Aeromonas enteropelogenes, Aeromonas hydrophila, Aeromonas jandaei, Aeromonas salmonicida, Aeromonas schubertii, Aeromonas veronii, Aggregatibacter aphrophilus, Akkermansia muciniphila, Alistipes onderdonkii, Alistipes putredinis, Allisonella histaminiformans, Anaeroglobus geminatus, Anaerostipes caccae, Anaerostipes hadrus, Aneurinibacillus aneurinilyticus, Aneurinibacillus migulanus, Anoxybacillus flavithermus, Asaccharobacter celatus, Bacillus altitudinis, Bacillus amyloliquefaciens, Bacillus aquimaris, Bacillus atrophaeus, Bacillus badius, Bacillus bataviensis, Bacillus cereus, Bacillus circulans, Bacillus clausii, Bacillus coagulans, Bacillus cohnii, Bacillus endophyticus, Bacillus firmus, Bacillus flexus, Bacillus fordii, Bacillus galactosidilyticus, Bacillus halodurans, Bacillus infantis, Bacillus koreensis, Bacillus kyonggiensis, Bacillus lentus, Bacillus licheniformis, Bacillus litoralis, Bacillus marisflavi, Bacillus megaterium, Bacillus mojavensis, Bacillus mycoides, Bacillus nealsonii, Bacillus okuhidensis, Bacillus pseudofirmus, Bacillus pseudomycoides, Bacillus pumilus, Bacillus simplex, Bacillus sonorensis, Bacillus subterraneus, Bacillus subtilis, Bacillus thuringiensis, Bacillus timonensis, Bacillus vallismortis, Bacillus vietnamensis, Bacillus weihenstephanensis, Bacteroides caccae, Bacteroides cellulosilyticus, Bacteroides clarus, Bacteroides coprocola, Bacteroides dorei, Bacteroides eggerthii, Bacteroides faecis, Bacteroides fragilis, Bacteroides intestinalis, Bacteroides massiliensis, Bacteroides nordii, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bacteroides xylanolyticus, Barnesiella intestinihominis, Barnesiella viscericola, Bilophila wadsworthia, Blautia luti, Bordetella bronchiseptica, Bordetella trematum, Brenneria alni, Brevibacillus agri, Brevibacillus brevis, Brevibacillus choshinensis, Brevibacillus formosus, Brevibacillus laterosporus, Brevibacillus parabrevis, Brevundimonas diminuta, Butyricimonas virosa, Campylobacter coli, Campylobacter concisus, Campylobacter curvus, Campylobacter gracilis, Campylobacter jejuni, Campylobacter showae, Campylobacter ureolyticus, Cedecea lapagei, Cedecea neteri, Chromohalobacter japonicus, Citrobacter amalonaticus, Citrobacter braakii, Citrobacter farmeri, Citrobacter freundii, Citrobacter gillenii, Citrobacter koseri, Citrobacter murliniae, Citrobacter youngae, Clostridium acetireducens, Clostridium bartlettii, Clostridium beijerinckii, Clostridium botulinum, Clostridium butyricum, Clostridium carboxidivorans, Clostridium colicanis, Clostridium diolis, Clostridium disporicum, Clostridium novyi, Clostridium ramosum, Clostridium sporogenes, Clostridium thermocellum, Coprococcus catus, Coprococcus eutactus, Cronobacter sakazakii, Delftia tsuruhatensis, Desulfovibrio desulfuricans, Desulfovibrio fairfieldensis, Desulfovibrio piger, Dialister invisus, Dialister pneumosintes, Enterobacter aerogenes, Enterobacter asburiae, Enterobacter cloacae, Enterobacter hormaechei, Enterobacter kobei, Enterobacter ludwigii, Enterorhabdus caecimuris, Erysipelatoclostridium ramosum, Escherichia coli, Escherichiafergusonii, Escherichia hermannii, Escherichia marmotae, Geobacillus stearothermophilus, Haemophilus influenzae, Haemophilus pittmaniae, Hafnia alvei, Halobacillus dabanensis, Halobacillus karajensis, Halobacillus salinus, Halobacillus trueperi, Helicobacter pylori, Intestinibacter bartlettii, Klebsiella oxytoca, Klebsiella pneumoniae, Klebsiella variicola, Kluyvera cryocrescens, Kluyvera georgiana, Kosakonia cowanii, Kushneria sinocarnis, Lachnospira pectinoschiza, Lachnotalea glycerini, Lactobacillus mali, Leclercia adecarboxylata, Lelliottia amnigena, Litorilituus sediminis, Lysinibacillus boronitolerans, Lysinibacillus fusiformis, Lysinibacillus massiliensis, Lysinibacillus sphaericus, Lysinibacillus xylanilyticus, Lysobacter soli, Megasphaera elsdenii, Megasphaera micronuciformis, Micrococcus lylae, Mitsuokella jalaludinii, Moellerella wisconsensis, Monoglobus pectinilyticus, Moraxella osloensis, Morganella morganii, Neisseria canis, Neisseria cinerea, Neisseria elongata, Neisseria flavescens, Neisseria gonorrhoeae, Neisseria macacae, Neisseria meningitidis, Neisseria mucosa, Neisseria perflava, Neisseria subflava, Nosocomiicoccus massiliensis, Noviherbaspirillum denitrificans, Oceanobacillus iheyensis, Oceanobacillus oncorhynchi, Oceanobacillus sojae, Ochrobactrum anthropi, Odoribacter splanchnicus, Oxalobacter formigenes, Paenibacillus alvei, Paenibacillus amylolyticus, Paenibacillus barcinonensis, Paenibacillus barengoltzii, Paenibacillus daejeonensis, Paenibacillus dendritiformis, Paenibacillus glucanolyticus, Paenibacillus illinoisensis, Paenibacillus lactis, Paenibacillus larvae, Paenibacillus lautus, Paenibacillus macerans, Paenibacillus naphthalenovorans, Paenibacillus odorifer, Paenibacillus pabuli, Paenibacillus pasadenensis, Paenibacillus polymyxa, Paenibacillus rhizosphaerae, Paenibacillus stellifer, Paenibacillus thiaminolyticus, Paenibacillus typhae, Pantoea agglomerans, Parabacteroides distasonis, Parabacteroides goldsteinii, Parabacteroides gordonii, Parabacteroides johnsonii, Parabacteroides merdae, Paraprevotella clara, Parasutterella excrementihominis, Peptoniphilus asaccharolyticus, Peptoniphilus indolicus, Planococcus rifetoensis, Porphyromonas asaccharolytica, Porphyromonas bennonis, Porphyromonas somerae, Prevotella bivia, Prevotella buccae, Prevotella buccalis, Prevotella copri, Prevotella timonensis, Proteus mirabilis, Proteus penneri, Proteus vulgaris, Providencia alcalifaciens, Providencia heimbachae, Providencia rettgeri, Providencia stuartii, Pseudomonas aeruginosa, Pseudomonas alcaligenes, Pseudomonas bauzanensis, Pseudomonas caricapapayae, Pseudomonas chlororaphis, Pseudomonas fluorescens, Pseudomonas fragi, Pseudomonas fulva, Pseudomonas gessardii, Pseudomonas japonica, Pseudomonas libanensis, Pseudomonas lundensis, Pseudomonas luteola, Pseudomonas migulae, Pseudomonas monteilii, Pseudomonas mosselii, Pseudomonas oleovorans, Pseudomonas oryzihabitans, Pseudomonas putida, Pseudomonas rhodesiae, Pseudomonas saudiphocaensis, Pseudomonas stutzeri, Pseudomonas taetrolens, Pseudomonas tolaasii, Pseudomonas xanthomarina, Psychrobacter phenylpyruvicus, Raoultella ornithinolytica, Raoultella planticola, Roseomonas gilardii, Roseomonas mucosa, Ruminococcus albus, Ruminococcus callidus, Ruminococcus flavefaciens, Ruminococcus lactaris, Ruminococcus torques, Salinisphaera halophila, Salinivibrio costicola, Salmonella enterica, Salmonella enteritidis, Salmonella typhi, Selenomonas ruminantium, Selenomonas sputigena, Senegalimassilia anaerobia, Serratia marcescens, Serratia ureilytica, Shewanella xiamenensis, Shigella boydii, Shigella dysenteriae, Shigella flexneri, Shigella sonnei, Sphingomonas aerolata, Staphylococcus arlettae, Staphylococcus aureus, Staphylococcus auricularis, Staphylococcus capitis, Staphylococcus caprae, Staphylococcus carnosus, Staphylococcus cohnii, Staphylococcus condimenti, Staphylococcus devriesei, Staphylococcus epidermidis, Staphylococcus equorum, Staphylococcus gallinarum, Staphylococcus haemolyticus, Staphylococcus hominis, Staphylococcus hyicus, Staphylococcus intermedius, Staphylococcus kloosii, Staphylococcus lentus, Staphylococcus lugdunensis, Staphylococcus nepalensis, Staphylococcus pasteuri, Staphylococcus petrasii, Staphylococcus pettenkoferi, Staphylococcus saccharolyticus, Staphylococcus saprophyticus, Staphylococcus schleiferi, Staphylococcus sciuri, Staphylococcus simiae, Staphylococcus simulans, Staphylococcus succinus, Staphylococcus vitulinus, Staphylococcus warneri, Staphylococcus xylosus, Stenotrophomonas acidaminiphila, Stenotrophomonas maltophilia, Stenotrophomonas rhizophila, Streptococcus australis, Streptococcus bovis, Streptococcus equinus, Streptococcus gallolyticus, Streptococcus infantarius, Streptococcus infantis, Streptococcus lutetiensis, Streptococcus mitis, Streptococcus mutans, Streptococcus oralis, Streptococcus peroris, Streptococcus pseudopneumoniae, Streptococcus salivarius, Streptococcus sobrinus, Streptococcus thermophilus, Streptococcus tigurinus, Streptococcus vestibularis, Succiniclasticum ruminis, Terribacillus aidingensis, Terribacillus halophilus, Thermotalea metallivorans, Turicibacter sanguinis, Veillonella atypica, Veillonella denticariosi, Veillonella dispar, Veillonella parvula, Vibrio cholerae, Victivallis vadensis, Virgibacillus massiliensis, Yersinia bercovieri, Yersinia enterocolitica, Yersinia intermedia, Yersinia kristensenii, and Yersinia mollaretii.

In some embodiments, the human gut queuine producing bacteria can be identified as having a 16S nucleic acid sequence with a substantial percent identity to the 16S sequences of SEQ ID NOs: 0001-0406. In some embodiments, the human gut queuine-producing bacteria can have at least 90% 16S sequence identity to any of the 16S sequences listed in SEQ ID NOs 0001-0406 (e.g., at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity).

In some embodiments, the human gut queuine producing bacteria have been exemplified or determined to express genes involved in queuine biosynthesis in humans (henceforth known as “keystone human gut queuine producing bacteria”). In some embodiments, these keystone human gut queuine producing bacteria are non-pathogenic bacteria belonging to a genus selected from the group consisting of: Acidaminococcus, Adlercreutzia, Akkermansia, Alloprevotella, Anaerostipes, Arcobacter, Bacteroides, Barnesiella, Bilophila, Butyrivibrio, Campylobacter, Citrobacter, Clostridium, Coprobacter, Coprococcus, Desulfovibrio, Dialister, Dysgonomonas, Enterobacter, Enterorhabdus, Escherichia, Eubacterium, Fusobacterium, Haemophilus, Hafnia, Klebsiella, Megamonas, Megasphaera, Mitsuokella, Odoribacter, Oxalobacter, Parabacteroides, Porphyromonas, Proteus, Ruminococcus, Shigella, Streptococcus, Turicibacter, and Veillonella.

In some embodiments, the keystone human gut queuine producing bacteria are non-pathogenic bacteria belonging to a species selected from the group consisting of: Acidaminococcus fermentans, Adlercreutzia equolifaciens, Akkermansia muciniphila, Alloprevotella tannerae, Anaerostipes caccae, Anaerostipes hadrus, Arcobacter butzleri, Bacteroides caccae, Bacteroides cellulosilyticus, Bacteroides clarus, Bacteroides coprophilus, Bacteroides dorei, Bacteroides eggerthii, Bacteroides faecis, Bacteroides fragilis, Bacteroides massiliensis, Bacteroides nordii, Bacteroides oleiciplenus, Bacteroides ovatus, Bacteroides plebeius, Bacteroides salanitronis, Bacteroides salyersiae, Bacteroides stercoris, Bacteroides thetaiotaomicron, Bacteroides uniformis, Bacteroides vulgatus, Bacteroides xylanisolvens, Barnesiella intestinihominis, Bilophila wadsworthia, Butyrivibrio crossotus, Campylobacter curvus, Citrobacter freundii, Citrobacter koseri, Clostridium bartelettii, Clostridium ramosum, Coprobacter fastidiosus, Coprococcus catus, Coprococcus eutactus, Desulfovibrio piger, Dialister invisus, Dialister succinatiphilus, Enterobacter aerogenes, Enterobacter cancerogenus, Enterobacter cloacae, Enterorhabdus caecimuris, Escherichia coli, Eubacterium hallii, Fusobacterium mortiferum, Haemophilus pittmaniae, Haemophilus sputorum, Hafnia alvei, Klebsiella oxytoca, Klebsiella pneumoniae, Klebsiella variicola, Megamonas funiformis, Megamonas rupellensis, Megasphaera elsdenii, Megasphaera micronuciformis, Mitsuokella multacida, Odoribacter laneus, Odoribacter splanchnicus, Oxalobacter formigenes, Parabacteroides distasonis, Porphyromonas asaccharolytica, Porphyromonas uenonis, Ruminococcus callidus, Ruminococcus torques, Shigella sonnei, Streptococcus infantis, Streptococcus mitis, Streptococcus oralis, Streptococcus pneumoniae, Streptococcus tigurinus, Turicibacter sanguinis, Veillonella atypica, Veillonella dispar, Veillonella parvula, Dysgonomonas mossii, Proteus mirabilis, and Veillonella ratti.

In some embodiments, the keystone human gut queuine producing bacteria can be identified as having a 16S nucleic acid sequence with a substantial percent identity to the 16S sequences of SEQ ID NOs 0001-0078. In some embodiments, the human gut queuine-producing bacteria can have at least 90% 16S sequence identity to any of the 16S sequences listed in SEQ ID NOs 0001-0078 (e.g., at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity).

In some embodiments, additional keystone queuine producing bacteria can be identified by surveying human gastrointestinal samples (e.g. fecal samples, tissue biopsies, colonic washes) for RNA encoding queuine biosynthesis genes, and then identifying the bacteria expressing those transcripts. For example, one skilled in the art can leverage human fecal transcriptome sequencing to identify bacterial RNA sequences encoding queuine producing genes that are expressed in humans. These RNA sequences can then be mapped to public or private reference bacterial genomes (see e.g. the Human Microbiome Project website—available on the world wide web at hmpdacc.org/reference_genomes/reference_genomes.php) or genomes assembled de novo from the same samples (e.g., using assembly/annotation tools like Athena, MEGAHIT, Minia (available on the world wide web at github.com/GATB/minia), SPADES), to identify bacteria that express queuine producing genes in a human (“keystone human gut queuine producing bacteria”). See e.g., Bishara, A. et al. High-quality genome sequences of uncultured microbes by assembly of read clouds. Nat Biotechnol, (2018); Li et al. MEGAHIT: an ultra-fast single-node solution for large and complex metagenomics assembly via succinct de Bruijn graph. Bioinformatics 31, 1674-1676, (2015); Bankevich, A. et al. SPAdes: a new genome assembly algorithm and its applications to single-cell sequencing. Journal of computational biology: a journal of computational molecular cell biology 19, 455-477, (2012).

In some embodiments, the human gut queuine producing bacteria are non-pathogenic bacteria belonging to the genus Blautia, Coprococcus, or Dialister. In some embodiments, the human gut queuine producing bacteria are non-pathogenic bacteria belonging to species selected from the group consisting of: Blautia luti, Coprococcus catus, Coprococcus eutactus, Dialister invisus, or Dialister succinatiphilus. In some embodiments, a composition as described herein comprises Blautia luti (e.g., SEQ ID NO: 154). In some embodiments, a composition as described herein comprises Coprococcus catus (e.g., SEQ ID NO: 37). In some embodiments, a composition as described herein comprises Coprococcus eutactus (e.g., SEQ ID NO: 38). In some embodiments, a composition as described herein comprises Dialister invisus (e.g., SEQ ID NO: 40). In some embodiments, a composition as described herein comprises Dialister succinatiphilus (e.g., SEQ ID NO: 41).

In some embodiments, the keystone human gut queuine producing bacteria can be identified as having a 16S nucleic acid sequence with a substantial percent identity to the 16S sequences of SEQ ID NOs 37, 38, 40, 41, or 154. In some embodiments, the human gut queuine-producing bacteria can have at least 90% 16S sequence identity to any of the 16S sequences listed in SEQ ID NOs 37, 38, 40, 41, or 154 (e.g., at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity).

In some embodiments, the queuine producing bacteria are engineered to produce queuine constitutively or inducibly on its chromosome (one or multiple sites), a plasmid (high or low copy number), or both. A variety of different host bacteria can be engineered to produce queuine. For instance, in some embodiments, Escherichia coli Nissle 1917 or any probiotic strain, can be genetically modified or selected through evolution to produce queuine or its precursors at a level higher than the unmodified strain, using techniques such as CRISPR or lambda red recombination. In some embodiments, this modification or selection is characterized by alterations to genes encoding the PreQ1 riboswitch regulatory element, which regulates the bacterial cell's queuine synthesis via a feedback mechanism. In some embodiments, the genes encoding the PreQ1 riboswitch regulatory element are of at least 50% sequence identity to SEQ IDs 90761-91398 (e.g., at least 60% identity, at least 70% identity, at least 80% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity). In some embodiments, the bacteria (e.g., Escherichia coli Nissle 1917) can be modified to express at least one gene involved in queuine biosynthesis or transport such as, but not limited to, FolE, QueD, QueE, QueC, QueF, bTGT, QueA, QueG, QueH, YhhQ, and/or QueT. In some embodiments, the bacteria (e.g., Escherichia coli Nissle 1917) can be modified to express at least one gene encoding an enzyme involved in queuine biosynthesis, with at least 50% amino acid sequence similarity to the representative sequences SEQ ID NOs 3660-82283 (e.g., at least 60% similarity, at least 70% similarity, at least 80% similarity, at least 90% similarity, at least 91% similarity, at least 92% similarity, at least 93% similarity, at least 94% similarity, at least 95% similarity, at least 96% similarity, at least 97% similarity, at least 98% similarity, at least 99% similarity, at least 99.5% similarity, at least 99.9% similarity, or 100% similarity). Enzymes produced by queuine biosynthesis genes from other species of bacteria will catalyze the same reactions as those of the reference or representative enzymes.

In some embodiments, the dose of the therapeutic queuine producing bacteria can comprise 1×10⁴ colony forming units (CFUs), 1×10⁵ CFUs, 1×10⁶ CFUs, 1×10⁷ CFUs, 1×10⁸ CFUs, 1×10⁹ CFUs, 1×10¹⁰ CFUs, 1×10¹¹ CFUs or greater than 1×10¹¹ CFUs of the desired bacteria.

In some embodiments, bacteria are purified prior to incorporation into a composition. For instance, bacteria can be purified so that the population of bacteria is substantially free of other bacteria (e.g., comprises at least 90%, at least 91%, at least 92%, at least 93%, at least 94%, at least 95%, at least 96%, at least 97%, or at least 98%, at least 99% of the specific bacterial strain or strains desired in the composition).

In some embodiments, the composition is a probiotic or a medical food comprising at least one queuine producing bacteria. The bacteria can be administered, for instance, as a probiotic, as a capsule, tablet, caplet, pill, troche, lozenge, powder, and/or granule. The strain can also be formulated as a nutraceutical, conventional food, medical food, or drug. The queuine producing bacteria can also be administered as part of a fecal transplant or via suppository. In some embodiments, the composition is formulated for delivery to the gut, as described further herein. In some embodiments, the composition further comprises a prebiotic.

In some embodiments, prebiotics can be delivered to alter the native microbiome to a state of elevated queuine production. This could include delivery of, but is not limited to, the following prebiotics: amino acids (including arginine, glutamate, and ornithine), biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides, polyamines (such as but not limited to spermidine and putrescine), or any combinations of the above. In some embodiments, the prebiotic(s) are combined with queuine-producing bacteria, human gut queuine producing bacteria, or keystone human gut queuine producing bacteria. In some embodiments, the prebiotics are selected so as not to encourage growth or unwanted activity (e.g. virulence factors) of pathogens.

In some embodiments, the composition or dose unit comprises a pharmaceutically acceptable formulation, including an enteric coating or similar composition to promote survival of or avoid the acidity of the stomach and permit delivery into the small or large intestines.

In some embodiments, the composition further comprises a pharmaceutically acceptable carrier, wherein the one or more isolated non-pathogenic queuine-producing bacterial strains or an isolated product derived therefrom is present in an amount effective to alter queuine levels in a subject in need thereof. Accordingly, in one aspect, described herein is a pharmaceutical composition comprising queuine, an analog, derivative or precursor thereof, or a combination of any of these, in an amount effective to alter queuine levels in a subject in need thereof, and a pharmaceutically acceptable carrier. In some embodiments, the queuine, analog, derivative or precursor is isolated from a queuine-producing bacterial strain or culture medium in which a queuine-producing bacterial strain has been cultured.

In some embodiments, a composition comprising one or more isolated, non-pathogenic queuine-producing bacterial strains or an isolated product derived therefrom as described herein further comprises a different therapeutic composition in an amount effective to treat a CNS disease or disorder, non-limiting examples of which are described further herein.

In some embodiments queuine-producing bacteria are isolated from appropriate samples from where they are predicted to reside. For example, bacteria identified in disclosure as keystone queuine producing bacteria have been isolated from human fecal samples. One skilled in the art would be able to isolate such bacterial taxa from fecal samples using cultivation and identification methods known to trained microbiologists (see e.g., Lagier, J. C. et al. Culturing the human microbiota and culturomics. Nat Rev Microbiol, 540-550, (2018)). These cultivation campaigns can leverage microbiological agars, broths, selective and enrichment conditions (e.g. antibiotics or specific nutrients used by queuine-producing bacteria) to enrich for and/or isolate individual colonies of bacteria. These isolated colonies can be purified and taxonomically identified by 16S rRNA sequencing, to identify which colony or colonies is/are predicted keystone human gut queuine-producing bacteria. These isolates can then be further profiled for suitability as a therapeutic, medical food, or nutraceutical by assessing the presence of desired (e.g., fast growth rates, capability of surviving lyophilization at a recovery >0.1%, capable of growing in commercial manufacturing mediums) and undesired (e.g., history of being a pathogen, antibiotic resistance to clinically relevant antibiotics, mobile elements, toxins, virulence factors, and a strong association with human disease) characteristics.

In some embodiments, additional keystone human gut queuine producing bacteria can be identified by screening a human-derived strain collection for queuine producing bacteria. As one approach, one skilled in the art can culture a diverse panel of human gut bacteria in multiple bacterial mediums (e.g. nutrient rich, nutrient poor, or environmentally similar to the mammalian gastrointestinal tract (e.g. similar pH, nutritional profiles, or presence of other bacteria and their metabolites)), and then measure queuine, queuine-modified RNA, or queuine-related metabolites (e.g. precursors) in the bacterial supernatant or cell pellet via LC/MS following enzymatic digestion, or other appropriate methods. One such method involves migration of select tRNA species through acrylamide electrophoresis gel infused with N-acrolyl-3-aminophenylboronic acid (“APB Gel”), which causes queuosinylated tRNA to form a separate band from unqueuosinylated tRNA (see e.g., Matuszek, Z. a. P., T. Quantification of Queuosine Modification Levels in tRNA from Human Cells Using APB Gel and Northern Blot. Bio-protocol 9, (2019)). In some embodiments, leveraging a collection of queuine producing bacteria and mediums in which they produce queuine (or queuine related metabolites or queuine precursors), one can furthermore identify prebiotics which further enhance queuine production by these bacteria, by comparing levels of queuine (or queuine related metabolites) in cultures with and without the candidate prebiotics. Similarly, one can employ such a method to identify synergistic combinations of queuine producing bacteria (in which the combination results in higher production of queuine than the organisms alone).

In some embodiments, leveraging human microbiome sequencing data, one can identify keystone queuine producing bacteria that co-occur within a mammalian host, using computation methods such as Meta-network and MDiNE (see e.g., Yang et al. Meta-network: optimized species-species network analysis for microbial communities. BMC Genomics 20, 187, (2019); McGregor et al. MDiNE: A model to estimate differential co-occurrence networks in microbiome studies. Bioinformatics, (2019)). Without wishing to be bound by theory, keystone queuine producing bacteria that co-occur with humans have a higher likelihood at elevating host queuine levels, as they are not competing for the same ecological niche. Conversely, keystone queuine producing bacteria that do not co-occur are poor candidates for co-administration, as they likely do compete for the same niche. Such predictions can be further supported by leveraging transcriptomic datasets, in which one looks to identify bacteria that both co-occur and express queuine producing genes within the target mammalian host (e.g. a human).

### Queuine Precursors and Queuine-Related Metabolites

In some embodiments, the present disclosure provides for delivering a composition of queuine, queuine analogs, queuine precursors, queuine-related metabolites, or combinations thereof, to increase queuine levels of or otherwise treat the mammalian host presenting with a queuine-associated mental health disorder or disease. In some embodiments, the present disclosure provides for a method of identifying a mammalian host presenting with a queuine-associated mental health disorder or disease, followed by treating them with a composition of queuine, queuine analogs, queuine precursors, queuine related metabolites, or combinations thereof.

In some embodiments, queuine, queuine analogs, queuine precursors or combinations thereof, are delivered to an individual in need thereof (described below) at a dose of at least 1 g, at least 500 mg, at least 100 mg, at least 10 mg, at least 1000 μg, at least 500 μg, at least 250 μg, or at least 100 μg.

In some embodiments, the composition of queuine, queuine analogs, queuine precursors, queuine related metabolites, or combinations thereof comprise a pharmaceutically acceptable formulation, including an enteric coating or similar composition to promote survival of or avoid the acidity of the stomach and permit delivery into the small or large intestines. In some embodiments, the composition can be delivered as a capsule, tablet, caplet, pill, troche, lozenge, powder, and/or granule. In some embodiments the composition of queuine, queuine analogs, queuine precursors, or combinations thereof can be delivered intravenously, through a patch, or in a slow release format. The composition can also be formulated as a nutraceutical, conventional food, medical food, or drug.

In some embodiments, the queuine analogs include structural variants of queuine or a molecule or macromolecule comprising queuine or structural variants thereof that retains one or more activities of queuine (e.g., tRNA-queuosine translation, regeneration of BH4, synthesis of monoamine neurotransmitters, etc.). Non-limiting examples of queuine structural variants are described further herein. In some embodiments, the queuine analogs are in a form adapted for oral use or administration. In particular, a queuine analog can be part of a covalent or ionic complex, such as queuosine, a mannosyl queuosine, galactosyl queuosine, or a glutamyl queuosine. It can also be administered in the form of tRNA-queuosine or an oligonucleotide comprising queuosine. Among these derivatives are the glycosylated derivatives of queuine and queuosine, such as mannosylqueuine, galactosylqueuine, and aminoacylated derivatives such as glutamylqueuine.

In some embodiments a queuine precursor refers, for example, to its intermediate precursor, epoxyqueuine, whether in free form or in the form of a covalent complex with molecules or macromolecules. In some embodiments, a precursor of queuine refers to any molecule listed in FIG. 3. In some embodiments precursors of queuine includes cofactors required for queuine biosynthesis in bacteria, such as Vitamin B-12, generally referred to as cobalamin, which is required for a functional QueG. Cobalamin is one of the most complex small molecules found in nature. Vitamin B-12 is a cofactor with four pyrrole rings that has a central cobalt ion bonded to four equatorial nitrogen ligands from the corrin ring. Uroporphyrinogen III (Uro III) is a precursor of cobalamins. The first part of the biosynthetic pathway for cobalamin involves the conversion of Uro III to coenzyme B12 intermediate cobinamide (cobI), followed by the synthesis of dimethylbenzimidazole (Dmb) from flavin precursors, concluding with covalent joining of cobI with Dmb and a phosphoribosyl group (see e.g., Roth, Lawrence, & Bobik, Cobalamin (coenzyme B12): synthesis and biological significance. Annu Rev Microbiol 50, 137-181, (1996); Lawrence & Roth, Evolution of coenzyme B12 synthesis among enteric bacteria: evidence for loss and reacquisition of a multigene complex. Genetics 142, 11-24 (1996)). There are various naturally occurring analogs of cobalamin that comprise a hydroxyl (—OH), methyl (—CH3), or a 5′-deoxyadenosyl group, such as methylcobalamin, hydroxocobalamin, and 5-deoxyadenosylcobalamin, including those that are found in human fecal samples. Without wishing to be bound by theory, it is likely that multiple analogs of cobalamin can be used as a cofactor for QueG in the mammalian gastrointestinal tract, and can thus influence queuine biosynthesis.

In some embodiments, a queuine-related metabolite refers to any metabolite directly or indirectly influenced by queuine levels in the host. In some embodiments, biopterins (particularly BH2+BH4); monoamine neurotransmitters; imidazoleamines, e.g. histamine; catecholamines, e.g. adrenaline (epinephrine), dopamine, noradrenaline (norepinephrine); indolamines, e.g. serotonin (5-HT), melatonin, nitric oxide, phenylalanine, tyrosine, tryptophan, kynurenine, kynurenic acid, quinolinic acid, picolinic acid, melanin, or combinations thereof, are queuine-related metabolites. In some embodiments, queuine-related metabolites can be high or low in a target population (e.g. high kynurenine is likely considered an indicator of poor functional serotonin biosynthesis).

### Queuine Analogs and Derivatives

It is specifically contemplated that one or more queuine structural analogs or derivatives that share the functional properties with queuine can be used in a method as described herein in place of, or in addition to queuine.

In some embodiments, a queuine-related analog or derivative can comprise compounds of formula (II), shown below, and pharmaceutically acceptable salts and solvates thereof.

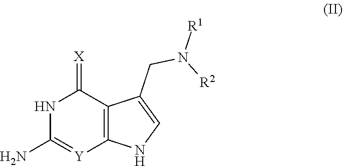

In some embodiments, R¹ is selected from H and CH₃. In some embodiments, R² is selected from H, C₄H₉ alkyl, C₆H₁₃ alkyl and C₃H₆-phenyl, said Phenyl optionally substituted by OH or OCH₃, X is O or SY is C, N or S. Preferably alkyl chains are straight chain. In a preferred embodiment, R¹ is H. In a preferred embodiment R² is selected from C₄H₉ alkyl, C₆H₁₃ alkyl and C₃H₆-phenyl. In a particularly preferred embodiment R² is C₃H₆-phenyl. In a preferred embodiment X is O. In a preferred embodiment Y is C or N. In a particularly preferred embodiment Y is N. Particularly preferred are those compounds of formula (II) where: X is OY is NR¹ is H; and R² is selected from C₄H₉ alkyl, C₆H₁₃ alkyl and C₃H₆-phenyl. In a particularly preferred embodiment R² is C₃H₆-phenyl

Particularly preferred compounds comprising queuine analogs or derivatives include but are not limited to: (a) 2-amino-5-((butylamino)methyl)-3,7-dihydro-4H-pyrrolo[2,3-d]pyrimidin-4-one; (b) N-((2-amino-4-oxo-4,7-dihydro-3H-pyrrolo[2,3-d]pyrimidin-5-yl)methyl)butan-1-aminium chloride; (c) 2-amino-5-((hexylamino)methyl)-3,7-dihydro-4H-pyrrolo[2,3-d]pyrimidin-4-one; (d) N-((2-amino-4-oxo-4,7-dihydro-3H-pyrrolo[2,3-d]pyrimidin-5-yl)methyl)hexan-1-aminium chloride; (e) Queuine, 2-amino-5-((((1S,4S,5R)-4,5-dihydroxycyclopent-2-en-1-yl)amino)methyl)-3,7-dihydro-4H-pyrrolo[2,3-d]pyrimidin-4-one; (f) Queuine HCl 2-Amino-5-[[[(1S,4S,5R)-4,5-dihydroxy-2-cyclopenten-1-yl]amino]methyl]-1,7-dihydro-4H-pyrrolo[2,3-d]pyrimidin-4-one, monohydrochloride; (g) 2-amino-5-(((3-phenylpropyl)amino)methyl)-3,7-dihydro-4H-pyrrolo[2,3-d]pyrimidin-4-one; or (h) N-((2-amino-4-oxo-4,7-dihydro-3H-pyrrolo[2,3-d]pyrimidin-5-yl)methyl)-3-phenylpropan-1-aminium chloride.

Suitable salts include salts of acidic or basic groups present in compounds of formula (II). The compounds of formula (II) that are basic in nature are capable of forming a wide variety of salts with various inorganic and organic acids. The acids that may be used to prepare pharmaceutically acceptable acid addition salts of such basic compounds of formula (II) are those that form non-toxic acid addition salts. Suitable salts include acetate, benzenesulfonate, benzoate, bicarbonate, bisulfate, bitartrate, borate, bromide, calcium edentate, camsylate, carbonate, chloride, clavulanate, citrate, dihydrochloride edentate, edisylate, estolate, esylate, ethylsuccinate, fumarate, gluceptate, gluconate, glutamate, glycollylarsanilate, hexylresorcinate, hydrabamine, hydrobromide, hydrochloride, iodide isothionate, lactate, lactobionate, laurate, malate, maleate, mandelate, mesylate, methylsulfate, mucate, napsylate, nitrate, oleate, oxalate, pamoate, palmitate, pantothenate, phosphate, diphosphate, polygalacturonate, salicylate, stearate, subacetate, succinate, tannate, tartrate, teoclate, tosylate and valerate salts.

In some embodiments, a queuine-related analog or derivative can comprise compounds of formula (III), shown below, and pharmaceutically acceptable salts and solvates thereof.

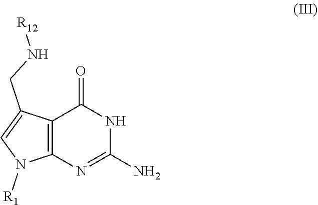

In some embodiments, R1 represents —H or a ribosyl group of formula (IV).

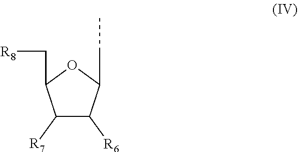

In some embodiments, R₆ represents —H; —O—R₉ or —O—CO—R₉ wherein R₉ is H, an alkyl group having from 1 to 6 carbon atoms or an aryl group having from 3 to 12 carbon atoms. In some embodiments, R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group. In some embodiments, R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group. In some embodiments, R₁₂ represents a saturated or unsaturated alkyl, cycloalkyl, heterocycloalkyl or ether group having from 1 to 20 carbon atoms, optionally substituted by at least one group selected from the group consisting of: (1) an alkyl group having from 1 to 20 carbon atoms, (2) an aryl or heteroaryl group having from 3 to 20 carbon atoms, (3) a cycloalkyl or heterocycloalkyl group having from 3 to 20 carbon atoms, (4) a hydroxyl group, (5) a carbonyl or carboxyl group having from 1 to 20 carbon atoms, (6) an epoxy group, (7) an —O—R₄ group wherein R₄ is H, an alkyl group having from 1 to 6 carbon atoms, an aryl group having from 3 to 12 carbon atoms, a glycosyl group or an aminoacyl group, and (8) an —O—CO—R₅ group wherein R₅ is an alkyl group having from 1 to 6 carbon atoms, an aryl group having from 3 to 12 carbon atoms or a glycosyl group;

or a pharmaceutically acceptable salt or hydrate thereof,
for use in the prevention or treatment of a disease associated with a mitochondrial dysfunction in an individual.

In some embodiments, a queuine-related analog or derivative can comprise compounds of formula (V), shown below, and pharmaceutically acceptable salts and solvates thereof.

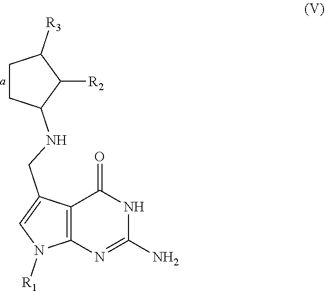

In some embodiments, a represents a double bond or an epoxy group, and R₁ represents —H or a ribosyl group of formula (VI).

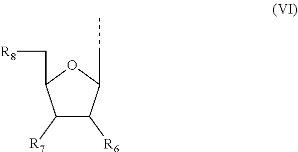

In some embodiments, R₆ represents —H; —O—R₉ or —O—CO—R₉ wherein R₉ is H, an alkyl group having from 1 to 6 carbon atoms or an aryl group having from 3 to 12 carbon atoms. In some embodiments, R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group. In some embodiments, R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group. In some embodiments, R₂ and R₃, which are identical or different, represent —O—R₄ wherein R₄ is H, an alkyl group having from 1 to 6 carbon atoms, an aryl group having from 3 to 12 carbon atoms, a glycosyl group or an aminoacyl group; or —O—CO—R₅ wherein R₅ is an alkyl group having from 1 to 6 carbon atoms, an aryl group having from 3 to 12 carbon atoms or a glycosyl group. Additional queuine analogs or derivatives are described, for example, in U.S. patent applications US20170240553A1 and US20190224174A1, the contents of which are incorporated herein by reference in their entireties.

### Queuine-Associated Diseases or Disorders

The queuine-associated compositions described herein can be administered to a patient in need thereof, for instance for the treatment of a mental illness or disease associated with low levels of queuine (“queuine-associated mental illness or disease”). Described here are methods of use for such compositions.

In one aspect, described herein is a method of increasing queuine levels in a subject in need thereof, the method comprising administering to the subject a composition as described herein in an amount effective to increase queuine levels in the subject. In some embodiments, the subject is a mammalian subject. In some embodiments, the subject is a human subject.

In one or more embodiments of any of the above-aspects, the queuine associated mental illness or disease (also referred to herein as a central nervous system (CNS) disorder associated with queuine deficiency) that can be treated by administration of a composition described herein is selected from the group consisting of: clinical depression, bipolar disorder, schizophrenia, anxiety, anxiety disorders, addiction, social phobia, major depressive disorder, treatment-resistant major depressive disorder (TR-MDD), major depressive disorder and its subtypes (melancholic depression, atypical depression, catatonic depression, postpartum depression, and seasonal affective disorder), Neurodegenerative amyloid disorders (Parkinson's, Alzheimer's, and Huntington's diseases), restless leg syndrome, neuropathic pain, pain disorders, dementia, epilepsy, stiff-person syndrome, premenstrual dysphoric disorder, autism spectrum disorders, sleep disorders, obsessive-compulsive disorder, Tourette's syndrome, intellectual disability, Pediatric Autoimmune Neuropsychiatric Disorders Associated with Streptococcal infections (PANDAS), post-treatment Lyme disease syndrome, and attention deficit hyperactivity disorder (ADHD).

In some embodiments, the method further comprises improving at least one symptom or etiologically linked comorbidity of a queuine associated mental disorder or disease in the subject selected from the group consisting of: anhedonia, fatigue, insomnia, motor dysfunction, stress, persistent anxiety, persistent sadness, social withdrawal, substance withdrawal, irritability, thoughts of suicide, thoughts of self-harm, restlessness, low sex drive, lack of focus, loss of appetite, seizures, memory loss, anger, bouts of emotional reactivity, confusion, pain, cardiovascular or erectile dysfunction caused by biopterin-linked nitric oxide synthesis defects, and muscle spasms.

### Methods of Identifying Queuine-Deficient Mammals

In some embodiments, the method of treatment can comprise first diagnosing a subject or patient who can benefit from treatment by a composition described herein. In some embodiments, the method further comprises administering to the patient a composition described herein.

In some embodiments, the process of identifying a subject with a queuine associated mental illness or disease can be carried out by a trained psychologist, psychiatrist, or neurologist. For instance, a psychiatrist, psychologist, or neurologist can diagnose a subject with a mental illness or disease of the central nervous system by evaluating the subject's behavior for symptoms of the mental illness or disease. One of skill in the art will understand that mental illness can also be identified in a subject with the aid of the Diagnostic and Statistical Manual of Mental Disorders (DSM-5), (American Psychiatric Association), or other relevant tools.

In one or more embodiments, the process of identifying a subject with a queuine associated mental illness or disease can comprise diagnosing the subject with a mental illness or disease. In some embodiments, the mental illness or disease is identified or diagnosed using fMRI. In some embodiments, mental illness or disease can be identified with standard psychological and neurological surveys, or in other methods known to experts in the field.

In some embodiments, a subject in need of treatment with a composition described herein can be identified by identifying low levels of queuine, queuine-incorporated RNA, queuine precursors, or queuine-related metabolites in the subject's blood, liver, brain, serum, stool, or other bodily fluid or tissue. In some embodiments, the percentage of tRNAAsp/His/Tyr/Asn in the subject's blood, liver, brain, serum, stool, or other bodily fluid or tissue with a queuosine moiety in the first position of the anticodon is below about 80%. In some embodiments, the percentage of tRNAAsp/His/Tyr/Asn in the subject's blood, liver, brain, serum, stool, or other bodily fluid or tissue with a queuosine moiety in the first position of the anticodon is below 80%, below 70%, below 60%, below 50%, below 40%, below 30%, below 20%, or below 10%. In some embodiments, the amount of queuine, queuine precursors, or queuine related metabolites in the subject's blood, liver, brain, serum, stool, or other bodily fluid or tissue (e.g., the initial amount of queuine in the subject's blood, liver, brain, serum, stool, or other bodily fluid or tissue) is below 200 ng, 100 ng, below 50 ng, below 25 ng, below 20 ng, below 15 ng, below 10 ng, below 9 ng, below 8 ng, below 7 ng, below 6 ng, below 5 ng, below 4 ng, below 3 ng, below 2 ng, below 1 ng, below 0.5 ng, below 0.1 ng, below 0.01 ng, or below 0.001 ng of queuine, queuine precursors, or queuine-related metabolites per gram or mL sample or tissue (e.g., as measured by LC/MS or other appropriate methods). In some embodiments, the amount of queuine, queuine-incorporated RNA, queuine precursors, or queuine related metabolites in the subject's blood, liver, brain, serum, stool, or other bodily fluid or tissue (e.g., the initial amount of queuine in the subject's blood, liver, brain, serum, stool, or other bodily fluid or tissue) is at a level less than 1.5 or more standard deviations from what is detected in a healthy person. In some embodiments, the amount of queuine or related metabolites in the prefrontal cortex, or other areas of the brain, is below about 100 uM, below 50 uM, below 25 uM, below 20 uM, below 15 uM, below 10 uM, below 9 uM, below 8 uM, below 7 uM, below 6 uM, below 5 uM, below 4 uM, below 3 uM, below 2 uM, below 1 uM, below 0.5 uM, below 0.1 uM, below 0.01 uM, or below 0.001 uM, e.g., as measured by proton magnetic resonance (PMR), or another similar technique.

In some embodiments, a subject in need of treatment with a composition described herein can be identified by determining levels of queuine producing bacteria in a given tissue (e.g. stool). In some embodiments the percentage of queuine producing bacteria, human gut queuine producing bacteria, or keystone queuine producing bacteria in the subject's gut (e.g., the initial amount) represents less than about 10% of total 16S rRNA sequences as measured by sequencing using such methods as 16S rRNA gene Illumina™ sequencing or quantitative PCR. In some embodiments, the percentage of queuine producing bacteria, human gut queuine producing bacteria, or keystone queuine producing bacteria in the subject's gut represents about 9%, about 8%, about 7%, about 6%, about 5%, about 4%, about 3%, about 2%, about 1%, or less than about 1% of the total 16S sequences measured in the subject's gut.

In some embodiments, a subject in need of treatment with a composition described herein can be diagnosed as being dysbiotic or in need of queuine supplementation by analysis of blood or tissue for the amounts or ratios of tRNAHis/Asp/Tyr/Asn which contain(s) queuosine or a glycosylated queuosine derivative in the “wobble” position (position 34) of the anticodon, rather than guanosine. In a non-limiting example, a queuine deficiency would be diagnosed by a finding of a percentage of queuosine-modified Histidyl tRNA in a sample (e.g., preferably liver or alternatively blood, brain, serum, or stool) of less than 40%, less than 50%, less than 60%, less than 70%, less than 80%, less than 90%, or less than 95% out of the total Histidyl tRNA in the sample.

In some embodiments of any of the above aspects, the amount of queuine, queuine-incorporated RNA, queuine precursors, or queuine-related metabolites in the subject's blood, liver, brain, serum, stool, or other bodily fluid or tissue is increased following administration of a treatment as described herein by at least 0.1%, 1%, 5%, 10%, 20%, 30%, 40%, 50%, 60%, 70%, 80%, 90%, 100%, 500%, 1000%, 2000%, 3000%, 4000%, 5000%, or more relative to the initial amount in the subject's samples (e.g., as measured with LC/MS or other appropriate methods known to those familiar with the field). In some embodiments, at least one queuine producing bacteria, human gut queuine producing bacteria, or keystone queuine producing bacteria is increased 0.1%, 1%, 5%, 10%, 20%, 30%, 40%, 50%, 60%, 70%, 80%, 90%, 100%, 500%, 1000%, 2000%, 3000%, 4000%, 5000%, or more in the subject's stool relative to the initial amount in the subject's sample (e.g., as measured by qPCR, next generation sequencing, or other appropriate methods known to those familiar with the field). In some embodiments, the level of expression of at least one queuine producing enzyme is increased 0.1%, 1%, 5%, 10%, 20%, 30%, 40%, 50%, 60%, 70%, 80%, 90%, 100%, 500%, 1000%, 2000%, 3000%, 4000%, 5000%, or more in the subject's sample relative to the initial level of expression in the subject's sample (e.g., as measured by qPCR, next generation sequencing, or other appropriate methods known to those familiar with the field).

In some embodiments, a subject in need of treatment with a composition as described herein can be identified by having a mutation in one or more genes involved in queuine salvaging. In some embodiments, queuine salvaging genes include, but are not limited to, Queuine TRNA-Ribosyltransferase Catalytic Subunit 1 (QTRT1) and QTRT2 (previously called QTRTD1), or DUF2419 (domain of unknown function 2419). In some embodiments, a subject in need of treatment with a composition described herein can be identified by having high or low expression of queuine salvaging genes, such as QTRT1 and QTRT2 (previously called QTRTD1), or DUF2419, in target issues. See e.g., Zallot et al., Plant, Animal, and Fungal Micronutrient Queuosine Is Salvaged by Members of the DUF2419 Protein Family, ACS Chem Biol. 2014 Aug. 15; 9(8): 1812-1825.

In some embodiments, a subject in need of treatment with a composition described herein can be identified by detecting low levels of queuine-producing bacteria (e.g., comprising 16S from SEQ ID NO: 1-78, 1-406, or 1-3659) in the subject's stool (e.g., using quantitative next-generation 16S sequencing). In some embodiments, the level of queuine-producing bacteria in the subject's stool is below about 80% that of a healthy control. In some embodiments, the level of queuine-producing bacteria in the subject's stool is below 80%, below 70%, below 60%, below 50%, below 40%, below 30%, below 20%, or below 10% that of a healthy control.

In some embodiments, a subject in need of treatment with a composition described herein can be identified by detecting low DNA, RNA, or protein levels associated with at least one queuine biosynthesis enzyme (e.g., SEQ ID NO: 3660-82283) or low DNA or RNA levels of at least one PreQ1 riboswitch regulatory element, which regulates the bacterial cell's queuine synthesis via a feedback mechanism (e.g., SEQ ID NO: 90761-91398) in the subject's stool (e.g., using whole-genome sequencing or gene-specific sequencing to detect the nucleic acids encoding the enzymes, or through proteomic analysis such as LC MS). In some embodiments, the level of at least one queuine biosynthesis enzyme or at least one PreQ1 riboswitch in the subject's stool is below about 80% that of a healthy control. In some embodiments, the level of at least one queuine biosynthesis enzyme or at least one PreQ1 riboswitch in the subject's stool is below 80%, below 70%, below 60%, below 50%, below 40%, below 30%, below 20%, or below 10% that of a healthy control.

Accordingly, the present disclosure provides for the treatment of queuine-associated mental illness or disease comprising administering to the subject one or more queuine-producing bacterial strains (e.g., purified strains) and/or their derivatives (e.g. live bacteria, dead bacteria, spent medium(s) derived from a bacteria, cell pellet(s) of a bacteria, purified metabolite(s) produced by bacteria, purified protein(s) produced by a bacteria, or combinations thereof), queuine, queuine precursors, queuine analogs, queuine-related metabolites, prebiotics, and/or compositions comprising the same for administration to a subject in need thereof.

### Endozepines

Another exemplary CNS metabolic pathway is illustrated in FIG. 1, panel A, which depicts the pathway of endozepine production. Endozepines are endogenous benzodiazepine receptor ligands, and as such are involved in a variety of neural processes; see e.g., Farzampour et al., Endozepines, Adv Pharmacol. 2015; 72: 147-164. Levels of endozepines in the mammalian brain are described herein as being influenced at least in part by enteric microbes, and certain CNS disorders amenable to treatment using the methods and compositions described herein are associated with dysbiosis characterized by loss or impairment of these microbes (selected by their ability to direct synthesis of organochlorine precursors implicated in endozepine synthesis).

### Endozepine Producing Bacteria or Yeast

In some embodiments, the present disclosure provides one or more non-pathogenic endozepine producing bacterial or yeast strains (e.g., purified strains) and/or their derivatives (e.g. live bacteria or yeast, dead bacteria or yeast, spent medium(s) derived from a bacteria or yeast, cell pellet(s) of a bacteria or yeast, purified metabolite(s) produced by bacteria or yeast, purified protein(s) produced by a bacteria or yeast, or combinations thereof) and compositions comprising the same for administration to a subject in need thereof. The bacteria or yeast can be naturally occurring, or can be engineered (e.g., through strain engineering or selection) to produce one or more endozepines. In some embodiments, one strain of endozepine producing bacteria or yeast can be administered to a subject in need thereof. In some embodiments, multiple strains of endozepine producing bacteria or yeast can be administered to a subject in need thereof. In some embodiments, the one or more bacteria or yeast (e.g., purified bacteria or yeast) can act synergistically. For instance, the multiple bacteria or yeast can act synergistically to produce high levels of at least one endozepine, via, including but not limited to, cross feeding of nutrients important for endozepine biosynthesis or via supporting growth or survival of endozepine producing bacteria or yeast. Accordingly, any one, or any combination of the endozepine producing bacteria or yeast described herein can be administered to a subject in need thereof.

In some embodiments, the bacteria or yeast described herein can produce at least one endozepine under physiologically relevant conditions, such as under the conditions of the human gut. In some embodiments, a pH relevant to the human gut is between about 4.5 and about 7.5. For instance, the pH can be about 4.5, 5.0, 5.5., 6.0, 6.5, 7.0 7.5, or any value between about 4.5 and 7.5. In some embodiments, the physiologically relevant conditions of the human gut include being exposed to carbon, nitrogen, or micronutrients found in the human gut (such as host-derived glycoproteins like mucin) or those in a typical human diet (e.g. complex or simple glycans).

In some embodiments, endozepine producing bacteria or yeast are identified by the presence of genes involved in endozepine biosynthesis (see e.g., Table 2), using genome sequencing, qPCR, or other related methods. In some embodiments, a gene involved in endozepine biosynthesis is tryptophan halogenase. In some embodiments, one endozepine biosynthesis gene is functionally similar to the pyrroloquinoline quinone precursor peptide synthesis gene pqqA, with the representative sequences of SEQ ID NOs: 95292-95321. In some embodiments, the pqqA-related gene is characterized by a conservative substitution at positions 16-20 of the classical pqqA gene, resulting in condensation of modified quinoline derivatives which function as endozepines or endozepine precursors. In some embodiments, the endozepine biosynthesis genes have a high percent identity to those involved in the biosynthesis of the putative endozepines Sibiromycin, Tomaymycin, and Viridicatin (see e.g., Table 2). In some embodiments, an endozepine producing bacteria or yeast is classified as such by having 1, 2, 3, 4, or more of the genes involved in biosynthesis of Sibiromycin, Tomaymycin, Viridicatin, or other endozepines. In some embodiments, the genes encoding an enzyme involved in endozepine biosynthesis are of at least 50% amino acid sequence similarity with the representative sequences of SEQ ID NOs: 82284-90760 or SEQ ID NOs: 95292-95321 (e.g., at least 60% similarity, at least 70% similarity, at least 80% similarity, at least 90% similarity, at least 91% similarity, at least 92% similarity, at least 93% similarity, at least 94% similarity, at least 95% similarity, at least 96% similarity, at least 97% similarity, at least 98% similarity, at least 99% similarity, at least 99.5% similarity, at least 99.9% similarity, or 100% similarity). Enzymes produced by endozepine biosynthesis genes from other species of bacteria or yeast will catalyze the same reactions as those of the reference or representative enzymes.

In some embodiments, the endozepine producing bacteria or yeast are related taxonomically to known or putative producers of endozepine precursors including benzodiazepine, quinoline, and quinazoline derivatives. In some embodiments, they belong to the phylum Proteobacteria, especially the class Betaproteobacteria or Gammaproteobacteria. In some embodiments, they have a 16S sequence with a substantial percent identity to that of SEQ ID NOs: 91404-91406 or SEQ ID NOs: 95264-95291. In some embodiments, the endozepine producing bacteria or yeast can have at least 90% 16S or 18S sequence identity to any of the 16S or 18S sequences listed in SEQ ID NOs 91404-91406 or SEQ ID NOs: 95264-95291 (e.g., at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity).

In some embodiments, the human gut endozepine producing bacteria or yeast are non-pathogenic bacteria or yeast belonging to a genus selected from the group consisting of: Bacillus, Streptomyces, Emericella, and Aspergillus. In some embodiments, the human gut endozepine producing bacteria or yeast are non-pathogenic bacteria or yeast belonging to a species selected from the group consisting of: Bacillus subtilis, Streptomyces, Emericella nidulans (also known as Aspergillus nidulans). In some embodiments, the human gut endozepine producing bacteria or yeast are non-pathogenic bacteria or yeast belonging to a strain selected from the group consisting of: Bacillus subtilis subsp. natto, Streptomyces achromogenes strain E91CS4, and Emericella nidulans strain BAB-2648.

In some embodiments, the composition further comprises a pharmaceutically acceptable carrier, wherein the one or more isolated non-pathogenic endozepine-producing bacterial or yeast strains or an isolated product derived therefrom is present in an amount effective to alter endozepine levels in a subject in need thereof. Accordingly, in one aspect, described herein is a pharmaceutical composition comprising endozepine, an analog, derivative or precursor thereof, or a combination of any of these, in an amount effective to alter endozepine levels in a subject in need thereof, and a pharmaceutically acceptable carrier. In some embodiments, the endozepine, analog, derivative or precursor is isolated from an endozepine-producing bacterial or yeast strain or culture medium in which a queuine-producing bacterial or yeast strain has been cultured.

In some embodiments, a composition comprising one or more isolated, non-pathogenic endozepine-producing bacterial strains or an isolated product derived therefrom as described herein further comprises a different therapeutic composition in an amount effective to treat a CNS disease or disorder, non-limiting examples of which are described further herein.

In some embodiments, endozepine producing bacteria or yeast are identified by growing isolated mammalian bacteria or yeast (e.g. human fecal bacteria or yeast) in multiple bacterial or yeast mediums (e.g. nutrient rich, nutrient poor, or environmentally similar to the mammalian gastrointestinal tract (e.g. similar pH or nutritional profiles)), and then measuring endozepine levels in the supernatants or cell pellets via LC/MS or other appropriate methods (e.g. a fluorescent protein-based assay to measure GABAA channel activation and allosteric modulation in mammalian cells, see e.g., Johansson et al. PLoS One 8, e59429, (2013)). In some embodiments, leveraging a collection of such identified endozepine producing bacteria or yeast and mediums in which they produce endozepines, one can furthermore identify prebiotics which further enhance endozepine production by these bacteria or yeast, by comparing levels of endozepines in cultures with and without the candidate prebiotics. Similarly, one can employ such a method to identify synergistic combinations of endozepine producing bacteria or yeast (where the combination results in higher production of endozepines than the organisms alone). Without wishing to be bound by theory, if unknown, one can also then identify bacterial or yeast genetic elements associated with endozepine biosynthesis by creating CRISPR or transposon gene knockout libraries of endozepine producing bacteria or yeast, and screening those libraries for clones that no longer produce endozepines. Those clones can then have the disrupted gene/sequence identified, which can be leveraged to predict other endozepine producing bacteria or yeast. Similarly, one can leverage a “gain-of-function” strategy, in which DNA from an endozepine producing bacteria or yeast is inserted into a genetically malleable host organism such as Escherichia coli or Saccharomyces cerevisiae or Schizosaccharomyces pombe in an ordered or non-ordered way, and the recombinant Escherichia coli or S. cerevisiae or S. pombe clones are then screened for production of endozepines. Such a technique has recently been employed to identify genes involved in bacterial xenobiotic metabolism; see e.g., Zimmermann et al. Nature 570, 462-467, (2019).

In some embodiments, keystone endozepine producing bacteria or yeast are then identified in the target mammalian species (e.g. a human) by mining or generating fecal, colonic wash, or intestinal biopsy transcriptome cohorts to identify which bacteria or yeast within a target mammal expresses genes involved in endozepine biosynthesis (either known, or those identified in the CRISPR or transposon library knockout strategy, or “gain-of-function” strategy, described above).

In some embodiments, the endozepine producing bacteria or yeast can be further profiled for suitability as a therapeutic, medical food, or nutraceutical by assessing the presence of wanted (e.g., fast growth rates, capability of surviving lyophilization at a recovery >0.1%, capable of growing in commercial manufacturing mediums) and unwanted (e.g., history of being a pathogen, antibiotic resistance to clinically relevant antibiotics, mobile elements, toxins, virulence factors, and a strong association with human disease) characteristics.

In some embodiments, such genes involved in endozepine biosynthesis can be introduced into a host probiotic such as Escherichia coli Nissle 1917, where it expresses said genes to produce endozepines constitutively or inducibly.

In some embodiments, purified endozepines (or synthetically produced analogs) from endozepine producing bacteria or yeast can be administered as a drug, medical food, or nutraceutical.

In some embodiments, the endozepine-related compositions can function in places beyond the brain, such as the peripheral or enteric nervous systems. Without wishing to be bound by theory, this can be useful for conditions presenting with disrupted intestinal motility, pain, inflammation, or metabolic features.

### Endozepine-Associated Diseases and Disorders

The endozepine compositions described herein can be administered to a patient in need thereof, for instance for the treatment of a mental illness or disease associated with low levels of endozepine (“endozepine-associated mental illness or disease”, also referred to herein as a central nervous system (CNS) disorder associated with endozepine deficiency). Described herein are methods of use for such compositions.

In one aspect, described herein is a method of increasing endozepine levels in a subject in need thereof, the method comprising administering to the subject a composition as described herein in an amount effective to increase endozepine levels in the subject. In some embodiments, the subject is a mammalian subject. In some embodiments, the subject is a human subject.

In one or more embodiments of any of the above-aspects, the endozepine associated mental illness or disease that can be treated by administration of a composition described herein is selected from the group consisting of: depression, bipolar disorder, schizophrenia, anxiety, anxiety disorders, addiction, social phobia, major depressive disorder, treatment-resistant major depressive disorder (TR-MDD), major depressive disorder and its subtypes (melancholic depression, atypical depression, catatonic depression, postpartum depression, and seasonal affective disorder), Neurodegenerative amyloid disorders (Parkinson's, Alzheimer's, and Huntington's diseases) orthostatic tremor, Lafora disease, restless leg syndrome, neuropathic pain, pain disorders, dementia, epilepsy, stiff-person syndrome, premenstrual dysphoric disorder, autism spectrum disorder, sleep disorders, and attention deficit hyperactivity disorder (ADHD).

In some embodiments, the method further comprises decreasing at least one symptom of an endozepine associated mental disorder or disease in the subject selected from the group consisting of: fatigue, insomnia, motor dysfunction, stress, persistent anxiety, persistent sadness, social withdrawal, substance withdrawal, irritability, thoughts of suicide, thoughts of self-harm, restlessness, low sex drive, lack of focus, loss of appetite, seizures, memory loss, anger, bouts of emotional reactivity, confusion, pain, and muscle spasms.

### Methods of Identifying Endozepine Deficient Mammals

In some embodiments, the method of treatment can comprise first diagnosing a subject or patient who can benefit from treatment by a composition described herein. In some embodiments, the method further comprises administering to the patient a composition described herein.

In some embodiments, the process of identifying a subject with a mental illness or disease can be carried out by a trained psychologist, psychiatrist, or neurologist. For instance, a psychiatrist, psychologist, or neurologist can diagnose a subject with a mental illness or disease of the central nervous system by evaluating the subject's behavior for symptoms of the mental illness or disease. One of skill in the art will understand that mental illness can also be identified in a subject with the aid of the Diagnostic and Statistical Manual of Mental Disorders (DSM-5), (American Psychiatric Association), or other relevant tools.

In one or more embodiments, the process of identifying a subject with a mental illness or disease can comprise diagnosing the subject with a mental illness or disease. In some embodiments, the mental illness or disease is identified or diagnosed using fMRI. In some embodiments, mental illness or disease can be identified with standard psychological and neurological surveys, or in other methods known to experts in the field.

In some embodiments, a subject in need of treatment with a composition described herein can be identified by identifying low levels of at least one endozepine in the subject's blood, liver, brain, serum, stool, or other bodily fluid or tissue. In some embodiments, the amount of at least one endozepine in the subject's blood, liver, brain, serum, stool, or other bodily fluid or tissue (e.g., the initial amount of at least one endozepine in the subject's blood, liver, brain, serum, stool, or other bodily fluid or tissue) is below about 100 pg/mL of sample or tissue (e.g., as measured by LC/MS or other appropriate methods). In some embodiments, the amount of at least one endozepine in the subject's blood, liver, brain, serum, stool, or other bodily fluid or tissue (e.g., the initial amount of at least one endozepine in the subject's blood, liver, brain, serum, stool, or other bodily fluid or tissue) is below 10,000 pg/mL, below 1,000 pg/mL, below 500 pg/mL, below 100 pg/mL, below 50 pg/mL, below 10 pg/mL, below 1 pg/mL, or below 0.1 pg/mL of sample or tissue (e.g., as measured by LC/MS, proton magnetic resonance (PMR) or other appropriate methods).

In some embodiments, the percentage of endozepine producing bacteria or yeast (e.g., the initial amount) represents less than about 10% of total 16S or non-human 18S rRNA sequences as measured by sequencing using such methods as 16S or 18S rRNA gene Illumina™ sequencing or quantitative PCR. In some embodiments, the percentage of endozepine producing bacteria or yeast, human gut endozepine producing bacteria or yeast or keystone endozepine producing bacteria in the subject's gut represents about 9%, about 8%, about 7%, about 6%, about 5%, about 4%, about 3%, about 2%, about 1%, or less than about 1% of the total 16S or non-human 18S sequences measured in the subject's gut.

In some embodiments of any of the above aspects, the amount of at least one endozepine in the subject's blood, liver, brain, serum, stool, or other bodily fluid or tissue is increased following administration of a treatment as described herein by at least 0.1%, 1%, 5%, 10%, 20%, 30%, 40%, 50%, 60%, 70%, 80%, 90%, 100%, 500%, 1000%, 2000%, 3000%, 4000%, 5000%, or more relative to the initial amount in the subject's samples (e.g., as measured by LC/MS, proton magnetic resonance (PMR) or other appropriate methods). In some embodiments, at least one endozepine producing bacteria or yeast is increased 0.1%, 1%, 5%, 10%, 20%, 30%, 40%, 50%, 60%, 70%, 80%, 90%, 100%, 500%, 1000%, 2000%, 3000%, 4000%, 5000%, or more in the subject's stool relative to the initial amount in the subject's sample (e.g., as measured by qPCR, next generation sequencing, or other appropriate methods known to those familiar with the field). In some embodiments, the level of expression of at least one endozepine producing enzyme is increased 0.1%, 1%, 5%, 10%, 20%, 30%, 40%, 50%, 60%, 70%, 80%, 90%, 100%, 500%, 1000%, 2000%, 3000%, 4000%, 5000%, or more in the subject's sample relative to the initial level of expression of endozepine enzymes in the subject's sample (e.g., as measured by qPCR, next generation sequencing, or other appropriate methods known to those familiar with the field).

In some embodiments, a subject in need of treatment with a composition described herein can be identified by detecting low levels of endozepine-producing bacteria or yeast (e.g., comprising 16S or 18S from SEQ ID NO: 91404-91406 or 95292-95321) in the subject's stool (e.g., using quantitative next-generation 16S or 18S sequencing). In some embodiments, the level of endozepine-producing bacteria or yeast in the subject's stool is below about 80% that of a healthy control. In some embodiments, the level of endozepine-producing bacteria or yeast in the subject's stool is below 80%, below 70%, below 60%, below 50%, below 40%, below 30%, below 20%, or below 10% that of a healthy control.

In some embodiments, a subject in need of treatment with a composition described herein can be identified by detecting low DNA, RNA, or protein levels associated with at least one endozepine biosynthesis enzyme (e.g., SEQ ID NO: 82284-90760 or 95264-95291) in the subject's stool (e.g., using whole-genome sequencing or gene-specific sequencing to detect the nucleic acids encoding the enzymes, or through proteomic analysis such as LC MS). In some embodiments, the level of at least one endozepine biosynthesis enzyme in the subject's stool is below about 80% that of a healthy control. In some embodiments, the level of at least one endozepine biosynthesis enzyme in the subject's stool is below 80%, below 70%, below 60%, below 50%, below 40%, below 30%, below 20%, or below 10% that of a healthy control.

Accordingly, the present disclosure provides for the treatment of endozepine associated mental illness or disease comprising administering to the subject one or more endozepine producing bacterial or yeast strains (e.g., purified strains) and/or their derivatives (e.g. live bacteria or yeast, dead bacteria or yeast, spent medium(s) derived from a bacteria or yeast, cell pellet(s) of a bacteria or yeast, purified metabolite(s) produced by bacteria or yeast, purified protein(s) produced by a bacteria or yeast, or combinations thereof), endozepines themselves, prebiotics (that stimulate the growth or activity of endozepine producing bacteria or yeast), and compositions comprising the same for administration to a subject in need thereof.

### Heavy Metal Sequestration

FIG. 1, Panel C illustrates a third exemplary CNS metabolic pathway targeted within the technology as described herein, defined herein as a “microbial-mediated heavy metal elimination”. The ability to excrete dietary heavy metals such as mercury depends on the composition and health of the gut microbiome. One mechanism for this dependence may involve microbial synthesis of siderophores, iron-scavenging molecules that also have the ability to sequester toxic heavy metals by forming insoluble complexes. Exemplifying this aspect, FIG. 1, Panel C depicts pydridine-2, 6-bis(thiocarboxylic acid) (PDTC), which can bind Cr, Pb, Hg, Cd, and As. Additionally, there is a wide variety of other microbial siderophores and other microbial products and processes implicated in microbiome-dependent heavy metal elimination, all of which are targets for substitution employing the methods and compositions described herein to treat cases of dysbiosis due to loss or impairment of heavy metal-eliminating microbial taxa, as described herein.

### Heavy Metal Sequestering Bacteria

In some embodiments the present disclosure provides one or more non-pathogenic heavy metal sequestering bacterial strains (e.g., purified strains) and/or their derivatives (e.g. live bacteria, dead bacteria, spent medium(s) derived from a bacteria, cell pellet(s) of a bacteria, purified metabolite(s) produced by bacteria, purified protein(s) produced by a bacteria, or combinations thereof) and compositions comprising the same for administration to a subject in need thereof. The bacteria can be naturally occurring, or can be engineered (e.g., through strain engineering or selection) to sequester heavy metal. In some embodiments, the bacteria sequester heavy metals via production of at least one siderophore. In some embodiments, one strain of siderophore producing bacteria can be administered to a subject in need thereof. In some embodiments, multiple strains of siderophore producing bacteria can be administered to a subject in need thereof. In some embodiments, the one or more bacteria (e.g., purified bacteria) can act synergistically. For instance, the multiple bacteria can act synergistically to produce high levels of at least one siderophore, via, including but not limited to, cross feeding of nutrients important for siderophore biosynthesis or via supporting growth or survival of siderophore producing bacteria. Additionally, the competition for a nutrient by a given bacterium (e.g. iron) can also elicit siderophore production in a siderophore producing bacteria. Accordingly, any one, or any combination of the siderophore producing bacteria described herein can be administered to a subject in need thereof.

In some embodiments, the heavy metal sequestering bacteria described herein can produce at least one siderophore under physiologically relevant conditions in the gut.

In some embodiments, a siderophore refers to a small peptidic molecule, readily assembled by short, dedicated metabolic pathways, which contain side chains and functional groups that can provide a high-affinity set of ligands for coordination of metals. In some embodiments, there are three main types of iron-coordinating functional groups in siderophores. First, there are the N-hydroxy amino acid side chains as in anguibactin, with the oxygen atom as one of the ligands for Fe3+. Second, there are the adjacent hydroxyls of catechol rings, almost always derived from 2,3-dihydroxybenzoate (DHB), as represented in enterobactin, anguibactin, and acinetobactin. Variants may involve biosynthetic use of 2-hydroxybenzoate (salicylate) in place of 2,3-DHB, leading to phenolic moieties as iron ligands. Third, the nitrogen atoms of five-membered thiazoline and oxazoline rings, resulting from enzymatic cyclization of cysteinyl, seryl, or threonyl side chains, respectively, can also bind to iron and other metals.

In some embodiments, the heavy metal sequestering bacteria described herein can produce at least one siderophore which binds to the heavy metal mercury or lead. In some embodiments, the siderophore binds preferentially to mercury or lead over other heavy metals, such as iron. As one example, bacterial produced siderophores with higher affinity to non-iron heavy metals are known for molybdenum, and generally siderophores have varying affinities for the metals they bind; see e.g., Liermann et al. Chemical Geology 220, 285-302 (2005).

In some embodiments, the heavy metal sequestering bacteria can be identified by having a 16S nucleic acid sequence with a substantial percent identity to a 16S sequence selected from SEQ ID NOs: 91399-91403, which have been found to possess genes encoding or directing the production of heavy-metal sequestering proteins or compositions (e.g., siderophore producing genes) encoded in their genomes. In some embodiments, the heavy metal sequestering bacteria can have at least 90% 16S sequence identity to a 16S sequence given in Table 2 (e.g., at least 91% identity, at least 92% identity, at least 93% identity, at least 94% identity, at least 95% identity, at least 96% identity, at least 97% identity, at least 98% identity, at least 99% identity, at least 99.5% identity, at least 99.9% identity, or 100% identity to one of SEQ ID NOs: 91399-91403).

In some embodiments, the heavy metal sequestering bacteria described herein can produce at least one siderophore that binds to mercury or lead, and that siderophore also does not support growth of known pathogens.

In some embodiments, the human gut siderophore producing bacteria are non-pathogenic bacteria belonging to a genus selected from the group consisting of: Azotobacter, Bacillus, Pantoea, and Rhizobium. In some embodiments, the human gut siderophore producing bacteria are non-pathogenic bacteria belonging to a species selected from the group consisting of: Azotobacter vinelandii, Bacillus megaterium, Bacillus subtilis, Pantoea allii, and Rhizobium radiobacter. In some embodiments, the human gut siderophore producing bacteria are non-pathogenic bacteria belonging to a strain selected from the group consisting of: Azotobacter vinelandii strain IAM 15004, Bacillus megaterium strain CT03, Bacillus subtilis strain IAM 12118, Pantoea allii strain BD 390, Rhizobium radiobacter (AM157353.1).

In some embodiments, the composition further comprises a pharmaceutically acceptable carrier, wherein the one or more isolated non-pathogenic heavy metal sequestering bacterial strains or an isolated product derived therefrom is present in an amount effective to alter heavy metal levels or bioavailable heavy metal levels in a subject in need thereof. Accordingly, in one aspect, described herein is a pharmaceutical composition comprising a siderophore, an analog, derivative or precursor thereof, or a combination of any of these, in an amount effective to alter heavy metal levels in a subject in need thereof, and a pharmaceutically acceptable carrier. In some embodiments, the siderophore, analog, derivative or precursor is isolated from a heavy metal sequestering bacterial strain or culture medium in which a queuine-producing bacterial or yeast strain has been cultured.

In some embodiments, a composition comprising one or more isolated, non-pathogenic heavy metal sequestering bacterial strains or an isolated product derived therefrom as described herein further comprises a different therapeutic composition in an amount effective to treat a CNS disease or disorder, non-limiting examples of which are described further herein.

In some embodiments, the heavy metal sequestering bacteria are identified by the presence of genes involved in siderophore biosynthesis (see e.g., Table 2), using genome sequencing, qPCR, or other related methods. In some embodiments, the siderophore biosynthesis genes are isochorismate pyruvate lyase (pchB; EC:4.2.99.21), salicylate biosynthesis/isochorismate synthase (pchA; EC 5.4.4.2), isochorismatase (entB; EC:3.3.2.1/6.3.2.14), 2,3-dihydro-2,3-dihydroxybenzoate dehydrogenase (entA; EC:1.3.1.28), or enterobactin synthetase component D (entD; EC:6.3.2.14 2.7.8.-). In some embodiments, the genes encoding enzymes involved in siderophore biosynthesis are of at least 50% amino acid sequence similarity with the representative sequences SEQ ID NOs 91407-95263 (e.g., at least 60% similarity, at least 70% similarity, at least 80% similarity, at least 90% similarity, at least 91% similarity, at least 92% similarity, at least 93% similarity, at least 94% similarity, at least 95% similarity, at least 96% similarity, at least 97% similarity, at least 98% similarity, at least 99% similarity, at least 99.5% similarity, at least 99.9% similarity, or 100% similarity). Enzymes produced by siderophore biosynthesis genes from other species of bacteria will catalyze the same reactions as those of the reference or representative enzymes.

In some embodiments, heavy metal sequestering composition producing bacteria are identified by growing isolated mammalian bacteria (e.g. human fecal bacteria) in multiple bacterial mediums (e.g. nutrient rich, nutrient poor, or environmentally similar to the mammalian gastrointestinal tract (e.g. similar pH or nutritional profiles)), and then measuring siderophore levels in the supernatants or cell pellets via LC/MS or other appropriate methods (e.g. the Blue Agar CAS Assay; see e.g., Louden et al., J Microbiol Biol Educ. 2011; 12(1): 51-53). In some embodiments, leveraging a collection of such identified siderophore producing bacteria, one can perform heavy metal binding competition assays to identify siderophores which bind to mercury or lead at a higher level than iron or other heavy metals. For example, one can mix an unpurified or purified siderophore (derived from siderophore producing bacteria) with an equal or varying concentration of a ferric substrate (e.g. ferric nitrate) and a heavy metal (e.g. mercury or lead). Incorporation of one metal over the other can be accessed via LC/MS or other methods appropriate for such detection; see e.g., Braud et al. J Bacteriol 191, 3517-3525, (2009). Such siderophores (or siderophore producing bacterial derivatives) can then be counter-screened against known pathogenic organisms known to use siderophores for virulence (e.g. Salmonella typhimurium), to identify siderophores with ideal binding affinities to target heavy metals (e.g. mercury or lead) but not able to support or enhance growth or virulence of a given or known pathogen.

In some embodiments, leveraging a collection of such identified siderophore producing bacteria and mediums in which they produce siderophores, one can furthermore identify prebiotics which further enhance siderophore production by these bacteria, by comparing levels of siderophore(s) in cultures with and without the candidate prebiotics. Similarly, one can employ such a method to identify synergistic combinations of siderophore producing bacteria (where the combination results in higher production of siderophore than the organisms alone).

In some embodiments, keystone siderophore producing bacteria are identified in the target mammalian species (e.g. a human) by mining or generating fecal, colonic wash, or intestinal biopsy transcriptome cohorts to identify which bacteria within a target mammal expresses genes involved in siderophore biosynthesis. Bacteria expressing genes involved in siderophore biosynthesis, particularly those bacteria found to have siderophores with high binding affinities to heavy metals, are strong candidates for the composition.

In some embodiments, genes involved in siderophore biosynthesis can be introduced into a host probiotic such as Escherichia coli Nissle 1917, where it expresses said genes to produce siderophore constitutively or inducibly. In some embodiments, point mutations or codon optimization in siderophore genes can be leveraged to change the binding site of siderophores, and consequently affinity for heavy metals.

In some embodiments, the compositions (including purified siderophores) can be administered as a drug, medical food, or nutraceutical.

In some embodiments, non-siderophore based mechanisms can also be leveraged to clear heavy metals. In a non-limiting example, compositions comprising bacteria which produce extracellular polymeric substances (EPS), or the EPS themselves, can be used to sequester heavy metal (see e.g., Francois et al. Appl Environ Microbiol 78, 1097-1106, (2012). Extracellular polymeric substances (EPSs) are natural polymers of high molecular weight secreted by microorganisms into their environment. EPSs establish the functional and structural integrity of biofilms, and are considered the fundamental component that determines the physiochemical properties of a biofilm. EPSs are mostly composed of polysaccharides (exopolysaccharides) and proteins, but include other macro-molecules such as DNA, lipids and humic substances. Bacteria that produce EPS can be identified by screening for mercury or lead tolerance in a panel of mammalian bacteria (e.g. human gut bacteria). Bacteria found to be tolerant to mercury or lead can then be tested for accumulation of mercury or lead in the cell pellets or supernatant. If the heavy metals are found in the supernatant, the bacteria secrete binding factors such as EPS to keep the mercury outside of the cell. If mercury is found in the cell, it is likely they sequester the mercury in vesicles away from other protein machinery. Genes involved in producing these heavy metal sequestering features can then be identified leveraging knockout strategies (e.g., CRISPR or transposon mutagenesis) or gain of function approaches.

In some embodiments, the heavy metal sequestering composition producing bacteria can be further profiled for suitability as a therapeutic, medical food, or nutraceutical by assessing the presence of wanted (e.g., fast growth rates, capability of surviving lyophilization at a recovery >0.1%, capable of growing in commercial manufacturing mediums) and unwanted (e.g., history of being a pathogen, antibiotic resistance to clinically relevant antibiotics, mobile elements, toxins, virulence factors, and a strong association with human disease) characteristics.

In some embodiments, the heavy metal sequestering related compositions can function in places beyond the brain, such as the gastrointestinal tract or the peripheral or enteric nervous systems. Without being limited by theory, this may be useful for conditions presenting with disrupted intestinal motility, pain, inflammation, or metabolic features.

### Heavy Metal Associated Diseases or Disorders

The heavy metal sequestering related compositions described herein can be administered to a patient in need thereof, for instance for the treatment of a mental illness or disease associated with high levels of mercury or lead (“heavy metal associated mental illness or disease”, also referred to herein as a central nervous system (CNS) disorder associated with heavy metals). Described herein are methods of use for such compositions.

In one aspect, described herein is a method of decreasing heavy metal levels in a subject in need thereof, the method comprising administering to the subject a composition as described herein in an amount effective to increase heavy metal levels in the subject. In some embodiments, the subject is a mammalian subject. In some embodiments, the subject is a human subject.

In one or more embodiments of any of the above-aspects, the heavy metal associated mental illness or disease that can be treated by administration of a composition described herein is selected from the group consisting of: depression, bipolar disorder, schizophrenia, anxiety, anxiety disorders, addiction, social phobia, major depressive disorder, treatment-resistant major depressive disorder (TR-MDD), major depressive disorder and its subtypes (melancholic depression, atypical depression, catatonic depression, postpartum depression, and seasonal affective disorder), Neurodegenerative amyloid disorders (Parkinson's, Alzheimer's, and Huntington's diseases) orthostatic tremor, Lafora disease, restless leg syndrome, neuropathic pain, pain disorders, dementia, epilepsy, stiff-person syndrome, premenstrual dysphoric disorder, autism spectrum disorder, sleep disorders, and attention deficit hyperactivity disorder (ADHD).

In some embodiments, the method further comprises decreasing at least one symptom of a heavy metal associated mental disorder or disease in the subject selected from the group consisting of: fatigue, insomnia, motor dysfunction, stress, persistent anxiety, persistent sadness, social withdrawal, substance withdrawal, irritability, thoughts of suicide, thoughts of self-harm, restlessness, low sex drive, lack of focus, loss of appetite, seizures, memory loss, anger, bouts of emotional reactivity, confusion, pain, and muscle spasms.

### Methods of Identifying Mammals in Need of Heavy Metal Sequestration

In some embodiments, the method of treatment can comprise first diagnosing a subject or patient who can benefit from treatment by a composition described herein. In some embodiments, the method further comprises administering to the patient a composition described herein.

In some embodiments, the process of identifying a subject with a heavy metal associated mental illness or disease can be carried out by a trained psychologist, psychiatrist, or neurologist. For instance, a psychiatrist, psychologist, or neurologist can diagnose a subject with a mental illness or disease of the central nervous system by evaluating the subject's behavior for symptoms of the mental illness or disease. One of skill in the art will understand that mental illness can also be identified in a subject with the aid of the Diagnostic and Statistical Manual of Mental Disorders (DSM-5), (American Psychiatric Association), or other relevant tools.

In one or more embodiments, the process of identifying a subject with a heavy metal associated mental illness or disease can comprise diagnosing the subject with a mental illness or disease. In some embodiments, the mental illness or disease is identified or diagnosed using fMRI. In some embodiments, mental illness or disease can be identified with standard psychological and neurological surveys, or in other methods known to experts in the field.

In some embodiments, a subject in need of treatment with a composition described herein can be identified by identifying high levels of heavy metals in the subject's blood, liver, brain, serum, stool, or other bodily fluid or tissue. In some embodiments, the amount of heavy metals in the subject's blood, liver, brain, serum, stool, or other bodily fluid or tissue (e.g., the initial amount of heavy metals in the subject's blood, liver, brain, serum, stool, or other bodily fluid or tissue) is above about 5.0 ug/mL gram or mL sample or tissue (e.g., as measured by LC/MS or other appropriate methods). In some embodiments, the amount of heavy metals in the subject's blood, liver, brain, serum, stool, or other bodily fluid or tissue (e.g., the initial amount of heavy metals in the subject's blood, liver, brain, serum, stool, or other bodily fluid or tissue) is above about 0.1 ug/mL, 0.5 ug/mL, 1.0 ug/mL, 2.5 ug/mL, 5.0 ug/mL, 10 ug/mL, 20 ug/mL, 50 ug/mL, or 100 ug/mL in the sample or tissue (e.g., as measured by LC/MS, proton magnetic resonance (PMR) or other appropriate methods).

In some embodiments, the percentage of heavy metal sequestering bacteria in the subject's gut (e.g., the initial amount) represents less than about 10% of total 16S rRNA sequences as measured by sequencing using such methods as 16S rRNA gene Illumina™ sequencing or quantitative PCR. In some embodiments, the percentage of heavy metal sequestering bacteria in the subject's gut represents about 9%, about 8%, about 7%, about 6%, about 5%, about 4%, about 3%, about 2%, about 1%, or less than about 1% of the total 16S sequences measured in the subject's gut.

In some embodiments of any of the above aspects, the amount of heavy metals in the subject's blood, liver, brain, serum, stool, or other bodily fluid or tissue is decreased 0.1%, 1%, 5%, 10%, 20%, 30%, 40%, 50%, 60%, 70%, 80%, 90%, 100%, 500%, 1000%, 2000%, 3000%, 4000%, 5000%, or more relative to the initial amount (e.g., as measured by LC/MS, proton magnetic resonance (PMR) or other appropriate methods). In some embodiments, at least one heavy metal sequestering bacteria is increased 0.1%, 1%, 5%, 10%, 20%, 30%, 40%, 50%, 60%, 70%, 80%, 90%, 100%, 500%, 1000%, 2000%, 3000%, 4000%, 5000%, or more in the subject's stool relative to the initial amount (e.g., as measured by qPCR, next generation sequencing, or other appropriate methods known to those familiar with the field). In some embodiments, the level of expression of at least one heavy metal sequestering enzyme (e.g. a siderophore producing gene) is increased 0.1%, 1%, 5%, 10%, 20%, 30%, 40%, 50%, 60%, 70%, 80%, 90%, 100%, 500%, 1000%, 2000%, 3000%, 4000%, 5000%, or more in the subject's sample relative to the initial level (e.g., as measured by qPCR, next generation sequencing, or other appropriate methods known to those familiar with the field).

In some embodiments, a subject in need of treatment with a composition described herein can be identified by detecting low levels of siderophore-producing bacteria (e.g., comprising 16S from SEQ ID NO: 91399-91403) in the subject's stool (e.g., using quantitative next-generation 16S sequencing). In some embodiments, the level of siderophore-producing bacteria in the subject's stool is below about 80% that of a healthy control. In some embodiments, the level of siderophore-producing bacteria in the subject's stool is below 80%, below 70%, below 60%, below 50%, below 40%, below 30%, below 20%, or below 10% that of a healthy control.

In some embodiments, a subject in need of treatment with a composition described herein can be identified by detecting low DNA, RNA, or protein levels associated with at least one siderophore biosynthesis enzyme (e.g., SEQ ID NO: 91407-95263) in the subject's stool (e.g., using whole-genome sequencing or gene-specific sequencing to detect the nucleic acids encoding the enzymes, or through proteomic analysis such as LC MS). In some embodiments, the level of at least one siderophore biosynthesis enzyme in the subject's stool is below about 80% that of a healthy control. In some embodiments, the level of at least one siderophore biosynthesis enzyme in the subject's stool is below 80%, below 70%, below 60%, below 50%, below 40%, below 30%, below 20%, or below 10% that of a healthy control.

Accordingly, the present disclosure provides for the treatment of heavy metal mental illness or disease comprising administering to the subject one or more siderophore producing bacterial strains (e.g., purified strains) and/or their derivatives (e.g. live bacteria, dead bacteria, spent medium(s) derived from a bacteria, cell pellet(s) of a bacteria, purified metabolite(s) produced by bacteria, purified protein(s) produced by a bacteria, or combinations thereof), purified siderophore, prebiotics, and compositions comprising the same for administration to a subject in need thereof.

### Treatment Methods

The compositions described herein can be administered to a patient in need thereof, for instance for the treatment of a queuine, endozepine and/or heavy metal-related disease or disorder. In some embodiments, the method of treatment can comprise first diagnosing a subject or patient who can benefit from treatment by a composition described herein. In some embodiments, such diagnosis comprises detecting or measuring a low level of queuine (or queuine precursor or queuine-associated metabolite) or a low level of endozepine (or endozepine precursor or endozepine-associated metabolite) or a low level of siderophore (or siderophore precursor or siderophore-associated metabolite) or a high heavy metal level in a sample from the subject or patient, each of which are examples of an abnormal level of each analyte. In other embodiments, such diagnosis comprises detecting or measuring low levels of queuine-producing, endozepine-producing, or heavy metal sequestering species in a sample, e.g., a sample of gut microbiota from the subject or patient, each of which are examples of an abnormal level of each analyte. In some embodiments, the method further comprises administering to the patient a composition described herein.

In some embodiments, the subject has previously been determined to have an abnormal level of an analyte described herein relative to a reference. In some embodiments, the reference level can be the level in a sample of similar cell type, sample type, sample processing, and/or obtained from a subject of similar age, sex and other demographic parameters as the sample/subject. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the step of determining if the subject has an abnormal level of an analyte described herein can comprise i) obtaining or having obtained a sample from the subject and ii) performing or having performed an assay on the sample obtained from the subject to determine/measure the level of the analyte in the subject. In some embodiments of any of the aspects, the step of determining if the subject has an abnormal level of an analyte described herein can comprise performing or having performed an assay on a sample obtained from the subject to determine/measure the level of analyte in the subject. In some embodiments of any of the aspects, the step of determining if the subject has an abnormal level of an analyte described herein can comprise ordering or requesting an assay on a sample obtained from the subject to determine/measure the level of the analyte in the subject. In some embodiments of any of the aspects, the step of determining if the subject has an abnormal level of an analyte described herein can comprise receiving the results of an assay on a sample obtained from the subject to determine/measure the level of the analyte in the subject. In some embodiments of any of the aspects, the step of determining if the subject has an abnormal level of an analyte described herein can comprise receiving a report, results, or other means of identifying the subject as a subject with a decreased level of the analyte.

In one aspect of any of the embodiments, described herein is a method of treating a queuine-associated, endozepine-associated, or heavy metal-associated disease or disorder in a subject in need thereof, the method comprising: a) determining if the subject has an abnormal level of an analyte described herein; and b) instructing or directing that the subject be administered a composition comprising at least one queuine, endozepine, or heavy metal modulating bacteria and/or product(s) produced thereby as described herein if the level of the analyte is decreased relative to a reference. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

### Administration

Compositions as described herein can be administered via any of a number of different routes or in different regimens. As used herein, the term “administering,” refers to the placement of a compound or bacteria or yeast as disclosed herein into a subject by a method or route which results in at least partial delivery of the agent at a desired site. Pharmaceutical or therapeutic compositions comprising the compounds or bacteria or yeast disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated. The period of viability of the bacteria or yeast cells after administration to a subject can be as short as a few hours, e.g., twenty-four hours, to a few days, to as long as several years, i.e., long-term engraftment.

In some embodiments, the methods described herein relate to treating a subject having or diagnosed as having a queuine, endozepine and/or heavy metal-related disease or disorder with a composition comprising at least one queuine, endozepine and/or heavy metal modulating bacteria or yeast and/or product(s) produced thereby as described herein. Subjects having a queuine, endozepine and/or heavy metal-related disease or disorder can be identified by a physician using current methods of diagnosing a queuine, endozepine and/or heavy metal-related disease or disorder. Symptoms and/or complications of a queuine, endozepine and/or heavy metal-related disease or disorder which characterize these conditions and aid in diagnosis are known in the art, as described above. Tests that can aid in a diagnosis of, e.g. a queuine, endozepine and/or heavy metal-related disease or disorder are described above and can include, in addition to standard measurements of queuine, endozepine and/or heavy metal itself, detection or measurement of gut bacteria or yeast that modulate queuine, endozepine and/or heavy metal, detection or measurement of genetic sequences of such bacteria or yeast, including 16S or 18S sequences and/or genetic sequences encoding proteins that modulate queuine, endozepine and/or heavy metal, or detection or measurement of bacterial or yeast metabolites or proteins that modulate queuine, endozepine and/or heavy metal. A family history of a queuine, endozepine and/or heavy metal-related disease or disorder, or exposure to risk factors for a queuine, endozepine and/or heavy metal-related disease or disorder can also aid in determining if a subject is likely to have a queuine, endozepine and/or heavy metal-related disease or disorder or in making a diagnosis of a queuine, endozepine and/or heavy metal-related disease or disorder.

In some embodiments, the methods described herein comprise administering an effective amount of a composition or compositions described herein, e.g. a composition comprising at least one queuine, endozepine and/or heavy metal modulating bacteria or yeast and/or product(s) produced thereby as described herein to a subject in order to alleviate a symptom of a queuine, endozepine and/or heavy metal-related disease or disorder. As used herein, “alleviating a symptom of a queuine, endozepine and/or heavy metal-related disease or disorder” is ameliorating any condition or symptom associated with the disease or disorder. As compared with an equivalent untreated control, such amelioration comprises a reduction by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral or parenteral administration. Administration can be local or systemic. It should be understood that administration routes will vary depending on the compositions being administered. For example, live bacteria or yeast, or even dead bacteria or yeast, will generally be administered via a route that delivers the composition to the gut, e.g., orally (including but not limited to orally with enteric delivery compositions), or rectally (e.g., via enema, colonoscope, or suppository), while purified polypeptides or metabolites can be delivered not only by these routes, but also, as appropriate, via ingestion, whether intravenous, subcutaneous, intraperitoneal, by inhalation, or by another parental route. The decisions for such delivery routes will be apparent to the ordinarily-skilled clinician.

The term “effective amount” as used herein refers to the amount of a composition comprising at least one queuine, endozepine and/or heavy metal modulating bacteria or yeast and/or product(s) produced thereby as described herein needed to alleviate at least one or more symptom of the disease or disorder, and relates to a sufficient amount of pharmacological composition to provide the desired effect. The term “therapeutically effective amount” therefore refers to an amount of a composition comprising at least one queuine, endozepine and/or heavy metal modulating bacteria or yeast and/or product(s) produced thereby as described herein that is sufficient to provide a particular anti-queuine, endozepine and/or heavy metal-related-disorder effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Microbes derived from the healthy human gut can generally be used over a wide range of doses without adverse effects. For products derived from, made from, or isolated from such microbes, effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, where a composition's active agent or ingredient comprises, consists essentially of, or consists of a metabolite or protein produced by a bacteria or yeast or bacterial or yeast composition as described herein, a dose can be formulated in animal models to achieve a concentration range in vivo that includes the IC50 (i.e., the concentration of a composition comprising at least one product of at least one queuine, endozepine and/or heavy metal modulating bacteria or yeast as described herein, which achieves a half-maximal inhibition of symptoms) as determined in cell culture, or in an appropriate animal model. Levels in biological samples can be measured, for example, by high performance liquid chromatography. The effects of any particular dosage can be monitored by a suitable bioassay, e.g., assay for queuine, endozepine and/or heavy metal, among others. The dosage can be determined by a physician and adjusted, as necessary, to suit observed effects of the treatment.

In some embodiments, the technology described herein relates to a pharmaceutical composition comprising a composition comprising at least one queuine, endozepine and/or heavy metal modulating bacteria or yeast and/or product(s) produced thereby as described herein, and optionally a pharmaceutically acceptable carrier. In some embodiments, the active ingredients of the pharmaceutical composition comprise at least one queuine, endozepine and/or heavy metal modulating bacteria or yeast and/or product(s) produced thereby as described herein. In some embodiments, the active ingredients of the pharmaceutical composition consist essentially of at least one queuine, endozepine and/or heavy metal modulating bacteria or yeast and/or product(s) produced thereby as described herein. In some embodiments, the active ingredients of the pharmaceutical composition consist of at least one queuine, endozepine and/or heavy metal modulating bacteria or yeast and/or product(s) produced thereby as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. at least one queuine, endozepine and/or heavy metal modulating bacteria or yeast and/or product(s) produced thereby as described herein.

In some embodiments, the composition comprising at least one queuine, endozepine and/or heavy metal modulating bacteria or yeast and/or product(s) produced thereby further comprises an enteric coating or similar composition to promote survival of or avoid the acidity of the stomach and permit delivery into the small or large intestines. Non-limiting examples of enteric coatings include cellulose acetate phthalate (CAP), hydroxypropyl methylcellulose phthalate (HPMCP), hydroxypropyl methylcellulose acetate succinate (HPMCAS), polyvinyl acetate phthalate, cellulose acetate trimellitate, shellac, polymethacrylic acid, polymethyl methacrylate, polyethyl methacrylate, polyethyl acrylate, hydroxypropyl methylcellulose, hydroxypropyl cellulose, polyvinylpyrrolidone, polyethylene glycol, polyvinyl alcohol, and mixtures thereof. In some embodiments, the enteric coating is pH sensitive. As a non-limiting example, the enteric coating dissolves at a pH greater than about 6.5-7, so as to prevent the release in the stomach and permit the release in the intestines. See e.g., US Patent Application 20190046457 and U.S. Pat. No. 9,486,487, the contents of each of which are incorporated herein by reference in their entireties.

In some embodiments, the pharmaceutical composition comprising a product derived from one or more queuine, endozepine and/or heavy metal modulating bacteria or yeast as described herein can be in a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a product derived from one or more queuine, endozepine and/or heavy metal modulating bacteria or yeast as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a queuine, endozepine and/or heavy metal modulating bacterial or yeast product as disclosed herein can also be incorporated into parenteral dosage, including conventional and controlled-release parenteral dosage forms.

Pharmaceutical compositions comprising at least one queuine, endozepine and/or heavy metal modulating bacteria or yeast and/or product(s) produced thereby can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions can contain a predetermined amount of a pharmaceutically acceptable salt of a bacterial or yeast-derived product, and can be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate release from the formulation. Depending on the pharmacology and pharmacokinetics of the composition, use of conventional dosage forms can lead to wide fluctuations in the concentrations of the composition in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control parameters such as a therapeutic composition's onset of action, duration of action, plasma levels within the therapeutic window, and peak blood levels. In particular, controlled- or extended-release dosage forms or formulations can be used to ensure that the maximum effectiveness of a therapeutic composition is achieved while minimizing potential adverse effects and safety concerns, which can occur both from under-dosing a therapeutic composition (i.e., going below the minimum therapeutic levels) as well as exceeding the toxicity level for the therapeutic composition. In some embodiments, the composition comprising at least one queuine, endozepine and/or heavy metal modulating bacteria or yeast and/or product(s) produced thereby as described herein can be administered in a sustained release formulation.

Controlled-release pharmaceutical products have a common goal of improving therapeutic composition therapy over that achieved by their non-controlled release counterparts. Ideally, the use of an optimally designed controlled-release preparation in medical treatment is characterized by a minimum of therapeutic composition substance being employed to cure or control the condition in a minimum amount of time. Advantages of controlled-release formulations include: 1) extended activity of the therapeutic composition; 2) reduced dosage frequency; 3) increased patient compliance; 4) usage of less total therapeutic composition; 5) reduction in local or systemic side effects; 6) minimization of therapeutic composition accumulation; 7) reduction in blood level fluctuations; 8) improvement in efficacy of treatment; 9) reduction of potentiation or loss of therapeutic composition activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of active ingredient that promptly produces the desired therapeutic effect, and gradually and continually release other amounts of such ingredient to maintain this level of therapeutic or prophylactic effect over an extended period of time. In order to maintain this constant level of active ingredient in the body, the ingredient must be released from the dosage form at a rate that will replace the amount of ingredient being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the compositions as described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the composition comprising at least one queuine, endozepine and/or heavy metal modulating bacteria or yeast and/or product(s) produced thereby as described herein described herein is administered as a monotherapy, e.g., another treatment for the queuine, endozepine and/or heavy metal-related disease or disorder is not administered to the subject.

In some embodiments of any of the aspects, the methods described herein can further comprise administering a second agent and/or treatment to the subject, e.g. as part of a therapy. The combination therapy, where employed, can be tailored to the particular indication. For example, where a queuine, endozepine and/or heavy metal-modulating bacteria or yeast or product(s) as described herein is administered to treat anxiety or depression, it can be administered in combination with an anti-anxiety or anti-depression therapeutic composition or therapy as known in the art or approved for clinical treatment of anxiety or depression. Other indications can be similarly treated with queuine, endozepine and/or heavy metal modulating bacteria or yeast or their products as described herein in combination with agents known in the art or approved for the clinical treatment of those indications.

Non-limiting examples of a second agent for treatment of cognitive disorders, mood disorders, anxiety disorders, psychiatric disorders, autism, bipolar disorder, major depression, anxiety and/or schizophrenia include: analgesic combinations, antimigraine agents, CGRP inhibitors, cox-2 inhibitors, miscellaneous analgesics, narcotic analgesic combinations, narcotic analgesics, Nonsteroidal anti-inflammatory drugs, salicylates, AMPA receptor antagonists, barbiturate anticonvulsants, benzodiazepine anticonvulsants, carbamate anticonvulsants, carbonic anhydrase inhibitor anticonvulsants, dibenzazepine anticonvulsants, fatty acid derivative anticonvulsants, gamma-aminobutyric acid analogs, gamma-aminobutyric acid reuptake inhibitors, hydantoin anticonvulsants, miscellaneous anticonvulsants, neuronal potassium channel openers, oxazolidinedione anticonvulsants, pyrrolidine anticonvulsants, succinimide anticonvulsants, triazine anticonvulsants, 5HT3 receptor antagonists, anticholinergic antiemetics, miscellaneous antiemetics, NK1 receptor antagonists, phenothiazine antiemetics, anticholinergic antiparkinson agents, dopaminergic antiparkinsonism agents, miscellaneous antiparkinson agents, barbiturates, benzodiazepines, miscellaneous anxiolytics, sedatives and hypnotics, cholinergic agonists, cholinesterase inhibitors, CNS stimulants, general anesthetics, muscle relaxants, VMAT2 inhibitors, lithium, or combinations thereof.

In certain embodiments, an effective dose of a composition comprising at least one queuine, endozepine and/or heavy metal modulating bacteria or yeast and/or product(s) produced thereby as described herein can be administered to a patient once. In certain embodiments, an effective dose of a composition comprising at least one queuine, endozepine and/or heavy metal modulating bacteria or yeast and/or product(s) produced thereby as described herein can be administered to a patient repeatedly. For systemic administration, subjects can be administered a therapeutic amount of a composition comprising for example a metabolite or product of a queuine, endozepine and/or heavy metal-modulating bacteria or yeast as described herein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Depending upon the indication, treatment according to the methods described herein can increase levels of a marker (e.g., queuine, endozepine and/or heavy metal or other marker) by at least 10%, at least 15%, at least 20%, at least 25%, at least 30%, at least 40%, at least 50%, at least 60%, at least 70%, at least 80% or at least 90% or more. Alternatively, treatment according to the methods described herein can reduce levels of a or symptom of a condition, e.g. a queuine, endozepine and/or heavy metal-related disease or disorder by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to a composition comprising at least one queuine, endozepine and/or heavy metal modulating bacteria or yeast and/or product(s) produced thereby as described herein. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. Alternative examples include dosing daily, every other day, twice weekly, every 10 days, every two weeks, once a month, every six weeks, every two months, or less frequently as required to maintain a beneficial effect. A composition comprising at least one queuine, endozepine and/or heavy metal modulating bacteria or yeast and/or product(s) produced thereby as described herein can be administered over a period of time, such as over a 5 minute, 10 minute, 15 minute, 20 minute, or 25 minute period.

The dosage ranges for the administration of a composition comprising at least one queuine, endozepine and/or heavy metal modulating bacteria or yeast and/or product(s) produced thereby as described herein, according to the methods described herein depend upon, for example, the form of the composition, its potency, and the extent to which symptoms, markers, or indicators of a condition described herein are desired to be reduced, for example the percentage reduction or increase desired for queuine, endozepine and/or heavy metal. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition comprising at least one queuine, endozepine and/or heavy metal modulating bacteria or yeast and/or product(s) produced thereby as described herein, e.g. the treatment of a condition described herein, or to induce a response as described herein (e.g. modulation of queuine, endozepine and/or heavy metal levels) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. For CNS disorders, such as depression or anxiety, among others, a change for the better by at least one increment on a clinically accepted scale of disease severity can be considered effective treatment. For example, an improvement on the Hamilton Depression Rating Scale, the Clinical Assessment of Depression (CAD), or other clinically-accepted scale of disease can indicate effective treatment. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal and includes: (1) inhibiting the disease, e.g., slowing or preventing a worsening of symptoms (e.g. depression, anxiety, etc.); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of queuine, endozepine and/or heavy metal-related disease or disorder. When using an experimental animal model, efficacy of treatment is evidenced when a statistically significant change in a marker is observed, e.g. queuine, endozepine and/or heavy metal.

The efficacy of a given dosage combination can also be assessed in an animal model, e.g. germ-free animal models or alternatively, in a specific pathogen-free (SPF) animal model, or in an animal model of a queuine, endozepine and/or heavy metal-related disease or disorder.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method for treating or preventing a gut microbiome dysbiosis-mediated central nervous system (CNS) disorder associated with queuine deficiency in a mammalian subject, comprising: administering to subjects dysbiotic for queuine producing gut microbes a probiotic containing an effective amount of a viable queuine producing bacterial strain capable of safely colonizing the subject's gut and viable and functional to re-establish normal microbiome queuine production levels in the gut, or a pharmaceutical composition comprising queuine in a dosage and delivery form suitable for queuine delivery to the gut and in an amount sufficient to meet or exceed normal gut queuine levels, whereby one or more symptoms of the CNS disorder associated with queuine deficiency in the subject is substantially alleviated.
2. The method of any one of the preceding paragraphs, wherein the CNS disorder is selected from a cognitive disorder, a mood disorder, an anxiety disorder, or a psychiatric disorder.
3. The method of any one of the preceding paragraphs, wherein the CNS disorder is selected from autism, bipolar disorder, major depression, anxiety or schizophrenia.
4. The method of any one of the preceding paragraphs, wherein the queuine producing bacterial strain is formulated for oral or mucosal delivery with a pharmaceutically acceptable excipient, carrier or diluent.
5. A nutritional supplement comprising: an isolated queuine producing mammalian gut-compatible bacterium, formulated and provided in sufficient bacterial numbers to colonize a gut of a mammalian subject following oral ingestion, said bacterium being viable and functional to support or establish normal microbiome queuine production levels in the gut.
6. A method for treating antibiotic-associated gut dysbiosis in a mammalian subject, comprising administering a nutritional supplement according to any one of the preceding paragraphs to the subject.
7. A feedstuff, food product, dietary supplement, nutritional supplement or food additive comprising: an isolated queuine producing mammalian gut-compatible bacterium, formulated and provided in sufficient bacterial numbers to colonize a gut of a mammalian subject following oral ingestion, said bacterium being viable and functional to support or establish normal microbiome queuine production levels in the gut.
8. A pharmaceutical composition comprising: an isolated queuine producing mammalian gut-compatible bacterium, formulated for oral or mucosal delivery and containing sufficient numbers of bacteria to colonize a gut of a mammalian subject following administration, the bacterium being viable and functional to support or establish normal microbiome queuine production levels in the gut after administration.
9. A pharmaceutical composition for treating a gut-dysbiosis associated CNS disorder in a mammalian subject exhibiting queuine and/or tetrahydrobiopterin deficiency comprising: queuine or a precursor or analog thereof in a dosage and delivery form suitable for delivery to the gut, in an amount sufficient to meet or exceed normal gut queuine levels, whereby one or more symptoms of the CNS disorder in the subject is substantially alleviated.

Some embodiments of the technology described herein can be defined according to any of the following numbered paragraphs:
1. A composition comprising one or more isolated, non-pathogenic queuine-producing bacterial strains or an isolated product derived therefrom.
2. The composition of paragraph 1, wherein the one or more isolated, non-pathogenic queuine-producing bacterial strains comprise live bacteria or dead bacteria, or wherein the isolated product derived therefrom comprises culture medium in which said one or more isolated, non-pathogenic bacterial strains have been cultured.
3. The composition of paragraph 1 or paragraph 2, wherein the isolated product derived therefrom comprises a purified polypeptide produced by the one or more bacterial strains.
4. The composition of any one of paragraphs 1-3, further comprising a pharmaceutically acceptable carrier, wherein the one or more isolated non-pathogenic queuine-producing bacterial strains or an isolated product derived therefrom is present in an amount effective to alter queuine levels in a subject in need thereof.
5. A pharmaceutical composition comprising queuine, an analog, derivative or precursor thereof, or a combination of any of these, in an amount effective to alter queuine levels in a subject in need thereof, and a pharmaceutically acceptable carrier.
6. The pharmaceutical composition of paragraph 5, wherein the queuine, analog, derivative or precursor is isolated from a queuine-producing bacterial strain or culture medium in which a queuine-producing bacterial strain has been cultured.
7. The composition of any one of paragraphs 1-6, wherein the at least one isolated non-pathogenic queuine producing bacteria is a human gut bacteria.
8. The composition of any one of paragraphs 1-7, wherein the at least one isolated non-pathogenic queuine-producing bacteria belongs to a species selected from Acetobacter pasteurianus, Achromobacter xylosoxidans, Acidaminococcus fermentans, Acidaminococcus intestini, Acinetobacter baumannii, Acinetobacter calcoaceticus, Acinetobacter junii, Acinetobacter lwoffii, Acinetobacter pittii, Acinetobacter radioresistens, Acinetobacter schindleri, Acinetobacter towneri, Acinetobacter ursingii, Acinetobacter variabilis, Adlercreutzia equolifaciens, Aeribacillus pallidus, Aeromonas caviae, Aeromonas enteropelogenes, Aeromonas hydrophila, Aeromonas jandaei, Aeromonas salmonicida, Aeromonas schubertii, Aeromonas veronii, Aggregatibacter aphrophilus, Akkermansia muciniphila, Alistipes onderdonkii, Alistipes putredinis, Allisonella histaminiformans, Anaeroglobus geminatus, Anaerostipes caccae, Anaerostipes hadrus, Aneurinibacillus aneurinilyticus, Aneurinibacillus migulanus, Anoxybacillus flavithermus, Asaccharobacter celatus, Bacillus altitudinis, Bacillus amyloliquefaciens, Bacillus aquimaris, Bacillus atrophaeus, Bacillus badius, Bacillus bataviensis, Bacillus cereus, Bacillus circulans, Bacillus clausii, Bacillus coagulans, Bacillus cohnii, Bacillus endophyticus, Bacillus firmus, Bacillus flexus, Bacillus fordii, Bacillus galactosidilyticus, Bacillus halodurans, Bacillus infantis, Bacillus koreensis, Bacillus kyonggiensis, Bacillus lentus, Bacillus licheniformis, Bacillus litoralis, Bacillus marisflavi, Bacillus megaterium, Bacillus mojavensis, Bacillus mycoides, Bacillus nealsonii, Bacillus okuhidensis, Bacillus pseudofirmus, Bacillus pseudomycoides, Bacillus pumilus, Bacillus simplex, Bacillus sonorensis, Bacillus subterraneus, Bacillus subtilis, Bacillus thuringiensis, Bacillus timonensis, Bacillus vallismortis, Bacillus vietnamensis, Bacillus weihenstephanensis, Bacteroides caccae, Bacteroides cellulosilyticus, Bacteroides clarus, Bacteroides coprocola, Bacteroides dorei, Bacteroides eggerthii, Bacteroides faecis, Bacteroides fragilis, Bacteroides intestinalis, Bacteroides massiliensis, Bacteroides nordii, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bacteroides xylanolyticus, Barnesiella intestinihominis, Barnesiella viscericola, Bilophila wadsworthia, Blautia luti, Bordetella bronchiseptica, Bordetella trematum, Brenneria alni, Brevibacillus agri, Brevibacillus brevis, Brevibacillus choshinensis, Brevibacillus formosus, Brevibacillus laterosporus, Brevibacillus parabrevis, Brevundimonas diminuta, Butyricimonas virosa, Campylobacter coli, Campylobacter concisus, Campylobacter curvus, Campylobacter gracilis, Campylobacter jejuni, Campylobacter showae, Campylobacter ureolyticus, Cedecea lapagei, Cedecea neteri, Chromohalobacter japonicus, Citrobacter amalonaticus, Citrobacter braakii, Citrobacter farmeri, Citrobacter freundii, Citrobacter gillenii, Citrobacter koseri, Citrobacter murliniae, Citrobacter youngae, Clostridium acetireducens, Clostridium bartlettii, Clostridium beijerinckii, Clostridium botulinum, Clostridium butyricum, Clostridium carboxidivorans, Clostridium colicanis, Clostridium diolis, Clostridium disporicum, Clostridium novyi, Clostridium ramosum, Clostridium sporogenes, Clostridium thermocellum, Coprococcus catus, Coprococcus eutactus, Cronobacter sakazakii, Delftia tsuruhatensis, Desulfovibrio desulfuricans, Desulfovibrio fairfieldensis, Desulfovibrio piger, Dialister invisus, Dialister pneumosintes, Enterobacter aerogenes, Enterobacter asburiae, Enterobacter cloacae, Enterobacter hormaechei, Enterobacter kobei, Enterobacter ludwigii, Enterorhabdus caecimuris, Erysipelatoclostridium ramosum, Escherichia coli, Escherichiafergusonii, Escherichia hermannii, Escherichia marmotae, Geobacillus stearothermophilus, Haemophilus influenzae, Haemophilus pittmaniae, Hafnia alvei, Halobacillus dabanensis, Halobacillus karajensis, Halobacillus salinus, Halobacillus trueperi, Helicobacter pylori, Intestinibacter bartlettii, Klebsiella oxytoca, Klebsiella pneumoniae, Klebsiella variicola, Kluyvera cryocrescens, Kluyvera georgiana, Kosakonia cowanii, Kushneria sinocarnis, Lachnospira pectinoschiza, Lachnotalea glycerini, Lactobacillus mali, Leclercia adecarboxylata, Lelliottia amnigena, Litorilituus sediminis, Lysinibacillus boronitolerans, Lysinibacillus fusiformis, Lysinibacillus massiliensis, Lysinibacillus sphaericus, Lysinibacillus xylanilyticus, Lysobacter soli, Megasphaera elsdenii, Megasphaera micronuciformis, Micrococcus lylae, Mitsuokella jalaludinii, Moellerella wisconsensis, Monoglobus pectinilyticus, Moraxella osloensis, Morganella morganii, Neisseria canis, Neisseria cinerea, Neisseria elongata, Neisseria flavescens, Neisseria gonorrhoeae, Neisseria macacae, Neisseria meningitidis, Neisseria mucosa, Neisseria perflava, Neisseria subflava, Nosocomiicoccus massiliensis, Noviherbaspirillum denitrificans, Oceanobacillus iheyensis, Oceanobacillus oncorhynchi, Oceanobacillus sojae, Ochrobactrum anthropi, Odoribacter splanchnicus, Oxalobacter formigenes, Paenibacillus alvei, Paenibacillus amylolyticus, Paenibacillus barcinonensis, Paenibacillus barengoltzii, Paenibacillus daejeonensis, Paenibacillus dendritiformis, Paenibacillus glucanolyticus, Paenibacillus illinoisensis, Paenibacillus lactis, Paenibacillus larvae, Paenibacillus lautus, Paenibacillus macerans, Paenibacillus naphthalenovorans, Paenibacillus odorifer, Paenibacillus pabuli, Paenibacillus pasadenensis, Paenibacillus polymyxa, Paenibacillus rhizosphaerae, Paenibacillus stellifer, Paenibacillus thiaminolyticus, Paenibacillus typhae, Pantoea agglomerans, Parabacteroides distasonis, Parabacteroides goldsteinii, Parabacteroides gordonii, Parabacteroides johnsonii, Parabacteroides merdae, Paraprevotella clara, Parasutterella excrementihominis, Peptoniphilus asaccharolyticus, Peptoniphilus indolicus, Planococcus rifietoensis, Porphyromonas asaccharolytica, Porphyromonas bennonis, Porphyromonas somerae, Prevotella bivia, Prevotella buccae, Prevotella buccalis, Prevotella copri, Prevotella timonensis, Proteus mirabilis, Proteus penneri, Proteus vulgaris, Providencia alcalifaciens, Providencia heimbachae, Providencia rettgeri, Providencia stuartii, Pseudomonas aeruginosa, Pseudomonas alcaligenes, Pseudomonas bauzanensis, Pseudomonas caricapapayae, Pseudomonas chlororaphis, Pseudomonas fluorescens, Pseudomonas fragi, Pseudomonas fulva, Pseudomonas gessardii, Pseudomonas japonica, Pseudomonas libanensis, Pseudomonas lundensis, Pseudomonas luteola, Pseudomonas migulae, Pseudomonas monteilii, Pseudomonas mosselii, Pseudomonas oleovorans, Pseudomonas oryzihabitans, Pseudomonas putida, Pseudomonas rhodesiae, Pseudomonas saudiphocaensis, Pseudomonas stutzeri, Pseudomonas taetrolens, Pseudomonas tolaasii, Pseudomonas xanthomarina, Psychrobacter phenylpyruvicus, Raoultella ornithinolytica, Raoultella planticola, Roseomonas gilardii, Roseomonas mucosa, Ruminococcus albus, Ruminococcus callidus, Ruminococcus flavefaciens, Ruminococcus lactaris, Ruminococcus torques, Salinisphaera halophila, Salinivibrio costicola, Salmonella enterica, Salmonella enteritidis, Salmonella typhi, Selenomonas ruminantium, Selenomonas sputigena, Senegalimassilia anaerobia, Serratia marcescens, Serratia ureilytica, Shewanella xiamenensis, Shigella boydii, Shigella dysenteriae, Shigella flexneri, Shigella sonnei, Sphingomonas aerolata, Staphylococcus arlettae, Staphylococcus aureus, Staphylococcus auricularis, Staphylococcus capitis, Staphylococcus caprae, Staphylococcus carnosus, Staphylococcus cohnii, Staphylococcus condimenti, Staphylococcus devriesei, Staphylococcus epidermidis, Staphylococcus equorum, Staphylococcus gallinarum, Staphylococcus haemolyticus, Staphylococcus hominis, Staphylococcus hyicus, Staphylococcus intermedius, Staphylococcus kloosii, Staphylococcus lentus, Staphylococcus lugdunensis, Staphylococcus nepalensis, Staphylococcus pasteuri, Staphylococcus petrasii, Staphylococcus pettenkoferi, Staphylococcus saccharolyticus, Staphylococcus saprophyticus, Staphylococcus schleiferi, Staphylococcus sciuri, Staphylococcus simiae, Staphylococcus simulans, Staphylococcus succinus, Staphylococcus vitulinus, Staphylococcus warneri, Staphylococcus xylosus, Stenotrophomonas acidaminiphila, Stenotrophomonas maltophilia, Stenotrophomonas rhizophila, Streptococcus australis, Streptococcus bovis, Streptococcus equinus, Streptococcus gallolyticus, Streptococcus infantarius, Streptococcus infantis, Streptococcus lutetiensis, Streptococcus mitis, Streptococcus mutans, Streptococcus oralis, Streptococcus peroris, Streptococcus pseudopneumoniae, Streptococcus salivarius, Streptococcus sobrinus, Streptococcus thermophilus, Streptococcus tigurinus, Streptococcus vestibularis, Succiniclasticum ruminis, Terribacillus aidingensis, Terribacillus halophilus, Thermotalea metallivorans, Turicibacter sanguinis, Veillonella atypica, Veillonella denticariosi, Veillonella dispar, Veillonella parvula, Vibrio cholerae, Victivallis vadensis, Virgibacillus massiliensis, Yersinia bercovieri, Yersinia enterocolitica, Yersinia intermedia, Yersinia kristensenii, Yersinia mollaretii, and combinations thereof.
9. The composition of any one of paragraphs 1-8, wherein the one or more non-pathogenic queuine producing bacteria is a human gut bacteria, and comprises a 16S rRNA sequence at least about 97% identical to a 16S rRNA sequence selected from SEQ ID NOs 1-406.
10. The composition of any one of paragraphs 1-9, wherein the at least one isolated non-pathogenic queuine producing bacteria is a human gut bacteria that encodes within its genome and expresses in the human gastrointestinal tract at least one queuine biosynthesis enzyme selected from folE (GTP cyclohydrolase), QueD (6-carboxy-5,6,7,8-tetrahydrobiopterin synthase), QueE (7-carboxy-7-deazaguanine synthase), QueC (7-cyano-7-deazaguanine synthase, PreQ0 synthase), QueF (7-cyano-7-deazaguanine reductase, PreQ0 reductase), tgt or btgt (tRNA guanine transglycosylase, bacterial tRNA guanine transglycosylase), QueA (S-adenosylmethionine:tRNA ribosyltransferase-isomerase), and QueG or QueH (epoxyqueuosine reductase).
11. The composition of any one of paragraphs 1-10, wherein the at least one isolated non-pathogenic queuine producing bacteria is a human gut bacteria that encodes within its genome and expresses in the human gastrointestinal tract at least one queuine biosynthesis enzyme, wherein the amino acid sequence encoded by the at least one queuine biosynthesis gene is at least 90% similar to a sequence selected from SEQ ID NOs 3660-82283.
12. The composition of any one of paragraphs 1-11, wherein the at least one isolated non-pathogenic queuine producing bacteria is a human gut bacteria belongs to species selected from Acidaminococcus fermentans, Adlercreutzia equolifaciens, Akkermansia muciniphila, Alloprevotella tannerae, Anaerostipes caccae, Anaerostipes hadrus, Arcobacter butzleri, Bacteroides caccae, Bacteroides cellulosilyticus, Bacteroides clarus, Bacteroides coprophilus, Bacteroides dorei, Bacteroides eggerthii, Bacteroides faecis, Bacteroides fragilis, Bacteroides massiliensis, Bacteroides nordii, Bacteroides oleiciplenus, Bacteroides ovatus, Bacteroides plebeius, Bacteroides salanitronis, Bacteroides salyersiae, Bacteroides stercoris, Bacteroides thetaiotaomicron, Bacteroides uniformis, Bacteroides vulgatus, Bacteroides xylanisolvens, Barnesiella intestinihominis, Bilophila wadsworthia, Butyrivibrio crossotus, Campylobacter curvus, Citrobacter freundii, Citrobacter koseri, Clostridium bartelettii, Clostridium ramosum, Coprobacter fastidiosus, Coprococcus catus, Coprococcus eutactus, Desulfovibrio piger, Dialister invisus, Dialister succinatiphilus, Enterobacter aerogenes, Enterobacter cancerogenus, Enterobacter cloacae, Enterorhabdus caecimuris, Escherichia coli, Eubacterium hallii, Fusobacterium mortiferum, Haemophilus pittmaniae, Haemophilus sputorum, Hafnia alvei, Klebsiella oxytoca, Klebsiella pneumoniae, Klebsiella variicola, Megamonas funiformis, Megamonas rupellensis, Megasphaera elsdenii, Megasphaera micronuciformis, Mitsuokella multacida, Odoribacter laneus, Odoribacter splanchnicus, Oxalobacter formigenes, Parabacteroides distasonis, Porphyromonas asaccharolytica, Porphyromonas uenonis, Ruminococcus callidus, Ruminococcus torques, Shigella sonnei, Streptococcus infantis, Streptococcus mitis, Streptococcus oralis, Streptococcus pneumoniae, Streptococcus tigurinus, Turicibacter sanguinis, Veillonella atypica, Veillonella dispar, Veillonella parvula, Dysgonomonas mossii, Proteus mirabilis, Veillonella ratti, and combinations thereof, and wherein the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.
13. The composition of any one of paragraphs 1-12, wherein the at least one isolated non-pathogenic queuine producing bacteria is a human gut bacteria with a 16S rRNA sequence at least about 97% identical to a 16S rRNA sequence selected from SEQ ID NOs 1-78, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.
14. The composition of paragraph 5 or 6, wherein the queuine precursor is epoxyqueuine and/or cobalamin.
15. The composition of paragraph 5 or 6, wherein the queuine analogs are selected from queuosine, a mannosyl queuosine, galactosyl queuosine, glutamyl queuosine, mannosylqueuine, galactosylqueuine, and aminoacylated derivatives such as glutamylqueuine.
16. The composition of any one of paragraphs 1-15, wherein the composition is formulated in a capsule, a tablet, a caplet, a pill, a troche, a lozenge, a powder, a granule, a nutraceutical, a medical food, or a combination thereof.
17. The composition of any one of paragraphs 1-16, formulated for delivery to the gut.
18. The composition of any one of paragraphs 1-17, further comprising a prebiotic.
19. The composition of any one of paragraphs 1-18, further comprising a different composition in an amount effective to treat a CNS disease or disorder.
20. The composition of any one of paragraphs 1-19, wherein the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or combinations thereof, and formulated for delivery with a pharmaceutically acceptable excipient, carrier or diluent.
21. A method of increasing queuine levels in a subject in need thereof, the method comprising administering to the subject a composition of any one of paragraphs 1-20 in an amount effective to increase queuine levels in the subject.
22. The method of paragraph 21, wherein the subject is a mammalian subject.
23. The method of paragraph 21 or 22, wherein the subject is a human subject.
24. A method for treating or preventing a gut microbiome dysbiosis-mediated central nervous system (CNS) disorder associated with queuine deficiency in a mammalian subject in need thereof, comprising administering to a subject dysbiotic for queuine producing gut microbes or low in queuine one or more isolated queuine-producing bacterial strains or an isolated product derived therefrom in an amount sufficient to increase queuine or to establish a queuine level within the range of normal in the subject, whereby one or more symptoms of the CNS disorder associated with queuine deficiency in the subject is improved.
25. A method for treating or preventing a central nervous system (CNS) disorder associated with queuine deficiency in a mammalian subject in need thereof, comprising administering to the subject a composition comprising an agent selected from queuine, a queuine precursor, or a queuine analog, in an amount sufficient to increase queuine or to establish a queuine level within the range of normal in the subject, whereby one or more symptoms of the CNS disorder associated with queuine deficiency in the subject is improved.
26. The method of paragraph 24 or 25, wherein the CNS disorder is selected from a cognitive disorder, a mood disorder, an anxiety disorder, and a psychiatric disorder.
27. The method of any one of paragraphs 24-27, wherein the CNS disorder is selected from autism, bipolar disorder, major depression, anxiety and schizophrenia.
28. The method of any one of paragraphs 21-27, further comprising identifying a subject in need of treatment by determining whether the subject would benefit from an increase in endogenous queuine.
29. The method of any one of paragraphs 21-28, wherein the amount of queuine in the subject's blood, liver, brain, serum, or stool is below 50 ng/mL.
30. The method of any one of paragraphs 21-29, wherein the amount of queuosine-modified Histidyl tRNA in a sample of the subject's blood, liver, brain, serum, or stool is less than 80% that of the total Histidyl tRNA in the sample.
31. The method of any one of paragraphs 21-30, wherein the amount of queuine-producing bacteria in the subject's stool is less than about 10% of total bacteria as measured by 16S sequence or shotgun sequencing.
32. The method of any one of paragraphs 21-31, wherein the amount of queuine, queuine-incorporated RNA, or BH4 in the subject's blood, liver, brain, serum, or stool is increased relative to the initial amount after administering the composition.
33. The method of any one of paragraphs 21-32, wherein the amount of queuine producing bacteria is increased in the subject's stool relative to the initial amount after administering the composition.
34. The method of any one of paragraphs 21-33, wherein the amount of queuine producing genes are increased in the subject's stool relative to the initial amount after administering the composition.
35. The method of any one of paragraphs 21-34, wherein the at least one isolated non-pathogenic queuine producing bacteria is a human gut bacteria, and belongs to the species selected from Acetobacter pasteurianus, Achromobacter xylosoxidans, Acidaminococcus fermentans, Acidaminococcus intestini, Acinetobacter baumannii, Acinetobacter calcoaceticus, Acinetobacter junii, Acinetobacter lwoffii, Acinetobacter pittii, Acinetobacter radioresistens, Acinetobacter schindleri, Acinetobacter towneri, Acinetobacter ursingii, Acinetobacter variabilis, Adlercreutzia equolifaciens, Aeribacillus pallidus, Aeromonas caviae, Aeromonas enteropelogenes, Aeromonas hydrophila, Aeromonas jandaei, Aeromonas salmonicida, Aeromonas schubertii, Aeromonas veronii, Aggregatibacter aphrophilus, Akkermansia muciniphila, Alistipes onderdonkii, Alistipes putredinis, Allisonella histaminiformans, Anaeroglobus geminatus, Anaerostipes caccae, Anaerostipes hadrus, Aneurinibacillus aneurinilyticus, Aneurinibacillus migulanus, Anoxybacillus flavithermus, Asaccharobacter celatus, Bacillus altitudinis, Bacillus amyloliquefaciens, Bacillus aquimaris, Bacillus atrophaeus, Bacillus badius, Bacillus bataviensis, Bacillus cereus, Bacillus circulans, Bacillus clausii, Bacillus coagulans, Bacillus cohnii, Bacillus endophyticus, Bacillus firmus, Bacillus flexus, Bacillus fordii, Bacillus galactosidilyticus, Bacillus halodurans, Bacillus infantis, Bacillus koreensis, Bacillus kyonggiensis, Bacillus lentus, Bacillus licheniformis, Bacillus litoralis, Bacillus marisflavi, Bacillus megaterium, Bacillus mojavensis, Bacillus mycoides, Bacillus nealsonii, Bacillus okuhidensis, Bacillus pseudofirmus, Bacillus pseudomycoides, Bacillus pumilus, Bacillus simplex, Bacillus sonorensis, Bacillus subterraneus, Bacillus subtilis, Bacillus thuringiensis, Bacillus timonensis, Bacillus vallismortis, Bacillus vietnamensis, Bacillus weihenstephanensis, Bacteroides caccae, Bacteroides cellulosilyticus, Bacteroides clarus, Bacteroides coprocola, Bacteroides dorei, Bacteroides eggerthii, Bacteroides faecis, Bacteroides fragilis, Bacteroides intestinalis, Bacteroides massiliensis, Bacteroides nordii, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bacteroides xylanolyticus, Barnesiella intestinihominis, Barnesiella viscericola, Bilophila wadsworthia, Blautia luti, Bordetella bronchiseptica, Bordetella trematum, Brenneria alni, Brevibacillus agri, Brevibacillus brevis, Brevibacillus choshinensis, Brevibacillus formosus, Brevibacillus laterosporus, Brevibacillus parabrevis, Brevundimonas diminuta, Butyricimonas virosa, Campylobacter coli, Campylobacter concisus, Campylobacter curvus, Campylobacter gracilis, Campylobacter jejuni, Campylobacter showae, Campylobacter ureolyticus, Cedecea lapagei, Cedecea neteri, Chromohalobacter japonicus, Citrobacter amalonaticus, Citrobacter braakii, Citrobacter farmeri, Citrobacter freundii, Citrobacter gillenii, Citrobacter koseri, Citrobacter murliniae, Citrobacter youngae, Clostridium acetireducens, Clostridium bartlettii, Clostridium beijerinckii, Clostridium botulinum, Clostridium butyricum, Clostridium carboxidivorans, Clostridium colicanis, Clostridium diolis, Clostridium disporicum, Clostridium novyi, Clostridium ramosum, Clostridium sporogenes, Clostridium thermocellum, Coprococcus catus, Coprococcus eutactus, Cronobacter sakazakii, Delftia tsuruhatensis, Desulfovibrio desulfuricans, Desulfovibrio fairfieldensis, Desulfovibrio piger, Dialister invisus, Dialister pneumosintes, Enterobacter aerogenes, Enterobacter asburiae, Enterobacter cloacae, Enterobacter hormaechei, Enterobacter kobei, Enterobacter ludwigii, Enterorhabdus caecimuris, Erysipelatoclostridium ramosum, Escherichia coli, Escherichiafergusonii, Escherichia hermannii, Escherichia marmotae, Geobacillus stearothermophilus, Haemophilus influenzae, Haemophilus pittmaniae, Hafnia alvei, Halobacillus dabanensis, Halobacillus karajensis, Halobacillus salinus, Halobacillus trueperi, Helicobacter pylori, Intestinibacter bartlettii, Klebsiella oxytoca, Klebsiella pneumoniae, Klebsiella variicola, Kluyvera cryocrescens, Kluyvera georgiana, Kosakonia cowanii, Kushneria sinocarnis, Lachnospira pectinoschiza, Lachnotalea glycerini, Lactobacillus mali, Leclercia adecarboxylata, Lelliottia amnigena, Litorilituus sediminis, Lysinibacillus boronitolerans, Lysinibacillus fusiformis, Lysinibacillus massiliensis, Lysinibacillus sphaericus, Lysinibacillus xylanilyticus, Lysobacter soli, Megasphaera elsdenii, Megasphaera micronuciformis, Micrococcus lylae, Mitsuokella jalaludinii, Moellerella wisconsensis, Monoglobus pectinilyticus, Moraxella osloensis, Morganella morganii, Neisseria canis, Neisseria cinerea, Neisseria elongata, Neisseria flavescens, Neisseria gonorrhoeae, Neisseria macacae, Neisseria meningitidis, Neisseria mucosa, Neisseria perflava, Neisseria subflava, Nosocomiicoccus massiliensis, Noviherbaspirillum denitrificans, Oceanobacillus iheyensis, Oceanobacillus oncorhynchi, Oceanobacillus sojae, Ochrobactrum anthropi, Odoribacter splanchnicus, Oxalobacter formigenes, Paenibacillus alvei, Paenibacillus amylolyticus, Paenibacillus barcinonensis, Paenibacillus barengoltzii, Paenibacillus daejeonensis, Paenibacillus dendritiformis, Paenibacillus glucanolyticus, Paenibacillus illinoisensis, Paenibacillus lactis, Paenibacillus larvae, Paenibacillus lautus, Paenibacillus macerans, Paenibacillus naphthalenovorans, Paenibacillus odorifer, Paenibacillus pabuli, Paenibacillus pasadenensis, Paenibacillus polymyxa, Paenibacillus rhizosphaerae, Paenibacillus stellifer, Paenibacillus thiaminolyticus, Paenibacillus typhae, Pantoea agglomerans, Parabacteroides distasonis, Parabacteroides goldsteinii, Parabacteroides gordonii, Parabacteroides johnsonii, Parabacteroides merdae, Paraprevotella clara, Parasutterella excrementihominis, Peptoniphilus asaccharolyticus, Peptoniphilus indolicus, Planococcus rifietoensis, Porphyromonas asaccharolytica, Porphyromonas bennonis, Porphyromonas somerae, Prevotella bivia, Prevotella buccae, Prevotella buccalis, Prevotella copri, Prevotella timonensis, Proteus mirabilis, Proteus penneri, Proteus vulgaris, Providencia alcalifaciens, Providencia heimbachae, Providencia rettgeri, Providencia stuartii, Pseudomonas aeruginosa, Pseudomonas alcaligenes, Pseudomonas bauzanensis, Pseudomonas caricapapayae, Pseudomonas chlororaphis, Pseudomonas fluorescens, Pseudomonas fragi, Pseudomonas fulva, Pseudomonas gessardii, Pseudomonas japonica, Pseudomonas libanensis, Pseudomonas lundensis, Pseudomonas luteola, Pseudomonas migulae, Pseudomonas monteilii, Pseudomonas mosselii, Pseudomonas oleovorans, Pseudomonas oryzihabitans, Pseudomonas putida, Pseudomonas rhodesiae, Pseudomonas saudiphocaensis, Pseudomonas stutzeri, Pseudomonas taetrolens, Pseudomonas tolaasii, Pseudomonas xanthomarina, Psychrobacter phenylpyruvicus, Raoultella ornithinolytica, Raoultella planticola, Roseomonas gilardii, Roseomonas mucosa, Ruminococcus albus, Ruminococcus callidus, Ruminococcus flavefaciens, Ruminococcus lactaris, Ruminococcus torques, Salinisphaera halophila, Salinivibrio costicola, Salmonella enterica, Salmonella enteritidis, Salmonella typhi, Selenomonas ruminantium, Selenomonas sputigena, Senegalimassilia anaerobia, Serratia marcescens, Serratia ureilytica, Shewanella xiamenensis, Shigella boydii, Shigella dysenteriae, Shigella flexneri, Shigella sonnei, Sphingomonas aerolata, Staphylococcus arlettae, Staphylococcus aureus, Staphylococcus auricularis, Staphylococcus capitis, Staphylococcus caprae, Staphylococcus carnosus, Staphylococcus cohnii, Staphylococcus condimenti, Staphylococcus devriesei, Staphylococcus epidermidis, Staphylococcus equorum, Staphylococcus gallinarum, Staphylococcus haemolyticus, Staphylococcus hominis, Staphylococcus hyicus, Staphylococcus intermedius, Staphylococcus kloosii, Staphylococcus lentus, Staphylococcus lugdunensis, Staphylococcus nepalensis, Staphylococcus pasteuri, Staphylococcus petrasii, Staphylococcus pettenkoferi, Staphylococcus saccharolyticus, Staphylococcus saprophyticus, Staphylococcus schleiferi, Staphylococcus sciuri, Staphylococcus simiae, Staphylococcus simulans, Staphylococcus succinus, Staphylococcus vitulinus, Staphylococcus warneri, Staphylococcus xylosus, Stenotrophomonas acidaminiphila, Stenotrophomonas maltophilia, Stenotrophomonas rhizophila, Streptococcus australis, Streptococcus bovis, Streptococcus equinus, Streptococcus gallolyticus, Streptococcus infantarius, Streptococcus infantis, Streptococcus lutetiensis, Streptococcus mitis, Streptococcus mutans, Streptococcus oralis, Streptococcus peroris, Streptococcus pseudopneumoniae, Streptococcus salivarius, Streptococcus sobrinus, Streptococcus thermophilus, Streptococcus tigurinus, Streptococcus vestibularis, Succiniclasticum ruminis, Terribacillus aidingensis, Terribacillus halophilus, Thermotalea metallivorans, Turicibacter sanguinis, Veillonella atypica, Veillonella denticariosi, Veillonella dispar, Veillonella parvula, Vibrio cholerae, Victivallis vadensis, Virgibacillus massiliensis, Yersinia bercovieri, Yersinia enterocolitica, Yersinia intermedia, Yersinia kristensenii, Yersinia mollaretii, and combinations thereof.
36. The method of any one of paragraphs 21-35, wherein the one or more non-pathogenic queuine producing bacteria is a human gut bacteria, and consists of one or more bacteria comprising a 16S rRNA sequence at least about 97% identical to a 16S rRNA sequence selected from SEQ ID NOs 1-406.
37. The method of any one of paragraphs 21-36, wherein the at least one isolated non-pathogenic queuine producing bacteria is a human gut bacteria that encodes within its genome and expresses in the human gastrointestinal tract at least one queuine biosynthesis selected from folE (GTP cyclohydrolase), QueD (6-carboxy-5,6,7,8-tetrahydrobiopterin synthase), QueE (7-carboxy-7-deazaguanine synthase), QueC (7-cyano-7-deazaguanine synthase, PreQ0 synthase), QueF (7-cyano-7-deazaguanine reductase, PreQ0 reductase), tgt or btgt (tRNA guanine transglycosylase, bacterial tRNA guanine transglycosylase), QueA (S-adenosylmethionine:tRNA ribosyltransferase-isomerase), and QueG or QueH (epoxyqueuosine reductase).
38. The method of any one of paragraphs 21-37, wherein the at least one isolated non-pathogenic queuine producing bacteria is a human gut bacteria that encodes within its genome and expresses in the human gastrointestinal tract at least one queuine biosynthesis enzyme, wherein the amino acid sequence encoded by the at least one queuine biosynthesis gene is at least 90% similar to a sequence selected from SEQ ID NOs 3660-82283.
39. The method of any one of paragraphs 21-38, wherein the at least one isolated non-pathogenic queuine producing bacteria is a human gut bacteria belongs to the species selected from Acidaminococcus fermentans, Adlercreutzia equolifaciens, Akkermansia muciniphila, Alloprevotella tannerae, Anaerostipes caccae, Anaerostipes hadrus, Arcobacter butzleri, Bacteroides caccae, Bacteroides cellulosilyticus, Bacteroides clarus, Bacteroides coprophilus, Bacteroides dorei, Bacteroides eggerthii, Bacteroides faecis, Bacteroides fragilis, Bacteroides massiliensis, Bacteroides nordii, Bacteroides oleiciplenus, Bacteroides ovatus, Bacteroides plebeius, Bacteroides salanitronis, Bacteroides salyersiae, Bacteroides stercoris, Bacteroides thetaiotaomicron, Bacteroides uniformis, Bacteroides vulgatus, Bacteroides xylanisolvens, Barnesiella intestinihominis, Bilophila wadsworthia, Butyrivibrio crossotus, Campylobacter curvus, Citrobacter freundii, Citrobacter koseri, Clostridium bartelettii, Clostridium ramosum, Coprobacter fastidiosus, Coprococcus catus, Coprococcus eutactus, Desulfovibrio piger, Dialister invisus, Dialister succinatiphilus, Enterobacter aerogenes, Enterobacter cancerogenus, Enterobacter cloacae, Enterorhabdus caecimuris, Escherichia coli, Eubacterium hallii, Fusobacterium mortiferum, Haemophilus pittmaniae, Haemophilus sputorum, Hafnia alvei, Klebsiella oxytoca, Klebsiella pneumoniae, Klebsiella variicola, Megamonas funiformis, Megamonas rupellensis, Megasphaera elsdenii, Megasphaera micronuciformis, Mitsuokella multacida, Odoribacter laneus, Odoribacter splanchnicus, Oxalobacter formigenes, Parabacteroides distasonis, Porphyromonas asaccharolytica, Porphyromonas uenonis, Ruminococcus callidus, Ruminococcus torques, Shigella sonnei, Streptococcus infantis, Streptococcus mitis, Streptococcus oralis, Streptococcus pneumoniae, Streptococcus tigurinus, Turicibacter sanguinis, Veillonella atypica, Veillonella dispar, Veillonella parvula, Dysgonomonas mossii, Proteus mirabilis, or Veillonella ratti, and combinations thereof, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.
40. The method of any one of paragraphs 21-39, wherein the at least one isolated non-pathogenic queuine producing bacteria is a human gut bacteria with a 16S rRNA sequence at least about 97% identical to a 16S rRNA sequence selected from SEQ ID NOs 1-78, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.
41. The method of paragraph 25, wherein the queuine precursors are selected from epoxyqueuine and/or cobalamin.
42. The method of paragraph 25, wherein the queuine analogs are selected from queuosine, a mannosyl queuosine, galactosyl queuosine, glutamyl queuosine, mannosylqueuine, galactosylqueuine, and aminoacylated derivatives such as glutamylqueuine.
43. The method of any one of paragraphs 21-42, wherein the composition is administered orally, intravenously, intramuscularly, intrathecally, subcutaneously, sublingually, buccally, rectally, vaginally, by the ocular route, by the otic route, nasally, via inhalation, by nebulization, cutaneously, transdermally, or combinations thereof, and formulated for delivery with a pharmaceutically acceptable excipient, carrier or diluent.
44. The method of any one of paragraphs 21-43, wherein the administered composition is formulated in a capsule, a tablet, a caplet, a pill, a troche, a lozenge, a powder, a granule, nutraceutical, a medical food, or a combination thereof.
45. The composition of any one of paragraphs 21-44, formulated for delivery to the gut.
46. The composition of any one of paragraphs 21-45, further comprising a prebiotic.
47. The composition of any one of paragraphs 21-46, further comprising a different composition in an amount effective to treat a CNS disease or disorder.
48. A composition comprising one or more isolated non-pathogenic endozepine-producing bacterial or yeast strains or an isolated product derived therefrom.
49. The composition of paragraph 48, wherein the one or more isolated, non-pathogenic endozepine-producing bacterial or yeast strains comprise live bacteria or yeast, or dead bacteria or yeast, or wherein the isolated product derived therefrom comprises culture medium in which said one or more isolated, non-pathogenic bacterial or yeast strains have been cultured.
50. The composition of paragraph 48 or 49, wherein the isolated product derived therefrom comprises a purified polypeptide produced by the one or more bacterial or yeast strains.
51. The composition of any one of paragraphs 48-50, further comprising a pharmaceutically acceptable carrier, wherein the one or more isolated non-pathogenic queuine-producing bacterial or yeast strains or an isolated product derived therefrom is present in an amount effective to alter endozepine levels in a subject in need thereof.
52. A pharmaceutical composition comprising endozepine, an analog, derivative or precursor thereof, or a combination of any of these, in an amount effective to alter endozepine levels in a subject in need thereof, and a pharmaceutically acceptable carrier.
53. The pharmaceutical composition of paragraph 52, wherein the endozepine analog, derivative or precursor is isolated from an endozepine-producing bacterial or yeast strain or culture medium in which an endozepine-producing bacterial or yeast strain has been cultured.
54. A method of increasing endozepine levels in a subject in need thereof, the method comprising administering to the subject a composition of any one of paragraphs 49-53 in an amount effective to increase endozepine levels in the subject.
55. The method of paragraph 54, wherein the subject is a mammalian subject.
56. The method of paragraph 54 or 55, wherein the subject is a human subject.
57. A method for treating or preventing a gut microbiome dysbiosis-mediated central nervous system (CNS) disorder associated with an endozepine deficiency in a mammalian subject in need thereof, comprising administering to a subject dysbiotic for endozepine producing gut microbes or low in endozepines one or more isolated non-pathogenic endozepine producing bacterial or yeast strains, an isolated product derived therefrom, endozepines, prebiotics, or combinations thereof, which alter endozepine levels in a subject in need thereof, wherein the composition is formulated for oral or intravenous delivery with a pharmaceutically acceptable excipient, carrier or diluent.
58. The method of paragraph 57, wherein the one or more isolated non-pathogenic endozepine producing bacterial or yeast strains comprises live bacteria or yeast, dead bacteria or yeast, spent medium(s) derived from a bacteria or yeast, cell pellet(s) of a bacteria or yeast, purified metabolite(s) produced by bacteria or yeast, purified protein(s) produced by a bacteria or yeast, and combinations thereof.
59. A composition comprising one or more isolated non-pathogenic heavy metal sequestering bacterial strains, their derivatives, siderophores, prebiotics, or combinations thereof, which alter heavy metal levels in a subject in need thereof, wherein the composition is formulated for oral or intravenous delivery with a pharmaceutically acceptable excipient, carrier or diluent.
60. The composition of paragraph 59, wherein the one or more isolated non-pathogenic heavy metal sequestering bacterial strains is a purified strain.
61. The composition of paragraph 59, wherein the one or more isolated non-pathogenic heavy metal sequestering bacterial strains comprises live bacteria, dead bacteria, spent medium(s) derived from a bacteria, cell pellet(s) of a bacteria, purified metabolite(s) produced by bacteria, purified protein(s) produced by a bacteria, and combinations thereof.
62. A method for treating or preventing a gut microbiome dysbiosis-mediated central nervous system (CNS) disorder associated with a heavy metal toxicity in a mammalian subject in need thereof, comprising administering to subjects dysbiotic for heavy metal sequestering gut microbes or high in toxic heavy metals one or more isolated non-pathogenic heavy metal sequestering bacterial strains (e.g., purified strains), their derivatives (e.g. live bacteria, dead bacteria, spent medium(s) derived from a bacteria, cell pellet(s) of a bacteria, purified metabolite(s) produced by bacteria, purified protein(s) produced by a bacteria, or combinations thereof), siderophores, prebiotics, or combinations thereof, which alter endozepine levels in a subject in need thereof, wherein the composition is formulated for oral or intravenous delivery with a pharmaceutically acceptable excipient, carrier or diluent.
63. The method of paragraph 62, wherein the one or more isolated non-pathogenic heavy metal sequestering bacterial strains is a purified strain.
64. The method of paragraph 62, wherein the one or more isolated non-pathogenic heavy metal sequestering bacterial strains comprises live bacteria, dead bacteria, spent medium(s) derived from a bacteria, cell pellet(s) of a bacteria, purified metabolite(s) produced by bacteria, purified protein(s) produced by a bacteria, and combinations thereof.
65. A method of increasing BH4 levels in a subject in need thereof, the method comprising administering to the subject a composition of any one of paragraphs 1-20 in an amount effective to increase BH4 levels in the subject.
66. The method of paragraph 65, wherein the subject is a mammalian subject.
67. The method of paragraph 65 or 66, wherein the subject is a human subject.
68. The composition of any one of paragraphs 1-20 and 45-47, for use in treating a queuine-related CNS disease or disorder.
69. The composition for use of paragraph 68, wherein the CNS disease or disorder is selected from a cognitive disorder, a mood disorder, an anxiety disorder, and a psychiatric disorder.
70. The composition for use of paragraph 68, wherein the CNS disorder is selected from autism, bipolar disorder, major depression, anxiety and schizophrenia.
71. The composition for use of any one of paragraphs 68-70, wherein treating comprises administering the composition to an individual diagnosed as having a queuine-related CNS disease or disorder.
72. The composition for use of any one of paragraphs 68-71, wherein treating comprises, prior to administering the composition for use, identifying a subject in need of treatment by determining whether the subject would benefit from an increase in endogenous queuine.
73. The composition for use of paragraph 72, wherein identifying a subject in need comprises measurement of the amount of queuine in the subject's blood, liver, brain, serum or stool.
74. The composition for use of any one of paragraphs 72 and 73, wherein identifying a subject in need comprises measurement of queuosine-modified Histidyl-tRNA in a sample of the subject's blood, liver, brain, serum or stool.
75. The composition for use of any one of paragraphs 72-74, wherein identifying a subject in need comprises measurement of queuine-producing bacteria in the subject's stool by 16S rRNA sequencing.
76. The composition for use of any one of paragraphs 72-75, wherein the amount of queuine-producing bacteria in the subject's stool is less than about 10% of total bacteria as measured by 16S rRNA sequencing.
77. Use of a composition of any one of paragraphs 1-20 and 45-47 for the treatment of a queuine-related CNS disease or disorder.
78. The use of paragraph 77, wherein the CNS disease or disorder is selected from a cognitive disorder, a mood disorder, an anxiety disorder, and a psychiatric disorder.
79. The use of paragraph 77, wherein the CNS disorder is selected from autism, bipolar disorder, major depression, anxiety and schizophrenia.
80. The composition of any one of paragraphs 1-20 and 45-47, for use in treating a gut microbial dysbiosis.
81. The composition for use of paragraph 80, wherein the gut microbial dysbiosis comprises a deficiency in queuine-producing gut bacteria.
82. The composition for use of any one of paragraphs 80-81, wherein treating comprises administering the composition to an individual diagnosed as having a deficiency in queuine-producing gut bacteria.
83. The composition for use of any one of paragraphs 80-82, wherein treating comprises, prior to administering the composition for use, identifying a subject in need of treatment by determining that the subject has a deficiency in queuine-producing gut bacteria.
84. The composition for use of paragraph 83, wherein identifying a subject in need comprises measurement of the amount of queuine in the subject's blood, liver, brain, serum or stool.
85. The composition for use of any one of paragraphs 83 and 84, wherein identifying a subject in need comprises measurement of queuosine-modified Histidyl-tRNA in a sample of the subject's blood, liver, brain, serum or stool.
86. The composition for use of any one of paragraphs 83-85, wherein identifying a subject in need comprises measurement of queuine-producing bacteria in the subject's stool by 16S rRNA sequencing.
87. The composition for use of any one of paragraphs 83-86, wherein the amount of queuine-producing bacteria in the subject's stool is less than about 10% of total bacteria as measured by 16S rRNA sequencing.
88. Use of a composition of any one of paragraphs 1-20 and 45-47, for treating a gut microbial dysbiosis.
89. The use of paragraph 88, wherein the gut microbial dysbiosis comprises a deficiency in queuine-producing gut bacteria.
90. The use of any one of paragraphs 88 and 89, wherein treating comprises administering the composition to an individual diagnosed as having a deficiency in queuine-producing gut bacteria.
91. The use of any one of paragraphs 88-90, wherein treating comprises, prior to administering the composition for use, identifying a subject in need of treatment by determining that the subject has a deficiency in queuine-producing gut bacteria.
92. The use of paragraph 91, wherein identifying a subject in need comprises measurement of the amount of queuine in the subject's blood, liver, brain, serum or stool.
93. The use of any one of paragraphs 91 and 92, wherein identifying a subject in need comprises measurement of queuosine-modified Histidyl-tRNA in a sample of the subject's blood, liver, brain, serum or stool.
94. The use of any one of paragraphs 91-93, wherein identifying a subject in need comprises measurement of queuine-producing bacteria in the subject's stool by 16S rRNA sequencing.
95. The use of any one of paragraphs 91-94, wherein the amount of queuine-producing bacteria in the subject's stool is less than about 10% of total bacteria as measured by 16S rRNA sequencing.
96. The composition of any one of paragraphs 1-20 and 45-47, for use in treating a BH4 deficiency or increasing the level of BH4 in a subject in need thereof.
97. Use of a composition of any one of paragraphs 1-20 and 45-47, for treating a BH4 deficiency or increasing the level of BH4 in a subject in need thereof.
98. The composition of any one of paragraphs 48-53, for use in treating or preventing a gut microbiome dysbiosis-mediated central nervous system (CNS) disorder associated with an endozepine deficiency in a mammalian subject in need thereof.
99. The composition for use of paragraph 98, wherein the CNS disease or disorder is selected from a cognitive disorder, a mood disorder, an anxiety disorder, and a psychiatric disorder.
100. The composition for use of paragraph 98, wherein the CNS disorder is selected from autism, bipolar disorder, major depression, anxiety and schizophrenia.
101. The composition for use of any one of paragraphs 98-100, wherein treating comprises administering the composition to an individual diagnosed as having a gut microbiome dysbiosis-mediated central nervous system (CNS) disorder associated with an endozepine deficiency.
102. The composition for use of any one of paragraphs 98-101, wherein treating comprises, prior to administering the composition for use, identifying a subject in need of treatment by determining whether the subject would benefit from an increase in endogenous endozepine.
103. The composition for use of paragraph 102, wherein identifying a subject in need comprises measurement of the amount of endozepine in the subject's blood, liver, brain, serum or stool.
104. The use of a composition of any one of paragraphs 48-53, for use in treating or preventing a gut microbiome dysbiosis-mediated central nervous system (CNS) disorder associated with an endozepine deficiency in a mammalian subject in need thereof.
105. The composition of any one of paragraphs 59-61, for use in treating or preventing a gut microbiome dysbiosis-mediated central nervous system (CNS) disorder associated with a heavy metal toxicity in a mammalian subject in need thereof.
106. The composition for use of paragraph 105, wherein treating comprises administering the composition to an individual diagnosed as having a gut microbiome dysbiosis-mediated central nervous system (CNS) disorder associated with a heavy metal toxicity.
107. The composition for use of any one of paragraphs 105-106, wherein treating comprises, prior to administering the composition for use, identifying a subject in need of treatment by determining whether the subject would benefit from a reduction in a heavy metal level.
108. Use of a composition of any one of paragraphs 59-61 for the treatment or prevention of a gut microbiome dysbiosis-mediated central nervous system (CNS) disorder associated with a heavy metal toxicity.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—the Gut Microbiome is Essential for Normal Queuine Levels in Mammals

In the instant example, the feasibility of acquiring sufficient queuine from diet alone was determined. Estimates were provided for queuine intake and depletion based on the model of Marks & Farkas (see e.g., Marks, T. & Farkas, W. R. Effects of a diet deficient in tyrosine and queuine on germfree mice. Biochem Biophys Res Commun 230, 233-237 (1997), the content of which is incorporated by reference herein in its entirety). Germ-free mice deprived of both tyrosine and queuine suffer serious adverse health effects, and die within a short period of time. Accordingly, the lack of tyrosine is fatal in this context, despite the presence of phenylalanine in the diet (which in principle the mice ought to be able to convert to tyrosine). Notably, introducing chemically synthesized queuine at a concentration of 0.1 μM to the diet prevented emergence of these deleterious effects, confirming that exogenous queuine is indispensable for production of sufficient amounts of tyrosine, and demonstrating that queuine supplementation according to the methods described herein can correct queuine-deficiency related adverse impacts on neurotransmitter synthetic components, mechanisms and pathways.

To further elucidate the nature and extent of mammalian dependency on microbiome-derived queuine, the germ-free mouse's daily dietary queuine requirement was calculated. Adult male Swiss Webster mice weigh about 40 g, and these subjects consume roughly 7 mL of liquid diet per day when fed ad lib. The molecular weight of queuine is 277.28 g/mol. Accordingly, 0.1 μM of queuine amounts to 27.7 ng/mL, as shown in formula (VII) below.

$$100 nMQ = \frac{27.7 μg Q}{1 L water} = 27.7 \frac{ng}{mL}$$

### (VII)

Factoring in the daily liquid consumption results in a calculated 193.9 ng queuine (Q) for the germ free mouse's daily dietary queuine requirement (upper bound), as shown in Formula (VIII), below.

$$\frac{27.7 n g Q}{1 mL} * \frac{7 mL liquid diet}{day} = \frac{193.9 n g Q}{day} = germfree \\ mouse ’ s daily dietary Q requirement ( upper bound ) \qquad ( VIII )$$

Factoring in the mouse's weight (0.04 kg), this amounts to approximately 0.00485 mg/kg per day, as shown in formula (IX) below.

$$\frac{194 \frac{n g Q}{day}}{0.04 kg} = \frac{0.00485 \frac{mg}{kg}}{day} = rough mammalian Q requirement ( upper bound ) \qquad ( IX )$$

Scaling this to an adult human, assuming 70 kg weight, the daily requirement of Q is roughly 0.34 mg queuine per day, as shown in formula (X) below.

$$\frac{0.00485 \frac{mg Q}{kg}}{day} * 70 kg = 0.339 \; 5 \frac{mg Q}{day} \approx 0.34 \frac{mg Q}{day} = rough human Q requiremen t ( upper bound ) \qquad ( X )$$

To illustrate dietary scenarios required to meet this queuine intake, three common foods with the highest concentrations of queuine (see, e.g., C. Fergus, et al., 2015, Nutrients 7, 2897-2929) were evaluated. Specifically, ripe coconut water has the highest known queuine concentration among common foods (87-530 ng/mL), with wheat germ in second place at 190 ng/g, and tomatoes in third at 21 ng/g. As such, to meet the predicted requirements of queuine the following intakes would be needed: 0.642 liters ripe coconut water per day (with a lower to upper bound of ˜3.9 L), 1.79 kg wheat germ per day, or 16.2 kg of tomatoes per day, as shown in formulas XI, XII, and XIII below.

$$\begin{matrix}\frac{0.3 \frac{mg Q}{day}}{530 \frac{n g}{mL}} = 0.6 \; 42 liters ripe coconut water / day & ( XI ) \\ \frac{0.34 \frac{mg Q}{day}}{190 ng / g} = 1.79 kg wheat germ per day & ( XII ) \\ \frac{0.34 \frac{mg Q}{day}}{21 \frac{n g}{g}} = 16.2 kg of tomatoes per day & \end{matrix}$$

### (XIII)

While these values represent an upper boundary, the relatively small concentrations of queuine in common foods indicates that dysbiosis involving queuine-producing gut microbes ordinarily causes serious queuine deficiency, which as shown herein can often be associated with adverse health impacts.

Based on these and other findings, the methods and compositions described herein effectively treat dysbiosis arising from impairment or loss of queuine-producing microbial species, by restoring queuine-positive microbes as a functional component of the microbiome in dysbiotic patients, and/or administering queuine directly (e.g., in a mucosal delivery form, such as an oral capsule, sublingual tablet, or suppository) to restore and maintain healthy queuine levels in the gut and through the essential compartments of the patient's body, including the CNS.

In more detailed aspects described herein, queuine-producing bacteria, or queuine compositions, are administered to dysbiotic mammalian subjects exhibiting queuine deficiency (e.g., as determined by screening for the presence of deficient queuine positive bacteria in the gut, or by measuring systemic queuine levels in the subject using blood or tissue samples) to treat or prevent a CNS disorder. In certain embodiments, treatment or prevention of a CNS disorder comprises modulating queuine levels in the subject, modulating tetrahydrobiopterin (BH4) levels in the subject, and/or modulating neurotransmitter levels in the subject.

Studies in germ-free animals further indicate that queuine deficiency can compromise CNS function, potentially through depletion of tetrahydrobiopterin (BH₄), a cofactor in the synthesis of crucial neurotransmitters (see e.g., Rakovich, et al. J. Biol. Chem 286, 19354-19363 (2011)). As described herein, a linked deficiency of queuine and BH4 in humans can often be associated with a CNS disorder mediated at least in part by gut microbiome dysbiosis effecting impairment or loss of microbial queuine production. The methods and compositions described herein, involving establishment or restoration of an effective population of queuine producing bacteria to the gut microbiome, or administration of queuine or BH4 to dysbiotic subjects sufficient to correct the queuine or tetrahydrobiopterine deficiency, are clinically effective to treat a range of associated CNS disorders, including but not limited to cognitive disorders exemplified by autism, mood disorders exemplified by bipolar disorder and major depression, anxiety disorders, and psychiatric disorders exemplified by schizophrenia.

The dependency of queuine depleted germ free mice on exogenous tyrosine occurs despite the presence of dietary phenylalanine, which can ordinarily be converted to tyrosine by the enzyme phenylalanine hydroxylase (PAH). Studies directed to a large population of schizophrenic patients, elucidate a role for queuine in this neurochemical synthetic process and functional pathway. In accordance with the technologies described herein, PAH exhausts BH4, which then depends on dihydropteridine reductase (DHPR) to recycle BH2 to BH4. This recycling is dependent at least in part on queuine, which likely plays a role in the synthesis of DHPR and possibly other antioxidants that normally prevent spontaneous oxidation of BH4. Among many prospective roles for queuine in these biosynthetic and recycling processes, there appears to be a requirement for queuosine tRNA modification for tyrosine biosynthesis by PAH.

### Example 2

### Queuine Deficiency and Disorders of Biopterin Regeneration

Tetrahydrobiopterin (BH4) Deficiency in Schizophrenic Human Patients

Schizophrenia is an extraordinarily debilitating mental health condition, which robs patients and their families of quality of life more severely, and with greater cost in terms of patient function, than most other mental health disorders. Over 200,000 schizophrenia cases are diagnosed in the US every year. While rare in toddlers and children, schizophrenia is common in all other age groups, from adolescents to seniors. Considerable attention in the scientific community has focused on a possible role of gut dysbiosis in schizophrenia, potentially mediating a neurochemical imbalance in schizophrenic subjects, but supporting evidence for such a connection is limited. Schizophrenic patients exhibit higher incidence and levels of Candida in their stool, which is directly associated with gut microbiome dysbiosis. However, a causal role for the dysbiosis is uncertain, given the fact that dysbiosis can arise from defective diet, intestinal disease such as cancer and inflammatory diseases, immune system dysfunction, and many other circumstances that might mediate dysbiosis and a co-occurring neurochemical imbalance causing clinical CNS disease development.

As described herein, gut microbial queuine deficiency is implicated as a direct causal factor in schizophrenia and other CNS disorders associated with neurochemical imbalance. Queuine function was assessed in 90 schizophrenic patients and 65 healthy controls, as determined by measuring blood tetrahydrobiopterin concentrations in these subjects (indicative of queuine-dependent BH2 to BH4 re-dox recycling efficiency); see e.g., Clelland et al. Schizophrenia research 210, 316-318, (2019). Blood samples were taken from the normal and schizophrenic patients and processed to preserve original BH2-BH4 ratios. The harvested samples were centrifuged to isolate a plasma fraction, and the plasma was transferred to sample containers containing the antioxidant dithioerythritol (DTE) (alternatively pentetic acid) to maintain the original activation state of biopterin. BH2 and BH4 levels in the patient and control samples were then assayed quantitatively using HPLC to determine differences between schizophrenic and normal subjects. These studies showed a statistically significant decrease in BH4 concentration in schizophrenic subjects compared to controls, indicating that queuine dependent redox recycling of BH2 to yield activated BH4 is substantially impaired and in clinical association with schizophrenia. Accordingly, these findings support that dysbiosis impairing or eliminating queuine producing gut microbes can mediate queuine deficiency, resulting in adverse impacts on neurochemical synthetic pathways associated with schizophrenia and other CNS disorders, including cognitive disorders, mood disorders, anxiety and other psychiatric disorders.

Further studies can be implemented using accepted animal models to quantify queuine deficiency in dysbiotic subjects. Without wishing to be bound by theory, it is expected that such subjects exhibit queuine deficiency-associated CNS disorder symptoms. In addition to demonstrating this direct linkage, these studies can demonstrate that the compositions and methods described herein involving therapeutic administration of queuine producing microbes (e.g., in an oral or mucosal probiotic formulation), or a pharmaceutical composition containing queuine itself, can correct neurochemical imbalances (e.g., restore normal BH2/BH4 levels essential for healthy neurotransmitter synthesis) and effectively treat or prevent symptoms of CNS disorders in dysbiotic patients and other subjects presenting with or at risk of gut microbial queuine depletion.

BH4-Associated Disorders

Tetrahydrobiopterin (BH₄) is essential for the function of aromatic amino acid hydroxylase (AAAH) enzymes, including tryptophan hydroxylase (TPH) and tyrosine hydroxylase (TH), in addition to PAH. To perform their respective hydroxylation reactions, each of these enzymes oxidizes BH₄ to a metastable form of BH₂, which must be enzymatically reduced before it can be reused. Examples of these neurochemical synthetic pathways are illustrated below in FIG. 2A-2B. While the technology described herein focuses on CNS ramifications of dysbiosis and queuine depletion, it is notable that all isoforms of nitric oxide synthase (NOS) also utilize BH₄ as a cofactor (see e.g., Crabtree et al. J Biol Chem 284, 28128-28136, (2009)). NOS generates nitric oxide, which is essential for proper function of the immune system, cardiovascular system, and nervous system. In this context, related aspects can effectively employ queuine producing microbes and queuine as a direct pharmaceutical agent or nutraceutical, to correct queuine-dependent BH4 deficiencies for the treatment and prevention of clinical conditions of the immune system, cardiovascular system, and nervous system associated with nitric oxide, NOS and/or BH4 quantitative or functional imbalances or deficiencies.

Correlated with the roles of queuine described herein, a limited availability of BH₄ in humans has other known pathological outcomes, as seen in genetic disorders that compromise BH₄ production or maintenance, which, in accordance with this disclosure, are expected to correlate with cognitive, emotional, and perceptual abnormalities. Given the importance of the AAAH enzymes in monoamine neurotransmitter synthesis (see e.g., FIG. 2A-2B), queuine dependent BH₄ deficit is expected to alter these synthetic pathways in a parallel manner as determined for schizophrenia, leading to mood, anxiety and psychiatric disorders associated with neurochemical dysfunction or deficit affecting each of these pathways. Targeting queuine deficiency in such disorders according to or using methods and/or compositions as described herein is therefore specifically contemplated for therapeutic benefit in disorders involving or characterized by BH4 deficiency.

FIG. 2A-2B illustrates how a queuine/tetrahydrobiopterin deficit can adversely influence multiple monoamine neurotransmitter systems. The dietary amino acids tryptophan, tyrosine, and phenylalanine are essential for the synthesis of important monoamine neurotransmitters, and key enzymes in these reactions utilize tetrahydrobiopterin (BH₄) as a cofactor, oxidizing it to dihydrobiopterin (BH₂) in the process. Regeneration of BH₄ requires the microbial metabolite queuine (see e.g., FIG. 1), and impaired BH₄ regeneration is expected to adversely impact monoamine neurotransmitter synthesis, enhancing production of atypical and potentially deleterious metabolites.

As illustrated in FIG. 2A, a shortage of usable BH₄ leads to a reduction in 5-HTP synthesis, and consequent depletion of serotonin and melatonin. Tryptophan is instead metabolized into kynurenic acid—a potential psychotomimetic associated with schizophrenia—and quinolinic acid, an excitotoxin. In FIG. 2B, a BH₄ shortage impairs the conversion of phenylalanine to tyrosine as well as the conversion of tyrosine to L-DOPA, reducing synthesis of the catecholamine neurotransmitters dopamine and norepinephrine. Unmetabolized phenylalanine is detrimental by itself, resulting in blockade of tryptophan and tyrosine transport into the brain. This enzymatic blockade also enhances the fraction of phenylalanine oxidized non-enzymatically into m-tyrosine (and also o-tyrosine, not shown), which can deplete brain concentrations of catecholamines as well. Other atypical metabolites of phenylalanine, such as phenethylamine, are also enhanced by this enzymatic blockade. In view of the above, it is clear that queuine deficiency can play a role in diseases or disorders involving or characterized by deficits in any or all of these monoamine neurotransmitters or monoamine neurotransmitter systems. Thus, targeting queuine deficiency in such disorders using methods and/or compositions as described herein is specifically contemplated for the treatment of such disorders.

Phenylalanine Hydroxylase, Phenylalanine, and Phenylketonuria

Other detailed aspects address gut dysbiosis mediated changes in metabolism and function of specific amino acid metabolism pathways in neurochemistry, and associated CNS disorders. Phenylalanine Hydroxylase (PAH) converts ingested phenylalanine into tyrosine, oxidizing BH₄ in the process. Without PAH, phenylalanine can build up to toxic concentrations in the body, a condition known as phenylketonuria. Symptoms of phenylketonuria may result from PHE's competitive inhibition of the large neutral amino acid transporter, which depletes tryptophan and tyrosine concentrations in the brain. In the absence of PAH, phenylalanine may be metabolized into atypical compounds such as m-tyrosine or phenethylamine, which can in turn impose complex deleterious effects on both the CNS and peripheral nervous systems (see e.g., Cleary, Paediatrics and Child Health 25, 108-112 (2015); Andersen & Avins, Arch Neurol 33, 684-686, (1976); Shaw Nutr Neurosci 13, 135-143, (2010); Dyck et al. European journal of pharmacology 84, 139-149, (1982)).

Regardless of the mechanism, neuropsychiatric symptoms of untreated phenylketonuria are well characterized, and can include paranoid ideation, anxiety, avolition, executive dysfunction, psychoticism, and a predisposition to seizures. Similar symptoms manifest to various degrees in depression, autism, schizophrenia, and other disorders. One study reported significantly elevated PHE levels in schizophrenic patients (see e.g., Okusaga et al. PLoS One 9, e85945, (2014)).

In view of the findings described herein relating to the clinical roles of queuine and BH4 in schizophrenia, it is also contemplated that the methods and compositions described herein can effectively prevent or alleviate gut dysbiosis-associated deficits in metabolism, activity and/or function of PAH and phenylalanine associated with phenylketonuria, and thereby effectively treat related CNS symptoms and disorders.

Tyrosine Hydroxylase, Tyrosine, and Catecholamine-Associated Disorders

In another detailed aspect, compositions and methods are provided to prevent or treat gut dysbiosis mediated changes in metabolism and function of tyrosine hydroxylase (TH) and tyrosine in the synthesis of catecholamine neurotransmitters dopamine and norepinephrine. In the CNS, TH oxidizes BH₄ to BH₂ as it performs its function of tyrosine conversion, and this BH4 recycling is a rate-limiting step in dopamine and norepinephrine synthesis. These neurotransmitters are essential for a variety of cognitive and emotional processes including alertness and attentive engagement, pleasure seeking, memory, and reward-prediction. As such, impaired activity of TH due to gut dysbiosis and attendant queuine and BH4 deficit, can often correlate with impaired dopamine and norepinephrine synthesis, and with associated adverse CNS impacts, for example anhedonia, lethargy, flat affect, attention deficit, and learning difficulties. All of the latter symptoms are associated with schizophrenia and, to some extent, depression and bipolar disorders, as well as attention deficit disorder. Dysfunction and death of dopaminergic neurons is also strongly implicated in Parkinson's disease, and a shortage of norepinephrine from the locus coeruleus has been proposed as a key mediator in the pathology of Alzheimer's disease (wherein norepinephrine promotes microglial phagocytosis of amyloid beta, and plays an additional role in curtailing neuroinflammation); see e.g., Heneka et al. PNAS 107, 6058-6063, (2010). In view of this, each of the disorders noted above is specifically contemplated for benefit from the methods and/or compositions described herein that increase or restore the production of queuine and/or queuine-related metabolites.

Tryptophan Hydroxylase, Tryptophan, and Serotonin or Melatonin-Associated Disorders

In yet additional detailed aspects, compositions and methods are provided to prevent or treat gut dysbiosis mediated changes in metabolism and function of Tryptophan Hydroxylase (TPH) and tryptophan in synthesis of the monoamine neurotransmitter serotonin. TPH is similar in structure and function to TH and PAH and likewise requires BH₄ to operate. As such, the technology described herein includes methods and compositions to prevent or alleviate serotonin deficiency and related CNS conditions associated with gut dysbiosis mediated queuine and BH4 deficiency. TPH also serves as a rate-limiting intermediate in the synthesis of serotonin, transforming dietary tryptophan into 5-HTP, which is thereafter decarboxylated to form serotonin (5-HT). Although serotonin's role in mood regulation is complex and incompletely understood, it certainly exerts powerful influences on emotion and cognition, whereby disruption of TPH's function through gut dysbiosis mediated queuine and/or BH4 deficiency is expected to adversely impact these and other CNS functions.

Serotonin deficiency is central to a classical “neurotransmitter imbalance” model of depression, which model is supported by modest efficacy of serotonergic pharmaceuticals to treat these conditions. A frequently overlooked function of serotonin in the brain is its role as the precursor to melatonin. A shortage of BH₄ significant enough to impact TPH activity would thereby subsequently impair melatonin synthesis and likely impair or reduce the quality of sleep. Disrupted sleep is associated with a multitude of negative outcomes across all physical, cognitive, and emotional scales, especially in memory consolidation and recall. Abnormalities in melatonin synthesis and sleep have been noted in schizophrenia, autism, depression, Alzheimer's disease, and Parkinson's disease. In view of this, each of the disorders noted above is specifically contemplated for benefit from the methods and/or compositions described herein that increase or restore the production of queuine and/or queuine-related metabolites.

As in the case of PHE and its atypical metabolites, tryptophan that is not converted (e.g., to produce serotonin) has the potential to be metabolized into a molecule of clinical significance, kynurenine. Kynurenine has two important metabolites for consideration herein, quinolinic acid (QUIN) and kynurenic acid (KYNA). QUIN is a compound with multiple demonstrated mechanisms of neurotoxicity, including excitotoxicity at the N-methyl-D-aspartate (NMDA) receptor, dysregulation of glutamatergic signaling, and the formation of reactive oxygen species in the presence of iron. Excess QUIN has been linked to major depressive disorder and suicidality (suicide attempters have more than double the ordinary concentration of QUIN in their cerebrospinal fluid); see e.g., Erhardt et al. Neuropsychopharmacology 38, 743-752, (2013). QUIN is formed spontaneously from the kynurenine pathway intermediate aminocarboxymuconate semialdehyde (ACMS) when activity of its associated decarboxylase enzyme (ACMSD) is insufficient to transform available ACMS into the neuroprotective metabolite picolinic acid. The ratio of quinolinic to picolinic acid has proven to be one of the strongest known predictors of suicidality; see e.g., Brundin et al. Transl Psychiatry 6, e865, (2016). Curiously, phthalate esters (plasticizing compounds that are ubiquitous as contaminants in food) inhibit the activity of ACMSD and enhance production of QUIN in vivo, suggesting a possible multifactorial interaction among diet, microbiome, genetics, and environmental toxin exposure in the etiology of suicidality.

Kynurenine can be transformed by kynurenine aminotransferase enzymes in the CNS to form KYNA, a compound that functions as an antagonist at the glycine site of the NMDA receptors. While it is often taken for granted that KYNA is neuroprotective (due to its ability to limit QUIN mediated neural damage), it should be noted that pharmacologically similar compounds, such as ketamine and phencyclidine (PCP), can induce delusions, hallucinations and sensations of depersonalization similar to symptoms seen in dissociative disorders, schizophrenia, and some cases of anxiety, depression, and bipolar disorders. Elevated concentrations of KYNA have been repeatedly observed in schizophrenic patients, which may account for this disorder's “positive” symptoms.

KYNA and other NMDA antagonists alter the firing patterns of dopaminergic neurons in the midbrain, increasing firing rate in the substantia nigra and ventral tegmental area. This alteration in dopaminergic activity may be a key to the psychotomimetic effects of NMDA antagonists, which are suppressed by the antipsychotic clozapine.

In view of the foregoing, gut dysbiosis mediated deficiencies of queuine and associated BH₄ dysregulation is considered herein to clinically predispose individuals to schizophrenia, in part attributable to a so-called “dopamine paradox” effect. Schizophrenic patients exhibit symptoms characteristic of both hyper- and hypo-activity of dopaminergic systems. Activity of TH may lead to disrupted learning and memory, and other symptoms typically associated with dopamine downregulation, while impaired activity of TPH may result in increased CNS production of KYNA, enhancing the activity of specific dopaminergic pathways in a manner that could be reversed by antipsychotic dopamine antagonists. While kynurenine readily crosses the blood-brain barrier, KYNA and QUIN do not. Additionally, these neurochemical players and pathways may explain the positive influence of physical fitness on psychiatric health. Exercise upregulates expression of peripheral kynurenine aminotransferases (via the protein PGC-1α), thereby enhancing conversion of kynurenine to KYNA before it can cross the blood-brain barrier and protecting against neurotoxic effects of QUIN and psychotomimetic effects of KYNA. In view of this, each of the disorders noted above is specifically contemplated for benefit from the methods and/or compositions described herein that increase or restore the production of queuine and/or queuine-related metabolites.

Treating Disorders of Biopterin Regeneration

The redox kinetics of biopterins and other cofactors involved in neurochemical synthesis and function are complex and sensitive to numerous uncertain factors. For example, exogenous BH₄ is rapidly oxidized to BH₂ before transport into the cell, after which it must be reduced before being put to use. As a result, administration of supplemental BH₄ has shown limited efficacy in treating disorders of biopterin regeneration. Furthermore, examining total biopterin concentrations, without differentiating between oxidation states, is unlikely to provide much insight where a given disorder is related to impairment in the regeneration of BH₄ from BH₂ (as disclosed here in the case of CNS disorders affected by queuine deficit). It should also be noted that the enzyme responsible for regenerating BH₂ to BH₄ (dihydropteridine reductase) can be inactivated by heavy metals (see e.g., Altindag et al. Toxicol In Vitro 17, 533-537, (2003)), providing a parallel pathway from dysbiosis to disorders of biopterin metabolism. Thus, disorders of biopterin regeneration resulting in or characterized by reduced levels of BH4 or an imbalance between BH2 and BH4 can be treated by administering a composition as described herein that promotes or increase queuine levels in the gut and/or a composition as described herein that promotes heavy metal sequestration.

In one embodiment, a subject can be treated with each of a composition that composition that promotes or increases queuine, a composition as described herein that promotes or increases endozepine levels, and a composition as described herein that promotes heavy metal sequestration.

Accordingly, additional aspects are directed to dysbiotic subjects that have complex, or multifactorial, gut biome dysbiosis—resulting in complex and often more severe clinical symptoms arising therefrom. For example, particularly severe dysbiosis can involve impairment or loss of multiple heirloom taxa expressing discrete, host-critical ex-genes. These cases result in impairment or loss of multiple distinct, important microbial functions, processes and/or products, and in many cases can be attended by multiple adverse clinical effects. In other cases, the subject gut taxa, ex-genes and related processes and products can be distinct between dysbiosis-impacted gut species, yet can be positively or negatively linked functionally, and interrelated clinically. For example, distinct processes and/or products of one dysbiotic gut species can ordinarily contribute (positively or negatively) to a common metabolic pathway or end-product as does a different microbial species, or otherwise exhibit “complementarity” in terms of biological activity or ultimate clinical effect(s). As used herein, “complementarity” is not limited to common activity, as might be expected to yield additive or synergistic biological effects and related clinical symptoms. Instead some complementary processes can involve attenuation or inhibition by one gut microbe negatively affecting expression or activity of processes and/or products of another gut microbe. In some cases, this form of complementarity can naturally serve to beneficially regulate processes and/or products of multiple microbes based on the presence, health and activity of another. In such cases, the impacts of severe dysbiosis can be even more critical, intractable and difficult to rectify or treat. Many examples likely exist of this type of functional complementarity between diverse gut microbes and their discrete processes and products, as there are complementary metabolic, signaling, developmental, neurochemical and other complex pathways regulating metabolism, cellular, tissue and organ function, homeostasis, CNS health and activity, and other critical functions of the mammalian body.

### Heavy Metal-Associated Disorders

In one exemplary embodiment relating to complex or multifactorial gut biome dysbiosis, subjects are effectively treated for a co-occurring gut microbiome-derived queuine deficiency, and simultaneous impairment of lead and/or mercury clearance attributable to a loss or impairment of gut microbes expressing processes and/or products involved in normal clearance of dietary heavy metals. Heavy metals like lead and mercury can inactivate critical enzymes, including DHPR, and thus can contribute to impairment of BH4 recycling. Individuals presenting with elevated lead and/or mercury, in combination with gut microbiome derived queuine deficiency, can present with more severe, interrelated adverse CNS symptoms, as described above.

CNS disorders arising from or exacerbated by impaired neurochemical synthesis attributable to a combination of queuine deficiency and elevated mercury or lead can be effectively treated or prevented using compositions and methods as described herein. In illustrative methods, these and other multifactorial cases of dysbiosis are treated using coordinate administration of a queuine producing bacteria, or a queuine pharmaceutical or supplement directly, and a viable, effective gut bacterium expressing one or more processes and/or products that direct(s), mediate(s) or facilitate(s) elimination and/or detoxification of a toxic heavy metal, such as dietary mercury or lead. Exemplary bacteria include all viable, non-pathogenic gut bacteria that produce siderophores (small molecules that bind and help eliminate heavy metals), PDTCs and other effectors that contribute to mercury or other heavy metal elimination (for example enterobactin, a high-affinity siderophore produced by Enterobacter sp).

Related aspects focus on gut microbiome dependent heavy metal elimination, and on the use of gut microbial probiotics and other compositions and methods to treat CNS disorders associated with elevated heavy metals concentration in the body. Heavy metals such as mercury (Hg) and lead are found in a wide variety of foods at trace concentrations, and can have severe deleterious effects on neurodevelopment and cognitive health. While these metals are generally bioaccumulative, the fraction of ingested heavy metals retained by the body and their subsequent impacts depend on numerous factors, including differential absorption and deposition of organic versus inorganic Hg.

The composition of a mammal's gut microbiome is one of many factors that affects its ability to excrete dietary Hg. Mice fed the highly neurotoxic compound methylmercury (MeHg) ordinarily retain only a fraction of the ingested dose. Six days after ingestion mice on three different defined diets were found to have excreted between 14% and 58% of the dose, depending on the diet. In mice pretreated with oral antibiotics, however, only 0-6% of the ingested dose was eliminated by day 6—almost all ingested Hg was retained in the body, regardless of diet; see e.g., Rowland et al. Arch Environ Health 39, 401-408, (1984). The means by which the microbiome mediates Hg elimination remains unclear, but Rowland et al.⁷⁹ suggest that microbial demethylation of MeHg may play a role by transforming the metal into inorganic varieties having reduced solubility in tissue.

Another mechanism relevant to metal elimination more broadly, involves microbial production of siderophores—molecules utilized by bacteria to scavenge iron from the extracellular environment. Many such molecules can trap metals other than iron—including cadmium, mercury, chromium, arsenic, and lead to form insoluble complexes. This sequestration protects both the microbe and its host from the deleterious effects of the metal, allowing it to be safely excreted. While the elimination half-life of Hg in humans is significantly different from that in rodents, recent research indicates that the underlying mechanisms are similarly dependent on the microbiome; see e.g., Rothenberg et al. Toxicol Lett 242, 60-67 (2016).

The relationship between antibiotic use and heavy metal retention can have health implications in domains ranging from agriculture to medicine. After a course of antibiotic treatment, the microbiome ideally returns to a near-baseline state to thereby restore microbial metabolic pathways and products that contribute to Hg elimination. However, in certain cases of dysbiosis it is expected that antibiotic-resistant species can outcompete more specialized taxa, e.g., by monopolizing nutrients, substrate and other resources in a manner that impairs or prevents regrowth by other more specialized, poorly competitive microorganisms. In extreme cases, this competitive suppression can greatly protract or even permanently impair a mammal's ability to eliminate dietary Hg and other heavy metals, resulting in associated profound impacts on CNS function and health.

Hg is one of the best studied and widely consumed heavy metals, occurring as methyl mercury (MeHg) in fish and other foods. MeHg is found at particularly high concentrations in predatory fish that exhibit bioaccumulation or biomagnification of certain toxins, but it is also found in many other foods and even drinking water. The presence of MeHg in the environment is due primarily to industrial waste emissions and fossil fuel pollution. Although it has multiple mechanisms of toxicity, MeHg can bind to the sulfhydryl groups present on thiol-containing amino acids. These are key functional groups in coordinating metal-enzyme complexes and ensuring proper protein folding throughout the body. As a result, MeHg interferes with a wide variety of processes including the transport and utilization of iron, zinc, copper, and selenium. The functions of these metals are numerous and diverse, but in general an imbalance or impairment of each of these metals can mediate profound effects on neurotransmitter metabolism, antioxidant enzyme activity, protection of cells from oxidative stress, and a variety of other important metabolic and homeostatic processes.

The nature and severity of CNS disorders caused by impaired Hg and other heavy metal detox/clearance vary depending on the dose, route, and rate of exposure. From human studies, acute mercury and other heavy metal toxicity imposes broad, adverse CNS effects, including impacts on depressive behaviors, loss of self-control, shyness, irritability, insomnia, and memory impairment, along with more characteristic neurological symptoms of metal intoxication including tremors, loss of sensation in the extremities, and impaired fine motor control. Altered metabolism of metals including Hg has been observed in a number of psychiatric and neurodegenerative disorders, including Alzheimer's disease and autism spectrum disorders (ASD), and some have hypothesized that dysregulation of metal dynamics has the potential to produce many of the symptoms associated with these conditions.

While much alarm has been falsely directed to fears that exposure to mercury in vaccines can cause autism or other CNS disorders, the minor exposure to mercury from certain vaccines has been ruled out as a causal factor in the development of autism and other CNS disorders. However, relatively little consideration has been paid to a more likely correlate of vaccine use, namely adverse impacts on the gut microbiome which might in some cases result in dysbiosis impairing mercury detoxification or elimination. There is a reported association between autism spectrum disorder (ASD) and dysbiosis (see e.g., De Angelis et al. Gut Microbes 6, 207-213, (2015)), and additional evidence points to elevated heavy metal concentrations in subjects with ASD. While ASD likely has a multifactorial etiology, with contributing factors including genetic predisposition and other environmental triggers, ASD patients are likely to suffer more severe CNS symptoms when they suffer gut dysbiosis resulting in impaired heavy metal metabolism. Consequently, the methods and compositions described herein that mediate restoration of gut microbes competent to assist in heavy detoxification and/or clearance, can substantially reduce symptoms and side effects of heavy metal toxicity in dysbiotic patients with autism and other CNS disorders.

### Endozepine-Associated Disorders

Additional methods and compositions are directed toward correcting gut dysbiosis mediated changes in endozepine synthesis important to normal CNS function and health. Benzodiazepines are anxiolytic drugs that act as positive allosteric modulators of gamma aminobutyric acid (GABA) receptor function, amplifying potency of the brain's primary inhibitory neurotransmitter by increasing ion flux through the GABA receptor's chloride channel, resulting in neuronal hyperpolarization and firing suppression. Impairment of GABAergic neurotransmission is observed in anxiety disorders and depression (see e.g., Kalueff & Nutt, Depress Anxiety 24, 495-517 (2007)), and this correlation underlies psychopharmacological models of anxiety disorders (positing that decreased activation of GABA receptors leads to an inability to quench activity in neural circuits related to somatic stress and worry, causing uncontrolled excitability and ruminative patterns of thought and behavior characteristic of anxiety and depression).

Long after benzodiazepine drugs were discovered, endogenous ligands of benzodiazepine receptors were identified in the mammalian brain, including one compound chemically identical to the drug diazepam (see e.g., Basile et al. N Engl J Med 325, 473-478 (1991)). This finding presented a paradox, because mammals lack the enzymatic machinery to perform the organochlorine chemistry capable of synthesizing diazepam and similar organochlorine compounds. In vivo investigations of this peculiarity led to the discovery that certain species of enteric microbe modulate CNS concentrations of small-molecule endozepines, likely via production of precursors that can be converted into an active form by the host animal; see e.g., Yurdaydin et al. Brain Res 679, 42-48 (1995). It has more recently been determined that certain microbial species in the human microbiome synthesize compounds comprising a core benzodiazepine structure, and/or express halogenase enzymes capable of mediating production of chlorinated organic molecules; see e.g., Roos, W. Ch. 2: Benzodiazepine Alkaloids, 63-97 (1990); Zehner et al. Chem Biol 12, 445-452 (2005). However, other compounds that do not share the classical 1,4-benzodiazepine structure can still have potent activity at the benzodiazepine receptor, and have been found in brain tissue of both humans and other mammals under physiologically relevant concentrations, including compounds with a quinoline core structure; see e.g., Rothstein et al. J Neurochem 58, 2102-2115 (1992). While many bacterial metabolites featuring the quinoline core structure are virulence factors or otherwise potentially harmful, some—such as pyrroloquinoline quinone (PQQ)—are harmless or even beneficial (see e.g., Goodwin & Anthony, Adv Microb Physiol 40, 1-80 (1998)), and their metabolites are prime candidates to play a role in modulating GABAergic activity in a healthy human body.

Research concerning endozepine-modulatory microbes has focused on a pathological excess of endozepines, as seen in hepatic encephalopathy, resulting in an endozepine model focused on the influence of liver function on GABAergic systems in the brain; see e.g., Basile et al. Neuropsychopharmacology 3, 61-71 (1990). However, another largely ignored facet is the potential for a pathological deficit of endozepines. Accordingly, gut dysbiosis that impairs or eliminates endozepine-modulatory microbes limits the host's obligate supply of endozepines, resulting in or contributing to CNS disorders that are treatable according to the methods and compositions described herein (e.g., by administering a probiotic comprising one or more viable non-pathogenic microbes expressing ex-genes, processes and/or products contributing to endozepine synthesis, metabolism and/or function).

Among the CNS disorders treatable using endozepine enhancing bacterial compositions and methods described herein, social anxiety and generalized anxiety disorder are both characterized by recurrent, intrusive, and pathologically intense psychological stress, disproportionate to any real threat posed by anxiogenic stimuli. On a temporary basis, exogenous benzodiazepines can be highly effective in relieving the symptoms of these disorders, although chronic administration can precipitate tolerance, dependence, and a rebound effect upon discontinuation. Considering the role of GABAergic neurotransmission in anxiety disorders, and the established but limited efficacy of benzodiazepines in their treatment, patients presenting with gut dysbiosis associated endozepine deficits and related CNS disorders, including anxiety disorders, can be effectively treated using the compositions and methods described herein that restore gut microbiome derived endozepines. Consistent with this aspect, some researchers have proposed a correlation between anxiety disorders and gastrointestinal disorders; see e.g., Walker et al. Am J Med 92, 26S-30S (1992). While it is possible that anxiety can result from other impacts of gut dysbiosis, the substitution of gut microbiome derived endozepines in dysbiotic patients is expected to yield clinical benefits in a majority of these cases.

### Example 3—Human Fecal Microbiome Signatures in Human Psychiatric Disease Show Reduced Levels of Queuine Producing Bacteria

Given the wealth of human microbiome sequencing data, an assessment of the bacterial composition of a select few humans' diseases relevant for this disclosure was performed.

For depression, there have been several reports which have identified lower levels of what are disclosed herein as keystone queuine producing bacteria. Specifically, a recent survey of 1,054 individuals identified that quality of life was correlated with the abundance of species of the genus Coprococcus and Dialister; see e.g., Valles-Colomer, M. et al. The neuroactive potential of the human gut microbiota in quality of life and depression. Nat Microbiol 4, 623-632, (2019), the content of which is incorporated herein by reference in its entirety. In two independent studies, it was similarly found that low levels of Dialister were associated with clinically depressed patients; see e.g., Kelly et al. J Psychiatr Res 82, 109-118 (2016); Jiang et al. Brain Behav Immun 48, 186-194 (2015). It is thus predicted herein that Dialister (e.g., SEQ ID NO: 40-41, Dialister invisus, Dialister succinatiphilus) and Coprococcus (e.g., SEQ ID NO: 0037-0038; e.g., Coprococcus catus or Coprococcus eutactus) are keystone human gut queuine producing bacteria. Conversely, in Kelly, 2016, higher levels of non-predicted queuine producers are reported in depressed patients (e.g., Eggerthella, Paraprevotella, Holdemania, and Gelria).

Similarly, for Schizophrenia, a recent cohort of 64 schizophrenia patients and 53 healthy controls found elevated levels of the genera Succinivibrio, Collinsella, Klebsiella and Methanobrevibacter but reduced levels of the genera Blautia and Coprococcus schizophrenic patients versus controls; see e.g., Shen et al. Schizophrenia research 197, 470-477 (2018). Succinivibrio, Collinsella, Klebsiella and Methanobrevibacter are not predicted to produce queuine in this disclosure, but Blautia (e.g., SEQ ID NO: 0154; e.g., Blautia luti) and Coprococcus (e.g., SEQ ID NO: 0037-0038; e.g., Coprococcus catus or Coprococcus eutactus) in this disclosure are predicted herein to be queuine producing bacteria. As described above, Coprococcus is also predicted herein to be a genus which actively expresses queuine producing machinery (“keystone queuine producing bacteria”).

These non-limiting examples provide support for the general concept that certain central nervous system disorders have a microbiome signature suggesting dysbiosis and a general reduction of keystone queuine producing bacteria. It can be presumed that such individuals would benefit from the compositions presented in this disclosure, which would correct disrupted queuine levels. Thus, in additional embodiments, provided herein are diagnostic methods in which the microbiome signature is evaluated, e.g., via 16S sequencing or transcriptional analysis for queuine-producing enzyme synthesis, among other approaches, to identify an individual as suffering from or at risk for one or more associated CNS disorders. In another embodiment, analysis of microbiome signature can be used to monitor the efficacy of a therapy for such associated CNS disorders using methods as described herein.

### References for Examples

1 Rosenberg, E. & Zilber-Rosenberg, I. Symbiosis and development: the hologenome concept. Birth Defects Res C Embryo Today 93, 56-66, doi:10.1002/bdrc.20196 (2011).
2 Salonen, A. & de Vos, W. M. Impact of diet on human intestinal microbiota and health. Annu Rev Food Sci Technol 5, 239-262, doi:10.1146/annurev-food-030212-182554 (2014).
3 Canani, R. B. et al. Potential beneficial effects of butyrate in intestinal and extraintestinal diseases. World J Gastroenterol 17, 1519-1528, doi:10.3748/wjg.v17.i12. 1519 10.3748/wjg.v17.i12.1519 (2011).
4 Hartstra, A. V., Bouter, K. E., Backhed, F. & Nieuwdorp, M. Insights into the role of the microbiome in obesity and type 2 diabetes. Diabetes Care 38, 159-165, doi:10.2337/dc14-0769 (2015).
5 Fergus, C., Barnes, D., Algasem, M. A. & Kelly, V. P. The queuine micronutrient: charting a course from microbe to man. Nutrients 7, 2897-2929, doi:10.3390/nu7042897 (2015).
6 Langgut, W., Reisser, T., Nishimura, S. & Kersten, H. Modulation of mammalian cell proliferation by a modified tRNA base of bacterial origin. FEBS letters 336, 137-142, doi:10.1016/0014-5793(93)81627-c (1993).
7 McCloskey, J. R. K. T. B. S. C. A. Why Is Tumor tRNA Hypomodified with Respect to Q Nucleoside. Modified Nucleosides and Cancer, 146-159 (1980).
8 Jacobson, K. B. Mechanism of suppression in Drosophila. VII. Correlation between disappearance of an isoacceptor of tyrosine tRNA and activation of the vermilion locus. Nucleic Acids Res 5, 2391-2404, doi:10.1093/nar/5.7.2391 (1978).
9 Emmanuel A. Meyer, N. D., Marine Guillot, W. Bernd Schweizer, François Diederich, Bernhard Stengl, Ruth Brenk, Klaus Reuter, and Gerhard Klebe. Synthesis, Biological Evaluation, and Crystallographic Studies of Extended Guanine-Based (lin-Benzoguanine) Inhibitors for tRNA-Guanine Transglycosylase (TGT). Helv. Chim. Acta 89, 573-597 (2006).
10 Katze, J. R., Basile, B. & McCloskey, J. A. Queuine, a modified base incorporated posttranscriptionally into eukaryotic transfer RNA: wide distribution in nature. Science 216, 55-56, doi:10.1126/science.7063869 (1982).
11 Bishara, A. et al. High-quality genome sequences of uncultured microbes by assembly of read clouds. Nat Biotechnol, doi:10.1038/nbt.4266 (2018).
12 Li, D., Liu, C. M., Luo, R., Sadakane, K. & Lam, T. W. MEGAHIT: an ultra-fast single-node solution for large and complex metagenomics assembly via succinct de Bruijn graph. Bioinformatics 31, 1674-1676, doi:10.1093/bioinformatics/btv033 (2015).
13 Bankevich, A. et al. SPAdes: a new genome assembly algorithm and its applications to single-cell sequencing. Journal of computational biology: a journal of computational molecular cell biology 19, 455-477, doi:10.1089/cmb.2012.0021 (2012).
14 Zhang, H., Cheng, Q. X., Liu, A. M., Zhao, G. P. & Wang, J. A Novel and Efficient Method for Bacteria Genome Editing Employing both CRISPR/Cas9 and an Antibiotic Resistance Cassette. Front Microbiol 8, 812, doi:10.3389/fmicb.2017.00812 (2017).
15 Murphy, K. C. Use of Bacteriophage λ Recombination Functions To Promote Gene Replacement in Escherichia coli. Journal of Bacteriology, 2063-2071 (1998).
16 Lagier, J. C. et al. Culturing the human microbiota and culturomics. Nat Rev Microbiol, 540-550, doi:10.1038/s41579-018-0041-0 (2018).
17 Matuszek, Z. a. P., T. Quantification of Queuosine Modification Levels in tRNA from Human Cells Using APB Gel and Northern Blot. Bio-protocol 9, doi:10.21769/BioProtoc.3191. (2019).
18 Yang, P., Yu, S., Cheng, L. & Ning, K. Meta-network: optimized species-species network analysis for microbial communities. BMC Genomics 20, 187, doi:10.1186/s12864-019-5471-1 (2019).
19 McGregor, K., Labbe, A. & Greenwood, C. M. T. MDiNE: A model to estimate differential co-occurrence networks in microbiome studies. Bioinformatics, doi:10.1093/bioinformatics/btz824 (2019).
20 Roth, J. R., Lawrence, J. G. & Bobik, T. A. Cobalamin (coenzyme B12): synthesis and biological significance. Annu Rev Microbiol 50, 137-181, doi:10.1146/annurev.micro.50.1.137 (1996).
21 Lawrence, J. G. & Roth, J. R. Evolution of coenzyme B12 synthesis among enteric bacteria: evidence for loss and reacquisition of a multigene complex. Genetics 142, 11-24 (1996).
22 Allen, R. H. & Stabler, S. P. Identification and quantitation of cobalamin and cobalamin analogues in human feces. Am J Clin Nutr 87, 1324-1335, doi:10.1093/ajcn/87.5.1324 (2008).
23 Johansson, T., Norris, T. & Peilot-Sjogren, H. Yellow fluorescent protein-based assay to measure GABA(A) channel activation and allosteric modulation in CHO-K1 cells. PLoS One 8, e59429, doi:10.1371/journal.pone.0059429 (2013).
24 Zimmermann, M., Zimmermann-Kogadeeva, M., Wegmann, R. & Goodman, A. L. Mapping human microbiome drug metabolism by gut bacteria and their genes. Nature 570, 462-467, doi:10.1038/s41586-019-1291-3 (2019).
25 Laura J. Liermann, R. L. G., Ariel Anbar, Susan L. Brantley. Production of a molybdophore during metal-targeted dissolution of silicates by soil bacteria. 220, 285-302 (2005).
26 Brian C. Louden, D. H., and Aaron M. Lynne. Use of Blue Agar CAS Assay for Siderophore Detection. 51-53 (2011).
27 Braud, A., Hannauer, M., Mislin, G. L. & Schalk, I. J. The Pseudomonas aeruginosa pyochelin-iron uptake pathway and its metal specificity. J Bacteriol 191, 3517-3525, doi:10.1128/JB.00010-09 (2009).
28 Francois, F. et al. Isolation and characterization of environmental bacteria capable of extracellular biosorption of mercury. Appl Environ Microbiol 78, 1097-1106, doi:10.1128/AEM.06522-11 (2012).
29 Marks, T. & Farkas, W. R. Effects of a diet deficient in tyrosine and queuine on germfree mice. Biochem Biophys Res Commun 230, 233-237, doi:10.1006/bbrc.1996.5768 (1997).
30 Tatsiana Rakovich, C. B., Ilana Bernstein, Vimbai M. Chikwana, Dirk Iwata-Reuyl, and Vincent P. Kelly. Queuosine Deficiency in Eukaryotes Compromises Tyrosine Production through Increased Tetrahydrobiopterin Oxidation. J. Biol. Chem 286, 19354-19363 (2011).
31 Clelland, J. D., Smeed, J., Cremers, S. & Clelland, C. L. A tetrahydrobiopterin deficit finding in schizophrenia: A confirmation study. Schizophrenia research 210, 316-318, doi:10.1016/j.schres.2019.06.006 (2019).
32 Thony, B., Auerbach, G. & Blau, N. Tetrahydrobiopterin biosynthesis, regeneration and functions. Biochem J 347 Pt 1, 1-16 (2000).
33 Crabtree, M. J., Tatham, A. L., Hale, A. B., Alp, N. J. & Channon, K. M. Critical role for tetrahydrobiopterin recycling by dihydrofolate reductase in regulation of endothelial nitric-oxide synthase coupling: relative importance of the de novo biopterin synthesis versus salvage pathways. J Biol Chem 284, 28128-28136, doi:10.1074/jbc.M109.041483 (2009).
34 Ponzone, A., Spada, M., Ferraris, S., Dianzani, I. & de Sanctis, L. Dihydropteridine reductase deficiency in man: from biology to treatment. Med Res Rev 24, 127-150, doi:10.1002/med.10055 (2004).
35 Cleary, M. A. Phenylketonuria. Paediatrics and Child Health 25, 108-112 (2015).
36 Andersen, A. E. & Avins, L. Lowering brain phenylalanine levels by giving other large neutral amino acids. A new experimental therapeutic approach to phenylketonuria. Arch Neurol 33, 684-686, doi:10.1001/archneur.1976.00500100018008 (1976).
37 Shaw, W. Increased urinary excretion of a 3-(3-hydroxyphenyl)-3-hydroxypropionic acid (HPHPA), an abnormal phenylalanine metabolite of Clostridia spp. in the gastrointestinal tract, in urine samples from patients with autism and schizophrenia. Nutr Neurosci 13, 135-143, doi:10.1179/147683010X12611460763968 (2010).
38 Dyck, L. E., Kazakoff, C. W. & Dourish, C. T. The role of catecholamines, 5-hydroxytryptamine and m-tyramine in the behavioural effects of m-tyrosine in the rat. European journal of pharmacology 84, 139-149, doi:10.1016/0014-2999(82)90196-0 (1982).
39 Bilder, D. A. et al. Psychiatric symptoms in adults with phenylketonuria. Mol Genet Metab 108, 155-160, doi:10.1016/j.ymgme.2012.12.006 (2013).
40 Rush, A. J., Gullion, C. M., Basco, M. R., Jarrett, R. B. & Trivedi, M. H. The Inventory of Depressive Symptomatology (IDS): psychometric properties. Psychol Med 26, 477-486, doi:10.1017/s0033291700035558 (1996).
41 Hill, E. L. Executive dysfunction in autism. Trends Cogn Sci 8, 26-32, doi:10.1016/j.tics.2003.11.003 (2004).
42 Chang Y T, C. P., Tsai I J, Sung F C, Chin Z N, Kuo H T, Tsai C H, Chou I C. Bidirectional relation between schizophrenia and epilepsy: a population-based retrospective cohort study. Epilepsia 52, 2036-2042, doi:10.1111/j.1528-1167.2011.03268.x (2011).
43 Tandon, R. et al. Definition and description of schizophrenia in the DSM-5. Schizophrenia research 150, 3-10, doi:10.1016/j.schres.2013.05.028 (2013).
44 Battle, D. E. Diagnostic and Statistical Manual of Mental Disorders (DSM). Codas 25, 191-192, doi:10.1590/s2317-17822013000200017 (2013).
45 Okusaga, O. et al. Elevated levels of plasma phenylalanine in schizophrenia: a guanosine triphosphate cyclohydrolase-1 metabolic pathway abnormality? PLoS One 9, e85945, doi:10.1371/journal.pone.0085945 (2014).
46 Naoi, M. & Maruyama, W. Cell death of dopamine neurons in aging and Parkinson's disease. Mech Ageing Dev 111, 175-188, doi:10.1016/s0047-6374(99)00064-0 (1999).
47 Heneka, M. T. et al. Locus ceruleus controls Alzheimer's disease pathology by modulating microglial functions through norepinephrine. Proc Natl Acad Sci USA 107, 6058-6063, doi:10.1073/pnas.0909586107 (2010).
48 Healy, D. Serotonin and depression. BMJ 350, h1771, doi:10.1136/bmj.h1771 (2015).
49 Jenkins, T. A., Nguyen, J. C., Polglaze, K. E. & Bertrand, P. P. Influence of Tryptophan and Serotonin on Mood and Cognition with a Possible Role of the Gut-Brain Axis. Nutrients 8, doi:10.3390/nu8010056 (2016).
50 Charney, D. S. Monoamine dysfunction and the pathophysiology and treatment of depression. The Journal of clinical psychiatry 59 Suppl 14, 11-14 (1998).
51 Yano, S., Moseley, K., Fu, X. & Azen, C. Evaluation of Tetrahydrobiopterin Therapy with Large Neutral Amino Acid Supplementation in Phenylketonuria: Effects on Potential Peripheral Biomarkers, Melatonin and Dopamine, for Brain Monoamine Neurotransmitters. PLoS One 11, e0160892, doi:10.1371/journal.pone.0160892 (2016).
52 Reilly, T. & Waterhouse, J. Altered sleep-wake cycles and food intake: the Ramadan model. Physiol Behav 90, 219-228, doi:10.1016/j.physbeh.2006.09.004 (2007).
53 Walker, M. P. & Stickgold, R. Sleep, memory, and plasticity. Annu Rev Psychol 57, 139-166, doi:10.1146/annurev.psych.56.091103.070307 (2006).
54 Anderson, G. & Maes, M. Melatonin: an overlooked factor in schizophrenia and in the inhibition of anti-psychotic side effects. Metab Brain Dis 27, 113-119, doi:10.1007/s11011-012-9307-9 (2012).
55 Melke, J. et al. Abnormal melatonin synthesis in autism spectrum disorders. Mol Psychiatry 13, 90-98, doi:10.1038/sj.mp.4002016 (2008).
56 Claustrat, B., Chazot, G., Brun, J., Jordan, D. & Sassolas, G. A chronobiological study of melatonin and cortisol secretion in depressed subjects: plasma melatonin, a biochemical marker in major depression. Biological psychiatry 19, 1215-1228 (1984).
57 Cardinali, D. P., Furio, A. M. & Brusco, L. I. Clinical aspects of melatonin intervention in Alzheimer's disease progression. Current neuropharmacology 8, 218-227, doi:10.2174/157015910792246209 (2010).
58 Medeiros, C. A. et al. Effect of exogenous melatonin on sleep and motor dysfunction in Parkinson's disease. A randomized, double blind, placebo-controlled study. J Neurol 254, 459-464, doi:10.1007/s00415-006-0390-x (2007).
59 Lugo-Huitron, R. et al. Quinolinic acid: an endogenous neurotoxin with multiple targets. Oxid Med Cell Longev 2013, 104024, doi:10.1155/2013/104024 (2013).
60 Erhardt, S. et al. Connecting inflammation with glutamate agonism in suicidality. Neuropsychopharmacology 38, 743-752, doi:10.1038/npp.2012.248 (2013).
61 Brundin, L. et al. An enzyme in the kynurenine pathway that governs vulnerability to suicidal behavior by regulating excitotoxicity and neuroinflammation. Transl Psychiatry 6, e865, doi:10.1038/tp.2016.133 (2016).
62 Schecter, A. et al. Phthalate concentrations and dietary exposure from food purchased in New York State. Environ Health Perspect 121, 473-494, doi:10.1289/ehp.1206367 (2013).
63 Fukuwatari, T., Ohsaki, S., Fukuoka, S., Sasaki, R. & Shibata, K. Phthalate esters enhance quinolinate production by inhibiting alpha-amino-beta-carboxymuconate-epsilon-semialdehyde decarboxylase (ACMSD), a key enzyme of the tryptophan pathway. Toxicol Sci 81, 302-308, doi:10.1093/toxsci/kfh204 (2004).
64 Erhardt, S., Schwieler, L., Nilsson, L., Linderholm, K. & Engberg, G. The kynurenic acid hypothesis of schizophrenia. Physiol Behav 92, 203-209, doi:10.1016/j.physbeh.2007.05.025 (2007).
65 Malhotra, A. K. et al. Ketamine-induced exacerbation of psychotic symptoms and cognitive impairment in neuroleptic-free schizophrenics. Neuropsychopharmacology 17, 141-150, doi:10.1016/S0893-133X(97)00036-5 (1997).
66 Linderholm, K. R. et al. Increased levels of kynurenine and kynurenic acid in the CSF of patients with schizophrenia. Schizophr Bull 38, 426-432, doi:10.1093/schbul/sbq086 (2012).
67 Szymona, K. et al. Correlations of Kynurenic Acid, 3-Hydroxykynurenine, sIL-2R, IFN-alpha, and IL-4 with Clinical Symptoms During Acute Relapse of Schizophrenia. Neurotox Res 32, 17-26, doi:10.1007/s12640-017-9714-0 (2017).
68 Erhardt, S., Oberg, H., Mathe, J. M. & Engberg, G. Pharmacological elevation of endogenous kynurenic acid levels activates nigral dopamine neurons. Amino Acids 20, 353-362, doi:10.1007/s007260170032 (2001).
69 Linderholm, K. R. et al. Activation of rat ventral tegmental area dopamine neurons by endogenous kynurenic acid: a pharmacological analysis. Neuropharmacology 53, 918-924, doi:10.1016/j.neuropharm.2007.09.003 (2007).
70 Malhotra, A. K. et al. Clozapine blunts N-methyl-D-aspartate antagonist-induced psychosis: a study with ketamine. Biological psychiatry 42, 664-668, doi:10.1016/s0006-3223(96)00546-x (1997).
71 Fukui, S., Schwarcz, R., Rapoport, S. I., Takada, Y. & Smith, Q. R. Blood-brain barrier transport of kynurenines: implications for brain synthesis and metabolism. J Neurochem 56, 2007-2017, doi:10.1111/j.1471-4159.1991.tb03460.x (1991).
72 Agudelo, L. Z. et al. Skeletal muscle PGC-1alpha1 modulates kynurenine metabolism and mediates resilience to stress-induced depression. Cell 159, 33-45, doi:10.1016/j.cell.2014.07.051 (2014).
73 Ohashi, A. et al. Tetrahydrobiopterin Supplementation: Elevation of Tissue Biopterin Levels Accompanied by a Relative Increase in Dihydrobiopterin in the Blood and the Role of Probenecid-Sensitive Uptake in Scavenging Dihydrobiopterin in the Liver and Kidney of Rats. PLoS One 11, e0164305, doi:10.1371/journal.pone.0164305 (2016).
74 Altindag, Z. Z., Baydar, T., Engin, A. B. & Sahin, G. Effects of the metals on dihydropteridine reductase activity. Toxicol In Vitro 17, 533-537, doi:10.1016/s0887-2333(03)00136-x (2003).
75 Hanninen, H. Behavioral effects of occupational exposure to mercury and lead. Acta neurologica Scandinavica. Supplementum 92, 167-175 (1982).
76 Bellinger, D., Leviton, A., Waternaux, C., Needleman, H. & Rabinowitz, M. Longitudinal analyses of prenatal and postnatal lead exposure and early cognitive development. N Engl J Med 316, 1037-1043, doi:10.1056/NEJM198704233161701 (1987).
77 Jedrychowski, W. et al. Effects of prenatal exposure to mercury on cognitive and psychomotor function in one-year-old infants: epidemiologic cohort study in Poland. Ann Epidemiol 16, 439-447, doi:10.1016/j.annepidem.2005.06.059 (2006).
78 Carrier, G., Brunet, R. C., Caza, M. & Bouchard, M. A toxicokinetic model for predicting the tissue distribution and elimination of organic and inorganic mercury following exposure to methyl mercury in animals and humans. I. Development and validation of the model using experimental data in rats. Toxicol Appl Pharmacol 171, 38-49, doi:10.1006/taap.2000.9112 (2001).
79 Rowland, I. R., Robinson, R. D. & Doherty, R. A. Effects of diet on mercury metabolism and excretion in mice given methylmercury: role of gut flora. Arch Environ Health 39, 401-408, doi:10.1080/00039896.1984.10545872 (1984).
80 Miethke, M. & Marahiel, M. A. Siderophore-based iron acquisition and pathogen control. Microbiol Mol Biol Rev 71, 413-451, doi:10.1128/MMBR.00012-07 (2007).
81 Zawadzka, A. M., Crawford, R. L. & Paszczynski, A. J. Pyridine-2,6-bis(thiocarboxylic acid) produced by Pseudomonas stutzeri KC reduces chromium(VI) and precipitates mercury, cadmium, lead and arsenic. Biometals 20, 145-158, doi:10.1007/s10534-006-9022-2 (2007).
82 Schalk, I. J., Hannauer, M. & Braud, A. New roles for bacterial siderophores in metal transport and tolerance. Environ Microbiol 13, 2844-2854, doi:10.1111/j.1462-2920.2011.02556.x (2011).
83 Jo, S. et al. Estimation of the Biological Half-Life of Methylmercury Using a Population Toxicokinetic Model. Int J Environ Res Public Health 12, 9054-9067, doi:10.3390/ijerph120809054 (2015).
84 Rothenberg, S. E. et al. The role of gut microbiota in fetal methylmercury exposure: Insights from a pilot study. Toxicol Lett 242, 60-67, doi:10.1016/j.toxlet.2015.11.022 (2016).
85 Zahir, F., Rizwi, S. J., Haq, S. K. & Khan, R. H. Low dose mercury toxicity and human health. Environ Toxicol Pharmacol 20, 351-360, doi:10.1016/j.etap.2005.03.007 (2005).
86 Joensuu, O. I. Fossil fuels as a source of mercury pollution. Science 172, 1027-1028, doi:10.1126/science.172.3987.1027 (1971).
87 Kumar, C. et al. Glutathione revisited: a vital function in iron metabolism and ancillary role in thiol-redox control. EMBO J 30, 2044-2056, doi:10.1038/emboj.2011.105 (2011).
88 Chmielnicka, J., Brzeznicka, E. & Sniady, A. Kidney concentrations and urinary excretion of mercury, zinc and copper following the administration of mercuric chloride and sodium selenite to rats. Archives of toxicology 59, 16-20, doi:10.1007/bf00263951 (1986).
89 Bernhoft, R. A. Mercury toxicity and treatment: a review of the literature. J Environ Public Health 2012, 460508, doi:10.1155/2012/460508 (2012).
90 in World Health Organization.
91 Adams, J. B., Romdalvik, J., Ramanujam, V. M. & Legator, M. S. Mercury, lead, and zinc in baby teeth of children with autism versus controls. J Toxicol Environ Health A 70, 1046-1051, doi:10.1080/15287390601172080 (2007).
92 Arora, M. et al. Fetal and postnatal metal dysregulation in autism. Nature communications 8, 15493, doi:10.1038/ncomms15493 (2017).
93 Mutter J, C. A., Naumann J, Deth R, Walach H. Does inorganic mercury play a role in Alzheimer's disease? A systematic review and an integrated molecular mechanism. Journal of Alzheimer's disease: JAD 22, 357-374, doi:10.3233/JAD-2010-100705. (2010).
94 Parker, S. K., Schwartz, B., Todd, J. & Pickering, L. K. Thimerosal-containing vaccines and autistic spectrum disorder: a critical review of published original data. Pediatrics 114, 793-804, doi:10.1542/peds.2004-0434 (2004).
95 De Angelis, M., Francavilla, R., Piccolo, M., De Giacomo, A. & Gobbetti, M. Autism spectrum disorders and intestinal microbiota. Gut Microbes 6, 207-213, doi:10.1080/19490976.2015.1035855 (2015).
96 Nemeroff, C. B. The role of GABA in the pathophysiology and treatment of anxiety disorders. Psychopharmacol Bull 37, 133-146 (2003).
97 Kalueff, A. V. & Nutt, D. J. Role of GABA in anxiety and depression. Depress Anxiety 24, 495-517, doi:10.1002/da.20262 (2007).
98 Basile, A. S. et al. Elevated brain concentrations of 1,4-benzodiazepines in fulminant hepatic failure. N Engl J Med 325, 473-478, doi:10.1056/NEJM199108153250705 (1991).
99 Yurdaydin, C. et al. Gut bacteria provide precursors of benzodiazepine receptor ligands in a rat model of hepatic encephalopathy. Brain Res 679, 42-48, doi:10.1016/0006-8993(95)00241-h (1995).
100 Roos, W. Ch. 2: Benzodiazepine Alkaloids. 63-97 (1990).
101 Zehner, S. et al. A regioselective tryptophan 5-halogenase is involved in pyrroindomycin biosynthesis in Streptomyces rugosporus LL-42D005. Chem Biol 12, 445-452, doi:10.1016/j.chembiol.2005.02.005 (2005).
102 Rothstein, J. D. et al. Purification and characterization of naturally occurring benzodiazepine receptor ligands in rat and human brain. J Neurochem 58, 2102-2115, doi:10.1111/j.1471-4159.1992.tb10952.x (1992).
103 Goodwin, P. M. & Anthony, C. The biochemistry, physiology and genetics of PQQ and PQQ-containing enzymes. Adv Microb Physiol 40, 1-80, doi:10.1016/s0065-2911(08)60129-0 (1998).
104 Basile, A. S., Ostrowski, N. L., Gammal, S. H., Jones, E. A. & Skolnick, P. The GABAA receptor complex in hepatic encephalopathy. Autoradiographic evidence for the presence of elevated levels of a benzodiazepine receptor ligand. Neuropsychopharmacology 3, 61-71 (1990).
105 Davidson, J. R. T. Use of benzodiazepines in social anxiety disorder, generalized anxiety disorder, and posttraumatic stress disorder. J. Clin. Psychiatry 65, 26-30 (2004).
106 Walker, E. A., Katon, W. J., Jemelka, R. P. & Roy-Bryne, P. P. Comorbidity of gastrointestinal complaints, depression, and anxiety in the Epidemiologic Catchment Area (ECA) Study. Am J Med 92, 26S-30S, doi:10.1016/0002-9343(92)90133-v (1992).
107 Valles-Colomer, M. et al. The neuroactive potential of the human gut microbiota in quality of life and depression. Nat Microbiol 4, 623-632, doi:10.1038/s41564-018-0337-x (2019).
108 Kelly, J. R. et al. Transferring the blues: Depression-associated gut microbiota induces neurobehavioural changes in the rat. J Psychiatr Res 82, 109-118, doi:10.1016/j.jpsychires.2016.07.019 (2016).
109 Jiang, H. et al. Altered fecal microbiota composition in patients with major depressive disorder. Brain Behav Immun 48, 186-194, doi:10.1016/j.bbi.2015.03.016 (2015).
110 Shen, Y. et al. Analysis of gut microbiota diversity and auxiliary diagnosis as a biomarker in patients with schizophrenia: A cross-sectional study. Schizophrenia research 197, 470-477, doi:10.1016/j.schres.2018.01.002 (2018).

1. A composition comprising one or more isolated, non-pathogenic queuine-producing bacterial strains or an isolated product derived therefrom, wherein the one or more isolated, non-pathogenic queuine-producing bacterial strains comprise live bacteria or dead bacteria, or wherein the isolated product derived therefrom comprises culture medium in which said one or more isolated, non-pathogenic bacterial strains have been cultured.

2. (canceled)

3. The composition of claim 1, wherein the isolated product derived therefrom comprises a purified polypeptide produced by the one or more bacterial strains.

4. The composition of claim 1, further comprising a pharmaceutically acceptable carrier, wherein the one or more isolated non-pathogenic queuine-producing bacterial strains or an isolated product derived therefrom is present in an amount effective to alter queuine levels in a subject in need thereof.

5. A pharmaceutical composition comprising queuine, an analog, derivative or precursor thereof, or a combination of any of these, in an amount effective to alter queuine levels in a subject in need thereof, and a pharmaceutically acceptable carrier.

6. The pharmaceutical composition of claim 5, wherein the queuine, analog, derivative or precursor is isolated from a queuine-producing bacterial strain or culture medium in which a queuine-producing bacterial strain has been cultured.

7. The composition of claim 1, wherein the at least one isolated non-pathogenic queuine producing bacteria is a human gut bacteria.

8. The composition of claim 1, wherein the at least one isolated non-pathogenic queuine-producing bacteria belongs to a species selected from Acetobacter pasteurianus, Achromobacter xylosoxidans, Acidaminococcus fermentans, Acidaminococcus intestini, Acinetobacter baumannii, Acinetobacter calcoaceticus, Acinetobacter junii, Acinetobacter lwoffii, Acinetobacter pittii, Acinetobacter radioresistens, Acinetobacter schindleri, Acinetobacter towneri, Acinetobacter ursingii, Acinetobacter variabilis, Adlercreutzia equolifaciens, Aeribacillus pallidus, Aeromonas caviae, Aeromonas enteropelogenes, Aeromonas hydrophila, Aeromonas jandaei, Aeromonas salmonicida, Aeromonas schubertii, Aeromonas veronii, Aggregatibacter aphrophilus, Akkermansia muciniphila, Alistipes onderdonkii, Alistipes putredinis, Allisonella histaminiformans, Anaeroglobus geminatus, Anaerostipes caccae, Anaerostipes hadrus, Aneurinibacillus aneurinilyticus, Aneurinibacillus migulanus, Anoxybacillus flavithermus, Asaccharobacter celatus, Bacillus altitudinis, Bacillus amyloliquefaciens, Bacillus aquimaris, Bacillus atrophaeus, Bacillus badius, Bacillus bataviensis, Bacillus cereus, Bacillus circulans, Bacillus clausii, Bacillus coagulans, Bacillus cohnii, Bacillus endophyticus, Bacillus firmus, Bacillus flexus, Bacillus fordii, Bacillus galactosidilyticus, Bacillus halodurans, Bacillus infantis, Bacillus koreensis, Bacillus kyonggiensis, Bacillus lentus, Bacillus licheniformis, Bacillus litoralis, Bacillus marisflavi, Bacillus megaterium, Bacillus mojavensis, Bacillus mycoides, Bacillus nealsonii, Bacillus okuhidensis, Bacillus pseudofirmus, Bacillus pseudomycoides, Bacillus pumilus, Bacillus simplex, Bacillus sonorensis, Bacillus subterraneus, Bacillus subtilis, Bacillus thuringiensis, Bacillus timonensis, Bacillus vallismortis, Bacillus vietnamensis, Bacillus weihenstephanensis, Bacteroides caccae, Bacteroides cellulosilyticus, Bacteroides clarus, Bacteroides coprocola, Bacteroides dorei, Bacteroides eggerthii, Bacteroides faecis, Bacteroides fragilis, Bacteroides intestinalis, Bacteroides massiliensis, Bacteroides nordii, Bacteroides ovatus, Bacteroides plebeius, Bacteroides salyersiae, Bacteroides stercoris, Bacteroides thetaiotaomicron, Bacteroides uniformis, Bacteroides vulgatus, Bacteroides xylanisolvens, Bacteroides xylanolyticus, Barnesiella intestinihominis, Barnesiella viscericola, Bilophila wadsworthia, Blautia luti, Bordetella bronchiseptica, Bordetella trematum, Brenneria alni, Brevibacillus agri, Brevibacillus brevis, Brevibacillus choshinensis, Brevibacillus formosus, Brevibacillus laterosporus, Brevibacillus parabrevis, Brevundimonas diminuta, Butyricimonas virosa, Campylobacter coli, Campylobacter concisus, Campylobacter curvus, Campylobacter gracilis, Campylobacter jejuni, Campylobacter showae, Campylobacter ureolyticus, Cedecea lapagei, Cedecea neteri, Chromohalobacter japonicus, Citrobacter amalonaticus, Citrobacter braakii, Citrobacter farmeri, Citrobacter freundii, Citrobacter gillenii, Citrobacter koseri, Citrobacter murliniae, Citrobacter youngae, Clostridium acetireducens, Clostridium bartlettii, Clostridium beijerinckii, Clostridium botulinum, Clostridium butyricum, Clostridium carboxidivorans, Clostridium colicanis, Clostridium diolis, Clostridium disporicum, Clostridium novyi, Clostridium ramosum, Clostridium sporogenes, Clostridium thermocellum, Coprococcus catus, Coprococcus eutactus, Cronobacter sakazakii, Delftia tsuruhatensis, Desulfovibrio desulfuricans, Desulfovibrio fairfieldensis, Desulfovibrio piger, Dialister invisus, Dialister pneumosintes, Enterobacter aerogenes, Enterobacter asburiae, Enterobacter cloacae, Enterobacter hormaechei, Enterobacter kobei, Enterobacter ludwigii, Enterorhabdus caecimuris, Erysipelatoclostridium ramosum, Escherichia coli, Escherichia fergusonii, Escherichia hermannii, Escherichia marmotae, Geobacillus stearothermophilus, Haemophilus influenzae, Haemophilus pittmaniae, Hafnia alvei, Halobacillus dabanensis, Halobacillus karajensis, Halobacillus salinus, Halobacillus trueperi, Helicobacter pylori, Intestinibacter bartlettii, Klebsiella oxytoca, Klebsiella pneumoniae, Klebsiella variicola, Kluyvera cryocrescens, Kluyvera georgiana, Kosakonia cowanii, Kushneria sinocarnis, Lachnospira pectinoschiza, Lachnotalea glycerini, Lactobacillus mali, Leclercia adecarboxylata, Lelliottia amnigena, Litorilituus sediminis, Lysinibacillus boronitolerans, Lysinibacillus fusiformis, Lysinibacillus massiliensis, Lysinibacillus sphaericus, Lysinibacillus xylanilyticus, Lysobacter soli, Megasphaera elsdenii, Megasphaera micronuciformis, Micrococcus lylae, Mitsuokella jalaludinii, Moellerella wisconsensis, Monoglobus pectinilyticus, Moraxella osloensis, Morganella morganii, Neisseria canis, Neisseria cinerea, Neisseria elongata, Neisseria flavescens, Neisseria gonorrhoeae, Neisseria macacae, Neisseria meningitidis, Neisseria mucosa, Neisseria perflava, Neisseria subflava, Nosocomiicoccus massiliensis, Noviherbaspirillum denitrificans, Oceanobacillus iheyensis, Oceanobacillus oncorhynchi, Oceanobacillus sojae, Ochrobactrum anthropi, Odoribacter splanchnicus, Oxalobacter formigenes, Paenibacillus alvei, Paenibacillus amylolyticus, Paenibacillus barcinonensis, Paenibacillus barengoltzii, Paenibacillus daejeonensis, Paenibacillus dendritiformis, Paenibacillus glucanolyticus, Paenibacillus illinoisensis, Paenibacillus lactis, Paenibacillus larvae, Paenibacillus lautus, Paenibacillus macerans, Paenibacillus naphthalenovorans, Paenibacillus odorifer, Paenibacillus pabuli, Paenibacillus pasadenensis, Paenibacillus polymyxa, Paenibacillus rhizosphaerae, Paenibacillus stellifer, Paenibacillus thiaminolyticus, Paenibacillus typhae, Pantoea agglomerans, Parabacteroides distasonis, Parabacteroides goldsteinii, Parabacteroides gordonii, Parabacteroides johnsonii, Parabacteroides merdae, Paraprevotella clara, Parasutterella excrementihominis, Peptoniphilus asaccharolyticus, Peptoniphilus indolicus, Planococcus rifietoensis, Porphyromonas asaccharolytica, Porphyromonas bennonis, Porphyromonas somerae, Prevotella bivia, Prevotella buccae, Prevotella buccalis, Prevotella copri, Prevotella timonensis, Proteus mirabilis, Proteus penneri, Proteus vulgaris, Providencia alcalifaciens, Providencia heimbachae, Providencia rettgeri, Providencia stuartii, Pseudomonas aeruginosa, Pseudomonas alcaligenes, Pseudomonas bauzanensis, Pseudomonas caricapapayae, Pseudomonas chlororaphis, Pseudomonas fluorescens, Pseudomonas fragi, Pseudomonas fulva, Pseudomonas gessardii, Pseudomonas japonica, Pseudomonas libanensis, Pseudomonas lundensis, Pseudomonas luteola, Pseudomonas migulae, Pseudomonas monteilii, Pseudomonas mosselii, Pseudomonas oleovorans, Pseudomonas oryzihabitans, Pseudomonas putida, Pseudomonas rhodesiae, Pseudomonas saudiphocaensis, Pseudomonas stutzeri, Pseudomonas taetrolens, Pseudomonas tolaasii, Pseudomonas xanthomarina, Psychrobacter phenylpyruvicus, Raoultella ornithinolytica, Raoultella planticola, Roseomonas gilardii, Roseomonas mucosa, Ruminococcus albus, Ruminococcus callidus, Ruminococcus flavefaciens, Ruminococcus lactaris, Ruminococcus torques, Salinisphaera halophila, Salinivibrio costicola, Salmonella enterica, Salmonella enteritidis, Salmonella typhi, Selenomonas ruminantium, Selenomonas sputigena, Senegalimassilia anaerobia, Serratia marcescens, Serratia ureilytica, Shewanella xiamenensis, Shigella boydii, Shigella dysenteriae, Shigella flexneri, Shigella sonnei, Sphingomonas aerolata, Staphylococcus arlettae, Staphylococcus aureus, Staphylococcus auricularis, Staphylococcus capitis, Staphylococcus caprae, Staphylococcus carnosus, Staphylococcus cohnii, Staphylococcus condimenti, Staphylococcus devriesei, Staphylococcus epidermidis, Staphylococcus equorum, Staphylococcus gallinarum, Staphylococcus haemolyticus, Staphylococcus hominis, Staphylococcus hyicus, Staphylococcus intermedius, Staphylococcus kloosii, Staphylococcus lentus, Staphylococcus lugdunensis, Staphylococcus nepalensis, Staphylococcus pasteuri, Staphylococcus petrasii, Staphylococcus pettenkoferi, Staphylococcus saccharolyticus, Staphylococcus saprophyticus, Staphylococcus schleiferi, Staphylococcus sciuri, Staphylococcus simiae, Staphylococcus simulans, Staphylococcus succinus, Staphylococcus vitulinus, Staphylococcus warneri, Staphylococcus xylosus, Stenotrophomonas acidaminiphila, Stenotrophomonas maltophilia, Stenotrophomonas rhizophila, Streptococcus australis, Streptococcus bovis, Streptococcus equinus, Streptococcus gallolyticus, Streptococcus infantarius, Streptococcus infantis, Streptococcus lutetiensis, Streptococcus mitis, Streptococcus mutans, Streptococcus oralis, Streptococcus peroris, Streptococcus pseudopneumoniae, Streptococcus salivarius, Streptococcus sobrinus, Streptococcus thermophilus, Streptococcus tigurinus, Streptococcus vestibularis, Succiniclasticum ruminis, Terribacillus aidingensis, Terribacillus halophilus, Thermotalea metallivorans, Turicibacter sanguinis, Veillonella atypica, Veillonella denticariosi, Veillonella dispar, Veillonella parvula, Vibrio cholerae, Victivallis vadensis, Virgibacillus massiliensis, Yersinia bercovieri, Yersinia enterocolitica, Yersinia intermedia, Yersinia kristensenii, Yersinia mollaretii, and combinations thereof.

9. The composition of claim 1, wherein the one or more non-pathogenic queuine producing bacteria is a human gut bacteria, and comprises a 16S rRNA sequence at least about 97% identical to a 16S rRNA sequence selected from SEQ ID NOs 1-406.

10. The composition of claim 1, wherein the at least one isolated non-pathogenic queuine producing bacteria is a human gut bacteria that encodes within its genome and expresses in the human gastrointestinal tract at least one queuine biosynthesis enzyme selected from folE (GTP cyclohydrolase), QueD (6-carboxy-5,6,7,8-tetrahydrobiopterin synthase), QueE (7-carboxy-7-deazaguanine synthase), QueC (7-cyano-7-deazaguanine synthase, PreQ0 synthase), QueF (7-cyano-7-deazaguanine reductase, PreQ0 reductase), tgt or btgt (tRNA guanine transglycosylase, bacterial tRNA guanine transglycosylase), QueA (S-adenosylmethionine:tRNA ribosyltransferase-isomerase), and QueG or QueH (epoxyqueuosine reductase).

11. The composition of claim 1, wherein the at least one isolated non-pathogenic queuine producing bacteria is a human gut bacteria that encodes within its genome and expresses in the human gastrointestinal tract at least one queuine biosynthesis enzyme, wherein the amino acid sequence encoded by the at least one queuine biosynthesis gene is at least 90% similar to a sequence selected from SEQ ID NOs 3660-82283.

12. The composition of claim 1, wherein the at least one isolated non-pathogenic queuine producing bacteria is a human gut bacteria belongs to species selected from Acidaminococcus fermentans, Adlercreutzia equolifaciens, Akkermansia muciniphila, Alloprevotella tannerae, Anaerostipes caccae, Anaerostipes hadrus, Arcobacter butzleri, Bacteroides caccae, Bacteroides cellulosilyticus, Bacteroides clarus, Bacteroides coprophilus, Bacteroides dorei, Bacteroides eggerthii, Bacteroides faecis, Bacteroides fragilis, Bacteroides massiliensis, Bacteroides nordii, Bacteroides oleiciplenus, Bacteroides ovatus, Bacteroides plebeius, Bacteroides salanitronis, Bacteroides salyersiae, Bacteroides stercoris, Bacteroides thetaiotaomicron, Bacteroides uniformis, Bacteroides vulgatus, Bacteroides xylanisolvens, Barnesiella intestinihominis, Bilophila wadsworthia, Butyrivibrio crossotus, Campylobacter curvus, Citrobacter freundii, Citrobacter koseri, Clostridium bartelettii, Clostridium ramosum, Coprobacter fastidiosus, Coprococcus catus, Coprococcus eutactus, Desulfovibrio piger, Dialister invisus, Dialister succinatiphilus, Enterobacter aerogenes, Enterobacter cancerogenus, Enterobacter cloacae, Enterorhabdus caecimuris, Escherichia coli, Eubacterium hallii, Fusobacterium mortiferum, Haemophilus pittmaniae, Haemophilus sputorum, Hafnia alvei, Klebsiella oxytoca, Klebsiella pneumoniae, Klebsiella variicola, Megamonas funiformis, Megamonas rupellensis, Megasphaera elsdenii, Megasphaera micronuciformis, Mitsuokella multacida, Odoribacter laneus, Odoribacter splanchnicus, Oxalobacter formigenes, Parabacteroides distasonis, Porphyromonas asaccharolytica, Porphyromonas uenonis, Ruminococcus callidus, Ruminococcus torques, Shigella sonnei, Streptococcus infantis, Streptococcus mitis, Streptococcus oralis, Streptococcus pneumoniae, Streptococcus tigurinus, Turicibacter sanguinis, Veillonella atypica, Veillonella dispar, Veillonella parvula, Dysgonomonas mossii, Proteus mirabilis, Veillonella ratti, and combinations thereof, and wherein the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.

13. The composition of claim 1, wherein the at least one isolated non-pathogenic queuine producing bacteria is a human gut bacteria with a 16S rRNA sequence at least about 97% identical to a 16S rRNA sequence selected from SEQ ID NOs 1-78, and the at least one isolated non-pathogenic queuine producing bacteria encodes within its genome and expresses in the human gastrointestinal tract at least one queuine biosynthesis enzyme with an amino acid sequence at least 90% identical to a sequence selected from SEQ ID NOs 3660-82283.

14. The composition of claim 5, wherein the queuine precursor is epoxyqueuine and/or cobalamin.

15. The composition of claim 5, wherein the queuine analogs are selected from queuosine, a mannosyl queuosine, galactosyl queuosine, glutamyl queuosine, mannosylqueuine, galactosylqueuine, and aminoacylated derivatives such as glutamylqueuine.

16. The composition of claim 1, wherein the composition is formulated in a capsule, a tablet, a caplet, a pill, a troche, a lozenge, a powder, a granule, a nutraceutical, a medical food, or a combination thereof.

17. The composition of claim 1, formulated for delivery to the gut.

18. The composition of claim 1, further comprising a prebiotic.

19. The composition of claim 1, further comprising a different composition in an amount effective to treat a CNS disease or disorder.

20. (canceled)

21. A method of increasing queuine levels in a subject in need thereof, the method comprising administering to the subject a composition of claim 1 in an amount effective to increase queuine levels in the subject.

22.-47. (canceled)

48. A composition comprising one or more isolated non-pathogenic endozepine-producing bacterial or yeast strains or an isolated product derived therefrom.

49.-108. (canceled)
